(19) United States
(12) Patent Application Publication
(10) Pub. No.: US 2024/0099993 A1
(43) Pub. Date: Mar. 28, 2024

(54) ARTIFICIALLY SYNTHESIZED SPHINGOSINE DERIVATIVE LIPOID MONOMER AND USE OF SAME FOR DELIVERING NUCLEIC ACID

(71) Applicant: INSTITUTE OF BASIC MEDICAL SCIENCES CHINESE ACADEMY OF MEDICAL SCIENCES, Beijing (CN)

(72) Inventors: Chengyu JIANG, Beijing (CN); Yuhao QIN, Beijing (CN); Xiaoyun LI, Beijing (CN); Cong ZHANG, Beijing (CN)

(21) Appl. No.: 18/506,138

(22) Filed: Nov. 10, 2023

Related U.S. Application Data
(63) Continuation of application No. 17/042,931, filed on Sep. 29, 2020.

(30) Foreign Application Priority Data
Mar. 29, 2018 (WO) PCT/CN2018/081155

(51) Int. Cl. A61K 31/133; A61K 9/00; A61K 31/14; A61K 31/661; A61K 31/7016; A61K 31/702; A61K 47/18; A61K 47/24; A61P 35/00; C07C 69/003; C07C 69/587; C07C 215/10; C07C 233/18; C07F 9/10; C12N 15/113

(52) U.S. Cl. CPC A61K 31/133; A61K 9/0053; A61K 31/14; A61K 31/661; A61K 31/7016; A61K 31/702; A61K 47/18; A61K 47/24; A61P 35/00; C07C 69/003; C07C 69/587; C07C 215/10; C07C 233/18; C07F 9/10; C12N 15/113; C12N 2310/141; C12N 2320/32

(57) ABSTRACT

The invention provides an artificially synthesized single sphingosine lipid and use of delivering a nucleic acid thereof. More particularly, the invention provides Use or method for delivering a nucleic acid to a cell or a subject using a compound of Formula (I), a stereoisomer or a pharmaceutical acceptable salt thereof, or a combination comprising a compound of Formula (I), a stereoisomer or a pharmaceutical acceptable salt thereof,

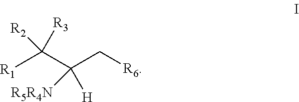

This application is a continuation application of U.S. application Ser. No. 17/042,931 filed Sep. 29, 2020, which is a continuation of PCT Patent Application No. PCT/CN2019/080519, filed on Mar. 29, 2019, which claims priority to PCT Application No. PCT/CN2018/081155, filed on Mar. 29, 2018, each of which is hereby incorporated by reference in its entirety.

### SEQUENCE LISTING

A copy of the Sequence Listing is submitted with the specification electronically via EFS-Web as an ASCII formatted sequence listing with a file name of “074844-8006US02-SL-20201208_ST25”, a creation date of Dec. 8, 2020, and a size of 1,601 bytes. The sequence listing contained in this ASCII formatted document is part of the specification and is herein incorporated by reference in its entirety.

### TECHNICAL FIELD

The present application generally relates to a delivery means of nucleic acid therapy, more particularly, relates to a more efficient delivery carrier and delivery means.

### BACKGROUND

Nucleic acid, as the main genetic material of living organisms, also has its unique potential for drug development. Currently, there are 6 types of nucleic acid drugs approved by the FDA, including Formavirsen (ISIS PHARMS INC, NDA: 20-961), mainly for the treatment of cytomegalovirus retinitis; Pagatanib (VALEANT PHARMS LLC, NDA: 21-756), mainly for neovascular age-related macular degeneration; Mipomethan (KASTLE THERAPS LLC, NDA: 203458), commonly for Homozygous familial hypercholesterolemia; Exondys 51 (SAREPTA THERAPS INC, NDA: 206488), for Duchenne muscular dystrophy; defibrotide-sodium (JAZZ PHAEMS INC, NDA: 208114), for the treatment of hepatic vein occlusive disease with renal or pulmonary dysfunction after hematopoiesis; Nusinersen (BIOGEN IDEC, NDA: 209531), for the treatment of spinal muscular atrophy; Patisiran (ALNYLAM PHARMS INC, NDA: 210922), for amyloidosis related to hereditary hyperthyroidism protein. These drugs are all administered by injection, and the drug delivery efficiency is relatively low.

From our previous research, there are hundreds of single lipids in the herbal decoctosome. In the further experiments of the present application, it was found that sphingosine lipids had better efficiency in the delivery process. Therefore, a series of sphingosine lipids and related derivatives had been artificially synthesized to specifically explore the delivery effect of sphingosine lipids. It was concluded that sphingosines and derivative lipids thereof could effectively deliver small RNAs.

### SUMMARY OF INVENTION

The present application is partly based on the inventors' discovery of a series of single sphingosine lipid. It was found that single sphingosine can be used as a carrier to efficiently deliver small RNA into cells.

The present application discovered that single sphingosine lipids can efficiently deliver small RNAs as drugs into cells without any toxicity to the body and significantly improve the delivery efficiency and medicinal value of nucleic acid drugs.

The sphingosine lipids and its single derivative can effectively deliver sRNA into cells. Functional small RNA with fluorescent FAM label (sRNA-PGY6) was used as a marker to detect the fluorescence shift after delivery to cells. Compared with free uptake, the experimental group of sphingosine lipid and its single derivative delivering small RNA had significant fluorescence shift, indicating that bencaosome formed by the sphingosine lipid and its single derivative can more effectively deliver small RNAs into cells to perform corresponding functions.

The present application provides the following contents:
1. Use or method for delivering a nucleic acid to a cell or a subject using a compound of Formula (I), a stereoisomer or a pharmaceutical acceptable salt thereof, or a combination comprising a compound of Formula (I), a stereoisomer or a pharmaceutical acceptable salt thereof,

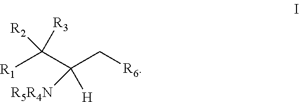

wherein:
R1 is selected from C₁₋₂₀alkyl or C₂₋₂₀alkenyl, which is optionally substituted by one to three hydroxyl groups;
R2 is hydrogen and R3 is hydroxyl; or
R2 and R3 together form oxo (═O);
R4 and R5 are independently selected from the group consisting of hydrogen, C₁₋₂₀alkyl, C₁₋₃₀alkylacyl and C₁₋₃₀alkenylacyl, said C₁₋₃₀alkylacyl and C₁₋₃₀alkenylacyl are optionally substituted by one to three groups selected from biotin acyl or hydroxyl;
or —NR4R5 group is a quaternary ammonium cation;
R6 is selected from the group consisting of hydrogen, hydroxyl, phosphate ester group, —O-glycosyl, Ganglioside, and aminoethoxyphosphonate ester group (NH₂—CH₂—CH₂—O—P(O)OH—).
2. The use or method of item 1, wherein:
R1 is selected from C₁₄₋₂₀alkyl or C₁₄₋₂₀alkenyl containing one double bond;
R4 and R5 are independently selected from the group consisting of hydrogen, C₁₋₆alkyl, C₆₋₃₀alkylacyl and C₆₋₃₀alkenylacyl, said C₆₋₃₀alkylacyl and the C₆₋₃₀alkenylacyl are optionally substituted at the terminal by one group selected from biotin acyl or hydroxyl, or optionally substituted at the α-carbon of the acyl by a hydroxyl.
3. The use or method of item 1 or 2, wherein R4 and R5 are independently selected from methyl.
4. The use or method of item 1 or 2, wherein at least one of R4 and R5 is hydrogen and the other is straight-chain C₆₋₃₀alkylacyl or straight-chain C₆₋₃₀alkenylacyl.
5. The use or method of any one of items 1-4, wherein at least one of R4 and R5 is hydrogen and the other is a straight-chain C₇₋₁₄alkyl; or both R4 and R5 are C₁₋₆alkyl.
6. The use or method of any one of items 1-5, wherein R1 is selected from a straight-chain C₁₄₋₂₀alkyl or a straight-chain C₁₄₋₂₀alkenyl containing one double bond.
7. The use or method of any one of items 1-6, wherein the glycosyl is glucosyl, lactosyl, or galactosyl.
8. The use or method of any one of items 1-7, wherein the —NR4R5 group is —NH₃⁺ or N(CH₃)₃⁺.
9. The use or method of any one of items 1-8, wherein the glycosyl is 1-β-D-glucosyl;
R2 is hydrogen, and R3 is hydroxyl;
R1 is selected from C₁₄₋₂₀alkenyl containing one double bond, and the alkenyl is immediately adjacent to the carbon atom to which R2 and R3 are attached.
10. The use or method of any one of the preceding items, wherein the compound has the following Formula (Ia):

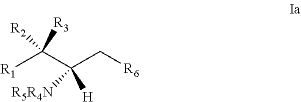

11. Use or method of delivering a nucleic acid to a cell or a subject using a compound selected from the following group, a stereoisomer or a pharmaceutically acceptable salt thereof, or a combination comprising the compound, a stereoisomer or a pharmaceutically acceptable salt thereof:

| No. | Structure |
|---|---|
| So-1 | 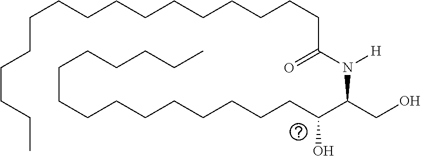 [SMILES: CCCCCCCCCCCCCCCCC(=O)N[C@@H](CO)[C@H](O)CCCCCCCCCCCCCCC] |
| So-2 | 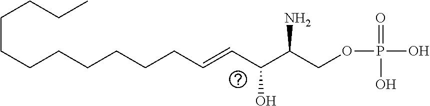 [SMILES: CCCCCCCCCCC/C=C/[C@@H](O)[C@@H](N)COP(=O)(O)O] |
| So-3 | 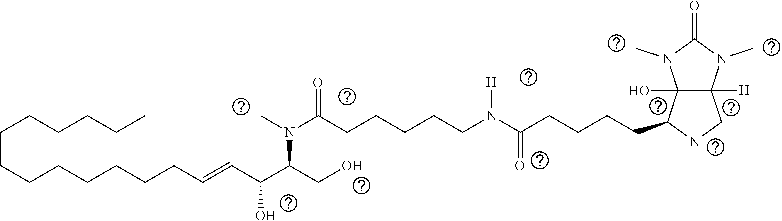 [SMILES: CCCCCCCCCCCCC/C=C/[C@@H](O)[C@H](CO)N(C)C(=O)CCCCCNC(=O)CCCC[C@@H]1NCC2N(C)C(=O)N(C)C21O] |
| So-4 | 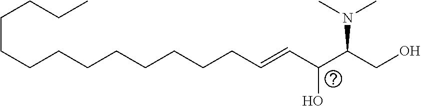 [SMILES: CCCCCCCCCCCCC/C=C/C(O)[C@H](CO)N(C)C] |
| So-5 | 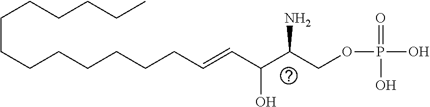 [SMILES: CCCCCCCCCCCCC/C=C/C(O)[C@@H](N)COP(=O)(O)O] |
| So-6 | 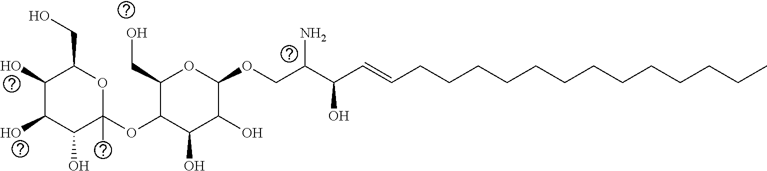 [SMILES: CCCCCCCCCCCCC/C=C/[C@@H](O)C(N)CO[C@@H]1O[C@H](CO)C(OC2(C)O[C@H](CO)[C@H](O)[C@H](O)[C@H]2O)[C@H](O)C1O] |
| So-7 | 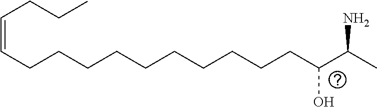 [SMILES: CCC/C=C\CCCCCCCCCC[C@@H](O)[C@H](C)N] |
| So-8 | 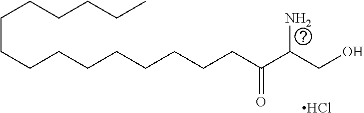 [SMILES: CCCCCCCCCCCCCCCC(=O)C(N)CO ; Cl] |
| So-9 | 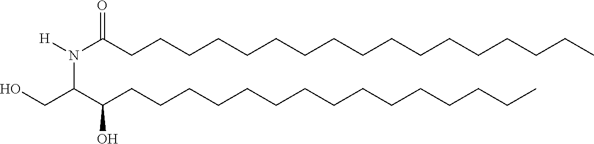 [SMILES: CCCCCCCCCCCCCCCCCC(=O)NC(CO)[C@H](O)CCCCCCCCCCCCCCC] |
| So-10 | 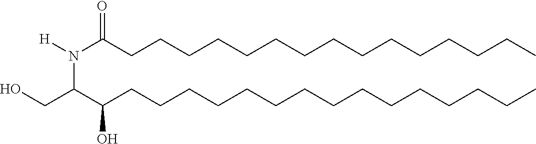 [SMILES: CCCCCCCCCCCCCCCC(=O)NC(CO)[C@H](O)CCCCCCCCCCCCCCC] |
| So-11 | 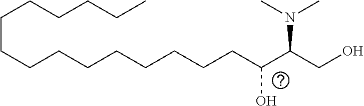 [SMILES: CCCCCCCCCCCCCCC[C@@H](O)[C@H](CO)N(C)C] |
| So-12 | 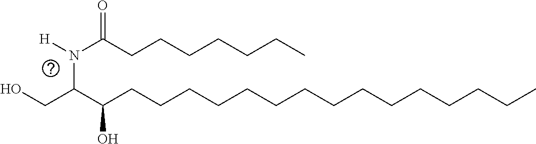 [SMILES: CCCCCCCCCCCCCCC[C@@H](O)C(CO)NC(=O)CCCCCCC] |
| So-13 | 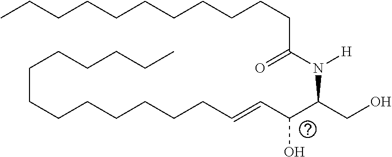 [SMILES: CCCCCCCCCCCCC/C=C/[C@@H](O)[C@H](CO)NC(=O)CCCCCCCCCCC] |
| So-14 | 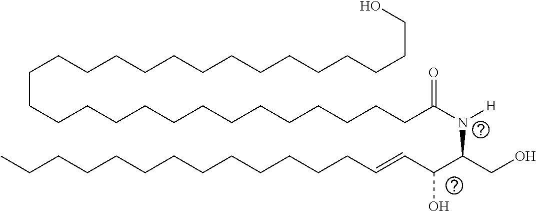 [SMILES: CCCCCCCCCCCCC/C=C/[C@@H](O)[C@H](CO)NC(=O)CCCCCCCCCCCCCCCCCCCCCCCCCCCCCO] |
| So-15 | 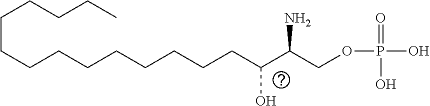 [SMILES: CCCCCCCCCCCCCC[C@@H](O)[C@@H](N)COP(=O)(O)O] |
| So-16 | 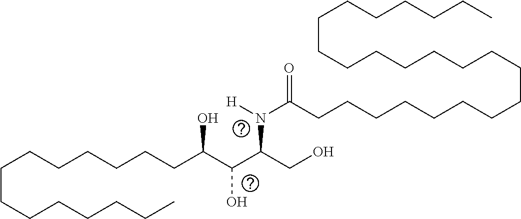 [SMILES: CCCCCCCCCCCCCCCCCCCCCCCC(=O)N[C@@H](CO)[C@H](O)[C@H](O)CCCCCCCCCCCCCC] |
| So-17 | 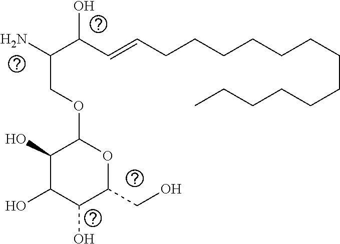 [SMILES: CCCCCCCCCCCCC/C=C/C(O)C(N)COC1O[C@H](CO)[C@H](O)C(O)[C@H]1O] |
| So-18 | 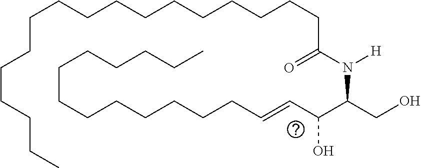 [SMILES: CCCCCCCCCCCCC/C=C/[C@@H](O)[C@H](CO)NC(=O)CCCCCCCCCCCCCCCCC] |
| So-19 | 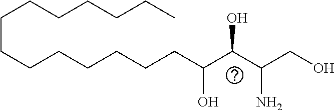 [SMILES: CCCCCCCCCCCCCCC(O)[C@@H](O)C(N)CO] |
| So-20 | 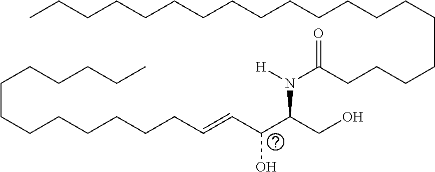 [SMILES: CCCCCCCCCCCCC/C=C/[C@@H](O)[C@H](CO)NC(=O)CCCCCCCCCCCCCCCCCCC] |
| So-21 | 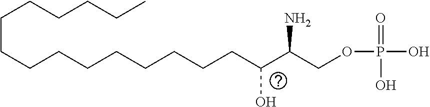 [SMILES: CCCCCCCCCCCCCCC[C@@H](O)[C@@H](N)COP(=O)(O)O] |
| So-23 | 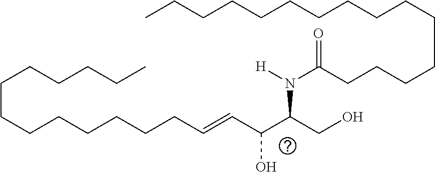 [SMILES: CCCCCCCCCCCCC/C=C/[C@@H](O)[C@H](CO)NC(=O)CCCCCCCCCCCCCCC] |
| So-25 | 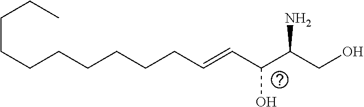 [SMILES: CCCCCCCCCC/C=C/[C@@H](O)[C@@H](N)CO] |
| So-26 | 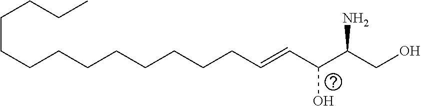 [SMILES: CCCCCCCCCCCCC/C=C/[C@@H](O)[C@@H](N)CO] |
| So-27 | 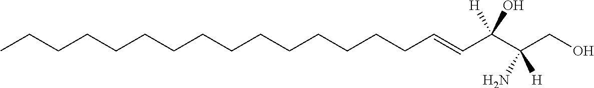 [SMILES: CCCCCCCCCCCCCCC/C=C/[C@@H](O)[C@@H](N)CO] |
| So-29 | 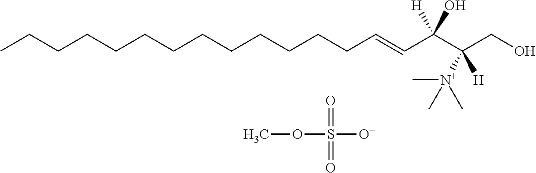 |
| So-30 | 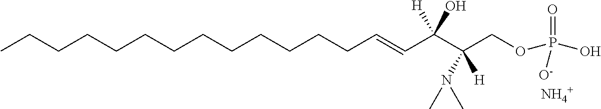 [SMILES: CCCCCCCCCCCCC/C=C/[C@@H](O)[C@H](COP(=O)([O-])O)N(C)C ; [NH4+]] |
| So-31 | 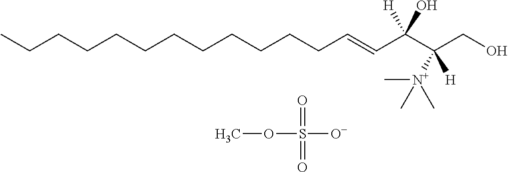 |
| So-32 | 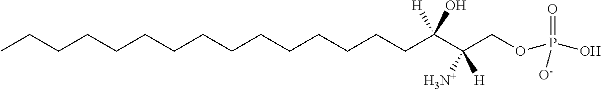 [SMILES: CCCCCCCCCCCCCCC[C@@H](O)[C@@H]([NH3+])COP(=O)([O-])O] |
| So-33 | 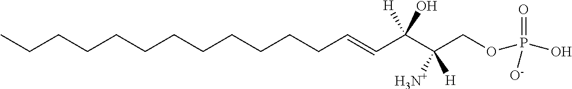 [SMILES: CCCCCCCCCCCC/C=C/[C@@H](O)[C@@H]([NH3+])COP(=O)([O-])O] |
| So-34 | 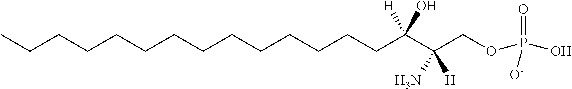 [SMILES: CCCCCCCCCCCCCC[C@@H](O)[C@@H]([NH3+])COP(=O)([O-])O] |
| So-35 | 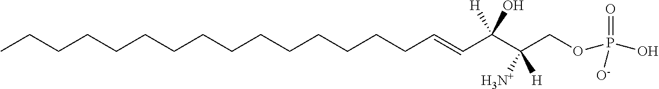 [SMILES: CCCCCCCCCCCCCCC/C=C/[C@@H](O)[C@@H]([NH3+])COP(=O)([O-])O] |
| So-36 | 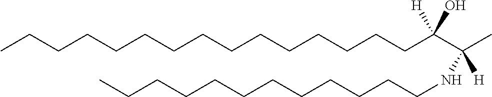 [SMILES: CCCCCCCCCCCCCCC[C@@H](O)[C@H](C)NCCCCCCCCCCCC] |
| So-37 | 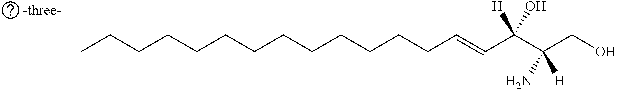 [SMILES: CCCCCCCCCCCCC/C=C/[C@H](O)[C@@H](N)CO] |
| So-38 | 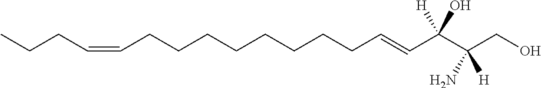 [SMILES: CCC/C=C\CCCCCCCC/C=C/[C@@H](O)[C@@H](N)CO] |
| So-40 | 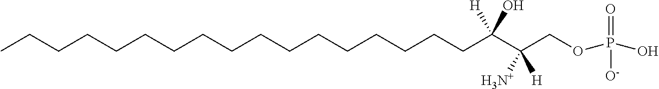 [SMILES: CCCCCCCCCCCCCCCCC[C@@H](O)[C@@H]([NH3+])COP(=O)([O-])O] |
| So-41 | 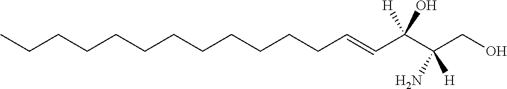 [SMILES: CCCCCCCCCCCC/C=C/[C@@H](O)[C@@H](N)CO] |
| So-42 | 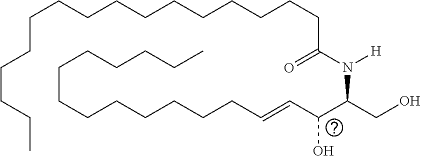 [SMILES: CCCCCCCCCCCCC/C=C/[C@@H](O)[C@H](CO)NC(=O)CCCCCCCCCCCCCCCC] |
| So-43 | 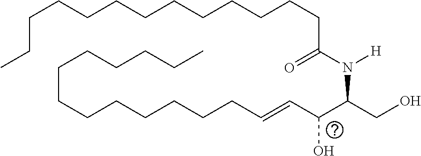 [SMILES: CCCCCCCCCCCCC/C=C/[C@@H](O)[C@H](CO)NC(=O)CCCCCCCCCCCCC] |
| So-44 | 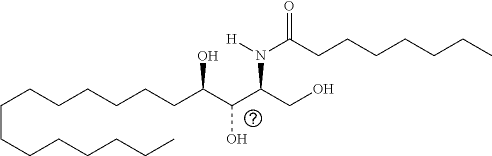 [SMILES: CCCCCCCCCCCCCC[C@@H](O)[C@@H](O)[C@H](CO)NC(=O)CCCCCCC] |
| So-45 | 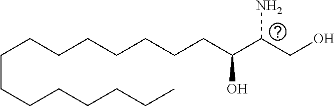 [SMILES: CCCCCCCCCCCCCCC[C@H](O)[C@H](N)CO] |
| So-46 | 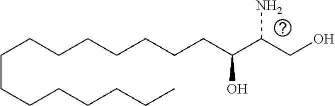 [SMILES: CCCCCCCCCCCCCCC[C@H](O)[C@H](N)CO] |
| So-47 | 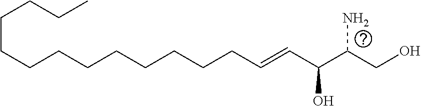 [SMILES: CCCCCCCCCCCCC/C=C/[C@H](O)[C@H](N)CO] |
| So-48 | 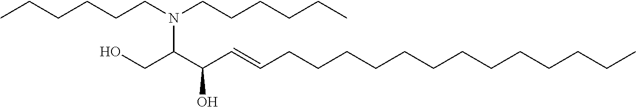 [SMILES: CCCCCCCCCCCCC/C=C/[C@@H](O)C(CO)N(CCCCCC)CCCCCC] |
| So-49 | 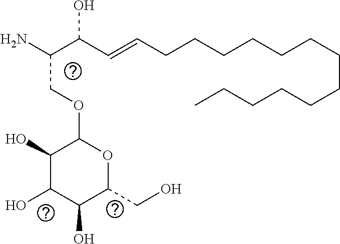 [SMILES: CCCCCCCCCCCCC/C=C/[C@@H](O)[C@@H](N)COC1O[C@H](CO)[C@@H](O)C(O)[C@H]1O] |
| So-50 | 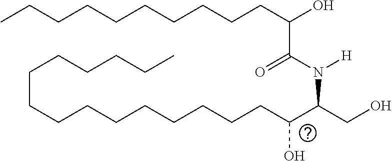 [SMILES: CCCCCCCCCCCCCCC[C@@H](O)[C@H](CO)NC(=O)C(O)CCCCCCCCCC] |
| So-51 | 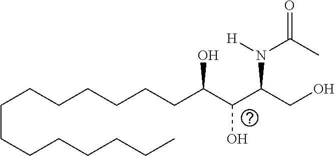 [SMILES: CCCCCCCCCCCCCC[C@@H](O)[C@@H](O)[C@H](CO)NC(C)=O] |
| So-52 | 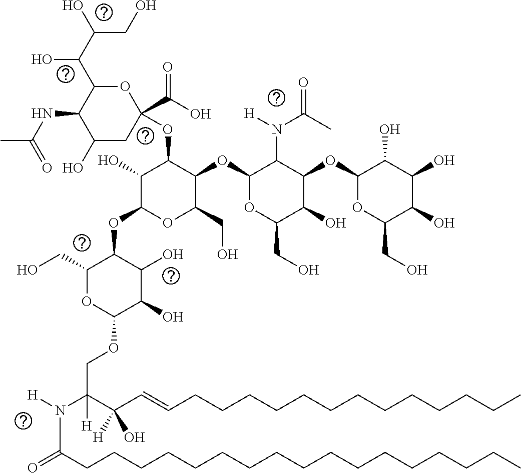 [SMILES: CCCCCCCCCCCCC/C=C/[C@@H](O)C(CO[C@@H]1O[C@H](CO)[C@@H](O[C@@H]2O[C@H](CO)[C@H](O[C@@H]3O[C@H](CO)[C@H](O)[C@H](O[C@@H]4O[C@H](CO)[C@H](O)[C@H](O)[C@H]4O)C3NC(C)=O)[C@H](O[C@]3(C(=O)O)CC(O)[C@@H](NC(C)=O)C(C(O)C(O)CO)O3)[C@H]2O)C(O)[C@H]1O)NC(=O)CCCCCCCCCCCCCCCCC] |
| So-53 | 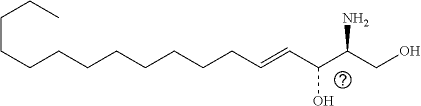 [SMILES: CCCCCCCCCCCC/C=C/[C@@H](O)[C@@H](N)CO] |
| So-54 | 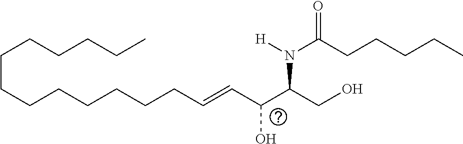 [SMILES: CCCCCCCCCCCCC/C=C/[C@@H](O)[C@H](CO)NC(=O)CCCCC] |
| So-55 | 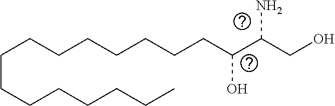 [SMILES: CCCCCCCCCCCCCCC[C@@H](O)[C@H](N)CO] |
| So-56 | 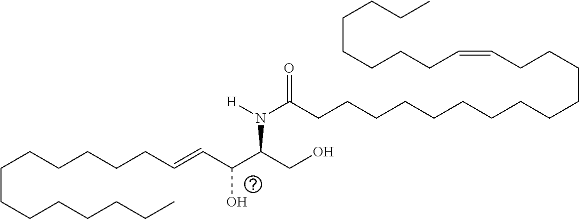 [SMILES: CCCCCCCC/C=C\CCCCCCCCCCCCCC(=O)N[C@@H](CO)[C@H](O)/C=C/CCCCCCCCCCCCC] |
| So-57 | 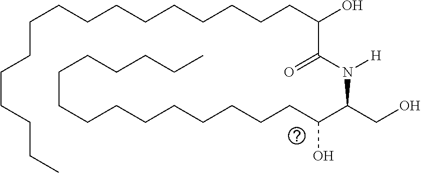 [SMILES: CCCCCCCCCCCCCCCCC(O)C(=O)N[C@@H](CO)[C@H](O)CCCCCCCCCCCCCCC] |
| So-58 | 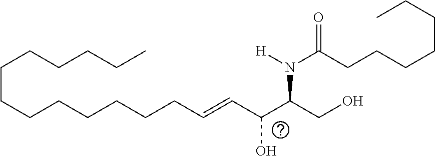 [SMILES: CCCCCCCCCCCCC/C=C/[C@@H](O)[C@H](CO)NC(=O)CCCCCCC] |
| So-59 | 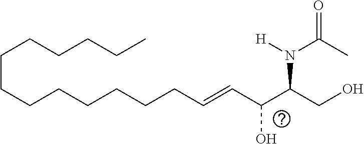 [SMILES: CCCCCCCCCCCCC/C=C/[C@@H](O)[C@H](CO)NC(C)=O] |
| So-60 | 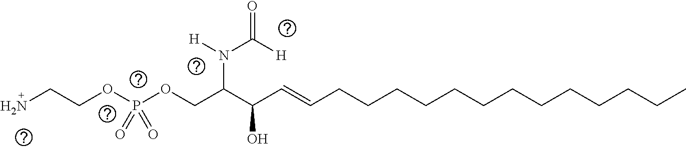 |
| So-61 | 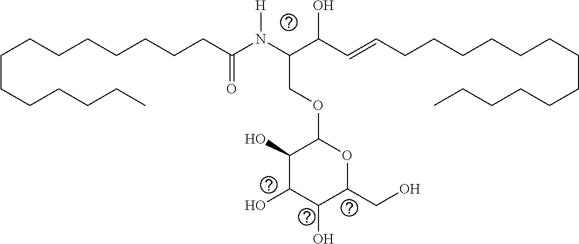 [SMILES: CCCCCCCCCCCCC/C=C/C(O)C(COC1OC(CO)C(O)C(O)[C@H]1O)NC(=O)CCCCCCCCCCCCCC] |
| So-62 | 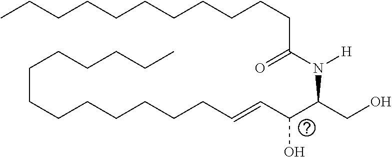 [SMILES: CCCCCCCCCCCCC/C=C/[C@@H](O)[C@H](CO)NC(=O)CCCCCCCCCCC] |
| So-63 | 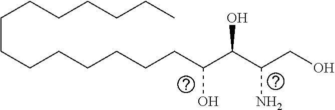 [SMILES: CCCCCCCCCCCCCC[C@@H](O)[C@@H](O)[C@@H](N)CO] |
| So-64 | 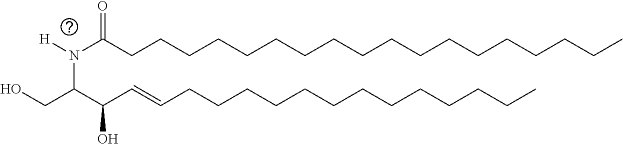 [SMILES: CCCCCCCCCCCCC/C=C/[C@@H](O)C(CO)NC(=O)CCCCCCCCCCCCCCCCCC] |
| So-65 | 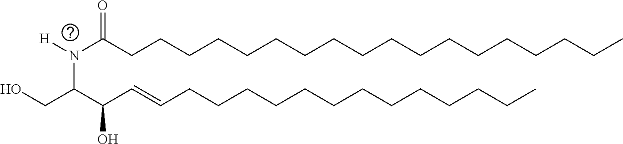 [SMILES: CCCCCCCCCCCCC/C=C/[C@@H](O)C(CO)NC(=O)CCCCCCCCCCCCCCCCCC] |
| So-66 | 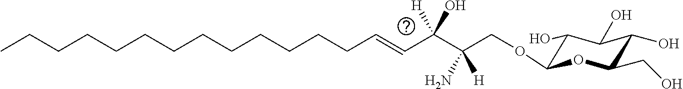 |
| So-67 | 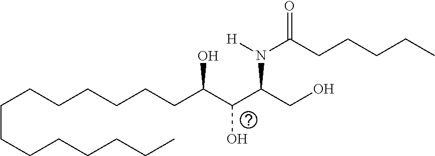 [SMILES: CCCCCCCCCCCCCC[C@@H](O)[C@@H](O)[C@H](CO)NC(=O)CCCCC] |
| So-68 | 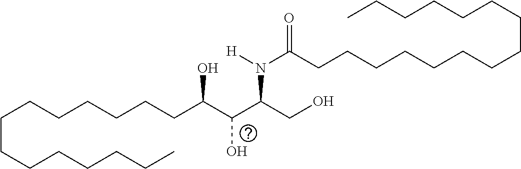 [SMILES: CCCCCCCCCCCCCCCC(=O)N[C@@H](CO)[C@H](O)[C@H](O)CCCCCCCCCCCCCC] |
| So-69 | 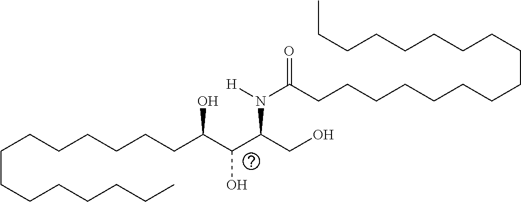 [SMILES: CCCCCCCCCCCCCCCCCC(=O)N[C@@H](CO)[C@H](O)[C@H](O)CCCCCCCCCCCCCC] |
| So-70 | 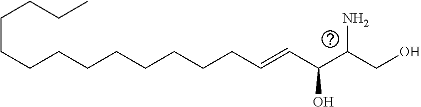 [SMILES: CCCCCCCCCCCCC/C=C/[C@H](O)C(N)CO] |
| So-71 | 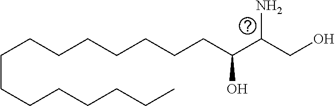 [SMILES: CCCCCCCCCCCCCCC[C@H](O)C(N)CO] |
| So-72 | 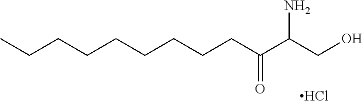 |
| So-73 | 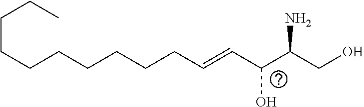 [SMILES: CCCCCCCCCC/C=C/[C@@H](O)[C@@H](N)CO] |
| So-74 | 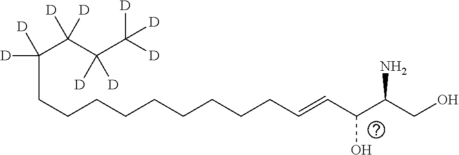 [SMILES: [2H]C([2H])([2H])C([2H])([2H])C([2H])([2H])C([2H])([2H])CCCCCCCCC/C=C/[C@@H](O)[C@@H](N)CO] |
| So-75 | 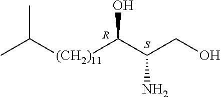 |
| No. 41 | 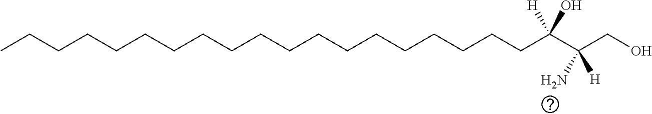 [SMILES: CCCCCCCCCCCCCCCCCCC[C@@H](O)[C@@H](N)CO] |
| No. 38 | 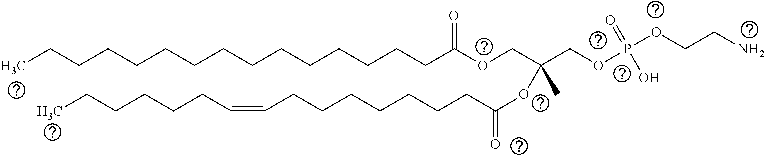 [SMILES: CCCCCC/C=C\CCCCCCCC(=O)O[C@](C)(COC(=O)CCCCCCCCCCCCCCC)COP(=O)(O)OCCN] |
| No. 48 | 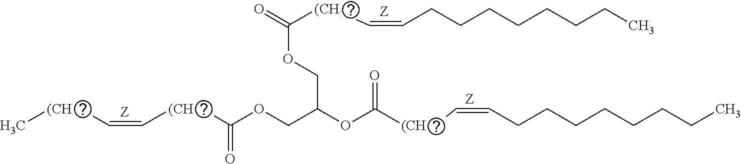 |

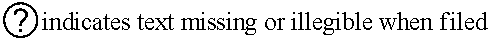

12. The use or method of any one of the preceding items, wherein the combination is a combination comprising any one or more of No.41, No.38, No.48, a combination comprising any one or more of No.41, No.38, No.48 and any one or more of the compound selecting from item 11, a combination comprising the following: No. 41+No. 38+sphingosine derivative So-1; No. 41+No. 38+sphingosine derivative So-2; No. 41+No. 38+sphingosine derivative So-3; No. 41+No. 38+sphingosine derivative So-4; No. 41+No. 38+sphingosine derivative So-5; No. 41+No. 38+sphingosine derivative So-6; No. 41+No. 38+sphingosine derivative So-7; No. 41+No. 38+sphingosine derivative So-8; No. 41+No. 38+sphingosine derivative So-9; No. 41+No. 38+sphingosine derivative So-10; No. 41+No. 38+sphingosine derivative So-11; No. 41+No. 38+sphingosine derivative So-12; No. 41+No. 38+sphingosine derivative So-13; No. 41+No. 38+sphingosine derivative So-14; No. 41+No. 38+sphingosine derivative So-15; No. 41+No. 38+sphingosine derivative So-45; No. 41+No. 38+sphingosine derivative So-46; No. 41+No. 38+sphingosine derivative So-47; No. 41+No. 38+sphingosine derivative So-48; No. 41+No. 38+sphingosine derivative So-49; No. 41+No. 38+sphingosine derivative So-50; No. 41+No. 38+sphingosine derivative So-51; No. 41+No. 38+sphingosine derivative So-52; No. 41+No. 38+sphingosine derivative So-53; No. 41+No. 38+sphingosine derivative So-54; No. 41+No. 38+sphingosine derivative So-55; No. 41+No. 38+sphingosine derivative So-56; No. 41+No. 38+sphingosine derivative So-57; No. 41+No. 38+sphingosine derivative So-58; No. 41+No. 38+sphingosine derivative So-59; No. 41+No. 38+sphingosine derivative So-60; No. 41+No. 38+sphingosine derivative So-61; No. 41+No. 38+sphingosine derivative So-62; No. 41+No. 38+sphingosine derivative So-63; No. 41+No. 38+sphingosine derivative So-64; No. 41+No. 38+sphingosine derivative So-65; No. 41+No. 38+sphingosine derivative So-66; No. 41+No. 38+sphingosine derivative So-67; No. 41+No. 38+sphingosine derivative So-68; No. 41+No. 38+sphingosine derivative So-69; No. 41+No. 38+sphingosine derivative So-70; No. 41+No. 38+sphingosine derivative So-71; No. 41+No. 38+sphingosine derivative So-72; No. 41+No. 38+sphingosine derivative So-73; No. 41+No. 38+sphingosine derivative So-74; No. 41+No. 38+sphingosine derivative So-75; No. 41+No. 38+No. 48+sphingosine derivative So-42; No. 41+No. 38+No. 48+sphingosine derivative So-43; No. 41+No. 38+No. 48+sphingosine derivative So-44; No. 41+No. 38+No. 48+sphingosine derivative So-45; No. 41+No. 38+No. 48+sphingosine derivative So-46; No. 41+No. 38+No. 48+sphingosine derivative So-47; No. 41+No. 38+No. 48+sphingosine derivative So-52; No. 41+No. 38+No. 48+sphingosine derivative So-56; No. 41+No. 38+No. 48+sphingosine derivative So-57; No. 41+No. 38+No. 48+sphingosine derivative So-58; No. 41+No. 38+No. 48+sphingosine derivative So-59; No. 41+No. 38+No. 48+sphingosine derivative So-60; No. 41+No. 38+No. 48+sphingosine derivative So-61; No. 41+No. 38+No. 48+sphingosine derivative So-62; No. 41+No. 38+No. 48+sphingosine derivative So-63; No. 41+No. 38+No. 48+sphingosine derivative So-64; No. 41+No. 38+No. 48+sphingosine derivative So-67; No. 41+No. 38+No. 48+sphingosine derivative So-68; No. 41+No. 38+No. 48+sphingosine derivative So-69; No. 41+No. 38+No. 48+sphingosine derivative So-70; No. 41+No. 38+any one or more of sphingosine derivative So-23, 25, 26, 27, 29, 30, 31, 32, 33, 34, 35, 36, 37, 38, 40, 41, 42, 43, 44, 70, 71 or 73; preferably, the compounds above are used at concentrations as shown in Table 1; preferably, the ratio of the compounds above is 0.1-10:0.1-10, 0.2-9:0.2-9, 0.3-8:0.3-8, 0.4-7:0.4-7, 0.5-6:0.5-6, 0.6-5:0.6-5, 0.7-4:0.7-4, 0.8-3:0.8-3, 0.9-2:0.9-2; more preferably 1:1.
13. The use or method of any one of the preceding items, wherein the nucleic acid is synthetic or purified, therapeutic or non-therapeutic, and/or diagnostic or non-diagnostic, for example selected from RNA or DNA, for example selected from single-stranded or double-stranded or partially double-stranded RNA or DNA;
preferably, when the nucleic acid is therapeutic or diagnostic, the nucleic acid is used to treat or diagnose a disease selected from the group consisting of: inflammatory diseases, pneumonia, myocarditis, acute and chronic gastritis, acute and chronic enteritis, acute and chronic hepatitis, acute and chronic nephritis, dermatitis, encephalitis, lymphitis, conjunctivitis, keratitis, iridocyclitis, otitis media, allergic rhinitis, asthma, pulmonary fibrosis, chronic obstructive pulmonary disease, allergic dermatitis, sickle cell disease, multiple sclerosis, systemic lupus erythematosus, lupus nephritis, lung cancer, gastric cancer, colorectal cancer, liver cancer, pancreatic cancer, cervical cancer, breast cancer, leukemia, multiple myeloma, diabetes, and gout.
14. The use or method of item 13, wherein the RNA is selected from the group consisting of messenger RNA (mRNA), rRNA (ribosomal RNA), tRNA (transfer RNA), heterogeneous nuclear RNA (hnRNA), small nuclear RNA (snRNA), small nucleolar RNA (snoRNA), small cytoplasmic RNA, small RNA, transfer-messenger RNA (tmRNA), telomerase RNA and antisense RNA, preferably small RNA.
15. The use or method of item 14, wherein the length of the small RNA is 14-32 bp, 16-28 bp, or 18-24 bp.
16. The use or method of item 15, wherein the small RNA comprises the sequence of GTTCAGAGTTCTACAGTCCGA (SEQ ID NO: 1).
17. The use or method of any one of the preceding items, wherein the delivery comprises treating the compound, a stereoisomer or a pharmaceutically acceptable salt or a combination comprising the compound of Formula (I), a stereoisomer or a pharmaceutically acceptable salt thereof or combination thereof, by heating method, reverse evaporation method, direct mixing, repeated freeze-thaw and/or thin film dispersion.
18. The use or method of item 17, wherein the heating method is conducted at a temperature of about 0° C. to about 100° C., about 25° C. to about 100° C., preferably about 80° C. to about 100° C., for example 4° C., 37° C., 60° C., 80° C. or 100° C.; the reverse evaporation method is conducted at a temperature of about 25° C. to about 70° C., preferably about 55° C. for a heating time of about 0 minutes to about 24 hours, about 5 minutes to about 20 hours, about 5 minutes to about 16 hours, about 5 minutes to about 10 hours, about 5 minutes to about 4 hours, or about 10 hours to about 1 hour, preferably 15 minutes.
19. The use of item 17, wherein the delivery comprises in vitro cell delivery or in vivo subject delivery.
20. The use or method of item 19, wherein the in vivo subject delivery comprises oral administration, intravenous administration such as injection or infusion, subcutaneous administration, intramuscular administration, intraarterial administration, intraperitoneal administration, intrapulmonary administration, intracerebral and intraspinal administration, intra-articular administration, intrasynovial administration, intrathecal administration, intra-trauma administration, and/or administration via inhalation paths such as intranasal, typically intravenous or subcutaneous administration.
21. Use of a compound of Formula (I), a stereoisomer or a pharmaceutically acceptable salt thereof, or a combination comprising a compound of Formula (I), a stereoisomer or a pharmaceutically acceptable salt thereof for the manufacture of a reagent for nucleic acid delivery,

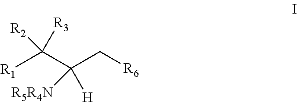

wherein:
R1 is selected from C₁₋₂₀alkyl or C₂₋₂₀alkenyl, which is optionally substituted by one to three hydroxyl groups;
R2 is hydrogen and R3 is hydroxyl; or
R2 and R3 together form oxo (═O);
R4 and R5 are independently selected from the group consisting of hydrogen, C₁₋₂₀alkyl, C₁₋₃₀alkylacyl and C₁₋₃₀alkenylacyl, said C₁₋₃₀alkylacyl and C₁₋₃₀alkenylacyl are optionally substituted by one to three groups selected from biotin acyl or hydroxyl;
or —NR4R5 group is a quaternary ammonium cation;
R6 is selected from the group consisting of hydrogen, hydroxyl, phosphate ester group, —O-glycosyl, Ganglioside, and aminoethoxyphosphonate ester group (NH₂—CH₂—CH₂—O—P(O)OH—).
22. The use of item 21, wherein:
R1 is selected from C₁₄₋₂₀alkyl or C₁₄₋₂₀alkenyl containing one double bond;
R4 and R5 are independently selected from the group consisting of hydrogen, C₆₋₃₀alkyl, C₆₋₃₀alkylacyl and C₆₋₃₀alkenylacyl, said C₆₋₃₀alkylacyl and the C₆₋₃₀alkenylacyl are optionally substituted at the terminal by one group selected from biotin acyl or hydroxyl, or optionally substituted at the α-carbon of the acyl by one hydroxyl.
23. The use of item 21 or 22, wherein R4 and R5 are independently selected from methyl.
24. The use of item 21 or 22, wherein at least one of R4 and R5 is hydrogen and the other is straight-chain C₆₋₃₀alkylacyl or straight-chain C₆₋₃₀alkenylacyl.
25. The use of any one of items 21-24, wherein at least one of R4 and R5 is hydrogen and the other is a straight-chain C₇₋₁₄alkyl; or both R4 and R5 are C₁₋₆alkyl.
26. The use of any one of items 21-25, wherein R1 is selected from a straight-chain C₁₄₋₂₀alkyl or a straight-chain C₁₄₋₂₀alkenyl containing one double bond.
27. The use of any one of items 21-26, wherein the glycosyl is glucosyl, lactosyl, or galactosyl.
28. The use of any one of items 21-27, wherein the glycosyl is 1-β-D-glucosyl;
R2 is hydrogen, and R3 is hydroxyl;
R1 is selected from C₁₄₋₂₀alkenyl containing one double bond, and the alkenyl is immediately adjacent to the carbon atom to which R2 and R3 are attached.
29. The use of any one of items 21-28, wherein the compound has the following Formula (Ia):

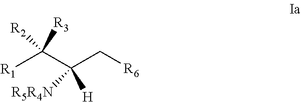

30. The use of any one of items 21-29, wherein the compound of Formula (I) is selected from one or more compounds of item 11.
31. The use of any one of items 21-30, wherein the combination is a combination comprising any one or more of No. 41, No. 38, No. 48, a combination comprising any one or more of No. 41, No. 38, No. 48 and any one or more of the compound selecting from item 7, a combination comprising the following: No. 41+No. 38+sphingosine derivative So-1; No. 41+No. 38+sphingosine derivative So-2; No. 41+No. 38+sphingosine derivative So-3; No. 41+No. 38+sphingosine derivative So-4; No. 41+No. 38+sphingosine derivative So-5; No. 41+No. 38+sphingosine derivative So-6; No. 41+No. 38+sphingosine derivative So-7; No. 41+No. 38+sphingosine derivative So-8; No. 41+No. 38+sphingosine derivative So-9; No. 41+No. 38+sphingosine derivative So-10; No. 41+No. 38+sphingosine derivative So-11; No. 41+No. 38+sphingosine derivative So-12; No. 41+No. 38+sphingosine derivative So-13; No. 41+No. 38+sphingosine derivative So-14; No. 41+No. 38+sphingosine derivative So-15; No. 41+No. 38+sphingosine derivative So-45; No. 41+No. 38+sphingosine derivative So-46; No. 41+No. 38+sphingosine derivative So-47; No. 41+No. 38+sphingosine derivative So-48; No. 41+No. 38+sphingosine derivative So-49; No. 41+No. 38+sphingosine derivative So-50; No. 41+No. 38+sphingosine derivative So-51; No. 41+No. 38+sphingosine derivative So-52; No. 41+No. 38+sphingosine derivative So-53; No. 41+No. 38+sphingosine derivative So-54; No. 41+No. 38+sphingosine derivative So-55; No. 41+No. 38+sphingosine derivative So-56; No. 41+No. 38+sphingosine derivative So-57; No. 41+No. 38+sphingosine derivative So-58; No. 41+No. 38+sphingosine derivative So-59; No. 41+No. 38+sphingosine derivative So-60; No. 41+No. 38+sphingosine derivative So-61; No. 41+No. 38+sphingosine derivative So-62; No. 41+No. 38+sphingosine derivative So-63; No. 41+No. 38+sphingosine derivative So-64; No. 41+No. 38+sphingosine derivative So-65; No. 41+No. 38+sphingosine derivative So-66; No. 41+No. 38+sphingosine derivative So-67; No. 41+No. 38+sphingosine derivative So-68; No. 41+No. 38+sphingosine derivative So-69; No. 41+No. 38+sphingosine derivative So-70; No. 41+No. 38+sphingosine derivative So-71; No. 41+No. 38+sphingosine derivative So-72; No. 41+No. 38+sphingosine derivative So-73; No. 41+No. 38+sphingosine derivative So-74; No. 41+No. 38+sphingosine derivative So-75; No. 41+No. 38+No. 48+sphingosine derivative So-42; No. 41+No. 38+No. 48+sphingosine derivative So-43; No. 41+No. 38+No. 48+sphingosine derivative So-44; No. 41+No. 38+No. 48+sphingosine derivative So-45; No. 41+No. 38+No. 48+sphingosine derivative So-46; No. 41+No. 38+No. 48+sphingosine derivative So-47; No. 41+No. 38+No. 48+sphingosine derivative So-52; No. 41+No. 38+No. 48+sphingosine derivative So-56; No. 41+No. 38+No. 48+sphingosine derivative So-57; No. 41+No. 38+No. 48+sphingosine derivative So-58; No. 41+No. 38+No. 48+sphingosine derivative So-59; No. 41+No. 38+No. 48+sphingosine derivative So-60; No. 41+No. 38+No. 48+sphingosine derivative So-61; No. 41+No. 38+No. 48+sphingosine derivative So-62; No. 41+No. 38+No. 48+sphingosine derivative So-63; No. 41+No. 38+No. 48+sphingosine derivative So-64; No. 41+No. 38+No. 48+sphingosine derivative So-67; No. 41+No. 38+No. 48+sphingosine derivative So-68; No. 41+No. 38+No. 48+sphingosine derivative So-69; No. 41+No. 38+No. 48+sphingosine derivative So-70; No. 41+No. 38+any one or more of sphingosine derivative So-23, 25, 26, 27, 29, 30, 31, 32, 33, 34, 35, 36, 37, 38, 40, 41, 42, 43, 44, 70, 71 or 73; preferably, the compounds above are used at concentrations as shown in Table 1; preferably, the ratio of the compounds above is 0.1-10:0.1-10, 0.2-9:0.2-9, 0.3-8:0.3-8; 0.4-7:0.4-7, 0.5-6:0.5-6, 0.6-5:0.6-5, 0.7-4:0.7-4, 0.8-3:0.8-3, 0.9-2:0.9-2; more preferably 1:1.
32. The use of any one of items 21-31, wherein the nucleic acid is synthetic or purified, therapeutic or non-therapeutic, and/or diagnostic or non-diagnostic, for example selected from RNA or DNA, for example selected from single-stranded or double-stranded or partially double-stranded RNA or DNA;
preferably, when the nucleic acid is therapeutic or diagnostic, the nucleic acid is used to treat or diagnose a disease selected from the group consisting of: inflammatory diseases, pneumonia, myocarditis, acute and chronic gastritis, acute and chronic enteritis, acute and chronic hepatitis, acute and chronic nephritis, dermatitis, encephalitis, lymphitis, conjunctivitis, keratitis, iridocyclitis, otitis media, allergic rhinitis, asthma, pulmonary fibrosis, chronic obstructive pulmonary disease, allergic dermatitis, sickle cell disease, multiple sclerosis, systemic lupus erythematosus, lupus nephritis, lung cancer, gastric cancer, colorectal cancer, liver cancer, pancreatic cancer, cervical cancer, breast cancer, leukemia, multiple myeloma, diabetes, and gout.
33. The use of item 32, wherein wherein the RNA is selected from the group consisting of messenger RNA (mRNA), rRNA (ribosomal RNA), tRNA (transfer RNA), heterogeneous nuclear RNA (hnRNA), small nuclear RNA (snRNA), small nucleolar RNA (snoRNA), small cytoplasmic RNA, small RNA, transfer-messenger RNA (tmRNA), telomerase RNA and antisense RNA, preferably small RNA.
34. The use of item 33, wherein the length of the small RNA is 14-32 bp, 16-28 bp, or 18-24 bp.
35. The use of item 34, wherein the small RNA comprises the sequence of

(SEQ ID NO: 1)
GTTCAGAGTTCTACAGTCCGA.

36. The use of any one of items 21-35, wherein the delivery comprises treating the compound, a stereoisomer or a pharmaceutically acceptable salt or a combination comprising the compound of Formula (I), a stereoisomer or a pharmaceutically acceptable salt thereof or combination thereof with the nucleic acid, by heating method, reverse evaporation method, direct mixing, repeated freeze-thaw and/or thin film dispersion.
37. The use of item 36, wherein the heating method is conducted at a temperature of about 0° C. to about 100° C., about 25° C. to about 100° C., preferably about 80° C. to about 100° C., for example 4° C., 37° C., 60° C., 80° C. or 100° C.; the reverse evaporation method is conducted at a temperature of about 25° C. to about 70° C., preferably about 55° C. for a heating time of about 0 minutes to about 24 hours, about 5 minutes to about 20 hours, about 5 minutes to about 16 hours, about 5 minutes to about 10 hours, about 5 minutes to about 4 hours, or about 10 hours to about 1 hour, preferably 15 minutes.
38. The use of item 36, wherein the delivery comprises in vitro cell delivery or in vivo subject delivery.
39. The use of item 38, wherein the in vivo subject delivery comprises oral administration, intravenous administration such as injection or infusion, subcutaneous administration, intramuscular administration, intraarterial administration, intraperitoneal administration, intrapulmonary administration, intracerebral and intraspinal administration, intra-articular administration, intrasynovial administration, intrathecal administration, intra-trauma administration, and/or administration via inhalation paths such as intranasal, typically intravenous or subcutaneous administration.
40. A composition or a combination of compounds, comprising the compound of items 1-12, a stereoisomer or a pharmaceutically acceptable salt thereof, or a combination thereof.
41. The composition or the combination of compounds of item 40 for use in delivering a nucleic acid to a cell or a subject.
42. The composition or the combination of compounds of item 41, wherein the nucleic acid is synthetic or purified, therapeutic or non-therapeutic, and/or diagnostic or non-diagnostic, for example selected from RNA or DNA, for example selected from single-stranded or double-stranded or partially double-stranded RNA and DNA;
preferably, when the nucleic acid is therapeutic or diagnostic, the nucleic acid is used to treat or diagnose a disease selected from the group consisting of: inflammatory diseases, pneumonia, myocarditis, acute and chronic gastritis, acute and chronic enteritis, acute and chronic hepatitis, acute and chronic nephritis, dermatitis, encephalitis, lymphitis, conjunctivitis, keratitis, iridocyclitis, otitis media, allergic rhinitis, asthma, pulmonary fibrosis chronic obstructive pulmonary disease, allergic dermatitis, sickle cell disease, multiple sclerosis, systemic lupus erythematosus, lupus nephritis, lung cancer, gastric cancer, colorectal cancer, liver cancer, pancreatic cancer, cervical cancer, breast cancer, leukemia, multiple myeloma, diabetes, and gout.
43. The composition or the combination of compounds of item 42, wherein the RNA is selected from the group consisting of messenger RNA (mRNA), rRNA (ribosomal RNA), tRNA (transfer RNA), heterogeneous nuclear RNA (hnRNA), small nuclear RNA (snRNA), small nucleolar RNA (snoRNA), small cytoplasmic RNA, small RNA, transfer-messenger RNA (tmRNA), telomerase RNA and antisense RNA, preferably small RNA.
44. The composition or the combination of compounds of item 43, wherein the length of the small RNA is 14-32 bp, 16-28 bp, or 18-24 bp.
45. The composition or the combination of compounds of item 44, wherein the small RNA comprises the sequence of GTTCAGAGTTCTACAGTCCGA (SEQ ID NO: 1).
46. The composition or the combination of compounds of any one of items 40-45, wherein the delivery comprises treating the compound, a stereoisomer or a pharmaceutically acceptable salt or a combination comprising the compound of formula (I), a stereoisomer or a pharmaceutically acceptable salt thereof, with nucleic acid by heating method, reverse evaporation method, direct mixing, repeated freeze-thaw and/or thin film dispersion.
47. The composition or the combination of compounds of item 46, wherein the heating method is conducted at a temperature of about 0° C. to about 100° C., about 25° C. to about 100° C., preferably about 80° C. to about 100° C., for example 4° C., 37° C., 60° C., 80° C. or 100° C.; the reverse evaporation method is conducted at a temperature of about 25° C. to about 70° C., preferably about 55° C. for a heating time of about 0 minutes to about 24 hours, about 5 minutes to about 20 hours, about 5 minutes to about 16 hours, about 5 minutes to about 10 hours, about 5 minutes to about 4 hours, or about 10 hours to about 1 hour, preferably 15 minutes.
48. The composition or the combination of compounds of item 46, wherein the delivery comprises in vitro cell delivery or in vivo subject delivery.
49. The composition or the combination of compounds of item 48, wherein the in vivo subject delivery comprises oral administration, intravenous administration such as injection or infusion, subcutaneous administration, intramuscular administration, intraarterial administration, intraperitoneal administration, intrapulmonary administration, intracerebral and intraspinal administration, intra-articular administration, intrasynovial administration, intrathecal administration, intra-trauma administration, and/or administration via inhalation paths such as intranasal, typically intravenous or subcutaneous administration.
50. A kit comprising the compound of items 1-12, a stereoisomer or a pharmaceutically acceptable salt thereof or a combination thereof; preferably, the kit is used for any one of the use above.

### BRIEF DESCRIPTIONS OF THE DRAWINGS

FIG. 1a-e: Different concentrations of sphingosine derivative So-1 deliver single-stranded PGY-sRNA-6 into 293T cells;

FIG. 2a-e: Different concentrations of sphingosine derivative So-3 deliver single-stranded PGY-SRNA-6 into 293T cells;

FIG. 3a-e: Different concentrations of sphingosine derivative So-4 deliver single-stranded PGY-sRNA-6 into 293T cells;

FIG. 4a-e: Different concentrations of sphingosine derivative So-5 deliver single-stranded PGY-s-RNA-6 into 293T cells;

FIG. 5a-e: Different concentrations of sphingosine derivative So-7 deliver single-stranded PGY-sRNA-6 into 293T cells;

FIG. 6a-e: Different concentrations of sphingosine derivative So-8 deliver single-stranded PGY-sRNA-6 into 293T cells;

FIG. 7a-e: Different concentrations of sphingosine derivative So-9 deliver single-stranded PGY-SRNA-6 into 293T cells;

FIG. 8a-e: Different concentrations of sphingosine derivative So-10 deliver single-stranded PGY-sRNA-6 into 293T cells;

FIG. 9a-e: Different concentrations of sphingosine derivative So-11 deliver single-stranded PGY-s-RNA-6 into 293T cells;

FIG. 10a-e: Different concentrations of sphingosine derivative So-12 deliver single-stranded PGY-sRNA-6 into 293T cells;

FIG. 11a-e: Different concentrations of sphingosine derivative So-13 deliver single-stranded PGY-sRNA-6 into 293T cells;

FIG. 12a-e: Different concentrations of sphingosine derivative So-14 deliver single-stranded PGY-sRNA-6 into 293T cells;

FIG. 13a-e: Different concentrations of sphingosine derivative So-15 deliver single-stranded PGY-sRNA-6 into 293T cells;

FIG. 14a-e: Different concentrations of sphingosine derivative So-26 deliver single-stranded PGY-sRNA-6 into 293T cells;

FIG. 15a-e: Different concentrations of sphingosine derivative So-46 deliver single-stranded PGY-sRNA-6 into 293T cells;

FIG. 16a-e: Different concentrations of sphingosine derivative So-49 deliver single-stranded PGY-sRNA-6 into 293T cells;

FIG. 17a-e: Different concentrations of sphingosine derivative So-53 deliver single-stranded PGY-sRNA-6 into 293T cells;

FIG. 18a-e: Different concentrations of sphingosine derivative So-60 deliver single-stranded PGY-sRNA-6 into 293T cells;

FIG. 19a-e: Different concentrations of sphingosine derivative So-61 deliver single-stranded PGY-sRNA-6 into 293T cells;

FIG. 20a-e: Different concentrations of sphingosine derivative So-62 deliver single-stranded PGY-sRNA-6 into 293T cells;

FIG. 21a-e: Different concentrations of sphingosine derivative So-63 deliver single-stranded PGY-sRNA-6 into 293T cells;

FIG. 22a-e: Different concentrations of sphingosine derivative So-64 deliver single-stranded PGY-sRNA-6 into 293T cells;

FIG. 23a-e: Different concentrations of sphingosine derivative So-65 deliver single-stranded PGY-sRNA-6 into 293T cells;

FIG. 24a-e: Different concentrations of sphingosine derivative So-66 deliver single-stranded PGY-sRNA-6 into 293T cells;

FIG. 25a-e: Different concentrations of sphingosine derivative So-67 deliver single-stranded PGY-sRNA-6 into 293T cells;

FIG. 26a-e: Different concentrations of sphingosine derivative So-68 deliver single-stranded PGY-sRNA-6 into 293T cells;

FIG. 27a-e: Different concentrations of sphingosine derivative So-69 deliver single-stranded PGY-sRNA-6 into 293T cells;

FIG. 28a-e: Different concentrations of sphingosine derivative So-70 deliver single-stranded PGY-sRNA-6 into 293T cells;

FIG. 29a-e: Different concentrations of sphingosine derivative So-71 deliver single-stranded PGY-sRNA-6 into 293T cells;

FIG. 30a-e: Different concentrations of sphingosine derivative So-72 deliver single-stranded PGY-s-RNA-6 into 293T cells;

FIG. 31a-e: Different concentrations of sphingosine derivative So-73 deliver single-stranded PGY-sRNA-6 into 293T cells;

FIG. 32a-e: Different concentrations of sphingosine derivative So-74 deliver single-stranded PGY-sRNA-6 into 293T cells;

FIG. 33a-e: Different concentrations of sphingosine derivative So-75 deliver single-stranded PGY-sRNA-6 into 293T cells;

FIG. 34a-c: No. 41+No. 38+sphingosine derivative So-1 mixture delivers single-stranded PGY-sRNA-6 into THP-1 cells;

FIG. 35a-c: No. 41+No. 38+sphingosine derivative So-2 mixture delivers single-stranded PGY-sRNA-6 into THP-1 cells;

FIG. 36a-c: No. 41+No. 38+sphingosine derivative So-3 mixture delivers single-stranded PGY-sRNA-6 into THP-1 cells;

FIG. 37a-c: No. 41+No. 38+sphingosine derivative So-4 mixture delivers single-stranded PGY-sRNA-6 into THP-1 cells;

FIG. 38a-c: No. 41+No. 38+sphingosine derivative So-5 mixture delivers single-stranded PGY-sRNA-6 into THP-1 cells;

FIG. 39a-c: No. 41+No. 38+sphingosine derivative So-6 mixture delivers single-stranded PGY-sRNA-6 into THP-1 cells;

FIG. 40a-c: No. 41+No. 38+-sphingosine derivative So-7 mixture delivers single-stranded PGY-sRNA-6 into TAP-1 cells;

FIG. 41a-c: No. 41+No. 38+sphingosine derivative So-8 mixture delivers single-stranded PGY-sRNA-6 into THP-1 cells;

FIG. 42a-c: No. 41+No. 38+sphingosine derivative So-9 mixture delivers single-stranded PGY-sRNA-6 into THP-1 cells;

FIG. 43a-c: No. 41+No. 38+sphingosine derivative So-10 mixture delivers single-stranded PGY-sRNA-6 into THP-1 cells;

FIG. 44a-c: No. 41+No. 38+sphingosine derivative So-11 mixture delivers single-stranded PGY-sRNA-6 into THP-1 cells;

FIG. 45a-c: No. 41+No. 38+sphingosine derivative So-12 mixture delivers single-stranded PGY-sRNA-6 into THP-1 cells;

FIG. 46a-c: No. 41+No. 38+sphingosine derivative So-13 mixture delivers single-stranded PGY-sRNA-6 into THP-1 cells;

FIG. 47a-c: No. 41+No. 38+sphingosine derivative So-14 mixture delivers single-stranded PGY-sRNA-6 into THP-1 cells;

FIG. 48a-c: No. 41+No. 38+sphingosine derivative So-15 mixture delivers single-stranded PGY-sRNA-6 into THP-1 cells;

FIG. 49a-c: No. 41+No. 38+sphingosine derivative So-45 mixture delivers single-stranded PGY-sRNA-6 into THP-1 cells;

FIG. 50a-c: No. 41+No. 38+sphingosine derivative So-46 mixture delivers single-stranded PGY-sRNA-6 into THP-1 cells;

FIG. 51a-c: No. 41+No. 38+sphingosine derivative So-47 mixture delivers single-stranded PGY-sRNA-6 into THP-1 cells;

FIG. 52a-c: No. 41+No. 38+sphingosine derivative So-48 mixture delivers single-stranded PGY-sRNA-6 into THP-1 cells;

FIG. 53a-c: No. 41+No. 38+sphingosine derivative So-49 mixture delivers single-stranded PGY-sRNA-6 into THP-1 cells;

FIG. 54a-c: No. 41+No. 38+sphingosine derivative So-50 mixture delivers single-stranded PGY-sRNA-6 into THP-1 cells;

FIG. 55a-c: No. 41+No. 38+sphingosine derivative So-51 mixture delivers single-stranded PGY-sRNA-6 into THP-1 cells;

FIG. 56a-c: No. 41+No. 38+sphingosine derivative So-52 mixture delivers single-stranded PGY-sRNA-6 into THP-1 cells;

FIG. 57a-c: No. 41+No. 38+sphingosine derivative So-53 mixture delivers single-stranded PGY-sRNA-6 into THP-1 cells;

FIG. 58a-c: No. 41+No. 38+sphingosine derivative So-54 mixture delivers single-stranded PGY-sRNA-6 into THP-1 cells;

FIG. 59a-c: No. 41+No. 38+sphingosine derivative So-55 mixture delivers single-stranded PGY-sRNA-6 into THP-1 cells;

FIG. 60a-c: No. 41+No. 38+sphingosine derivative So-56 mixture delivers single-stranded PGY-sRNA-6 into THP-1 cells;

FIG. 61a-c: No. 41+No. 38+sphingosine derivative So-57 mixture delivers single-stranded PGY-sRNA-6 into THP-1 cells;

FIG. 62a-c: No. 41+No. 38+sphingosine derivative So-58 mixture delivers single-stranded PGY-sRNA-6 into THP-1 cells;

FIG. 63a-c: No. 41+No. 38+sphingosine derivative So-59 mixture delivers single-stranded PGY-sRNA-6 into THP-1 cells;

FIG. 64a-c: No. 41+No. 38+sphingosine derivative So-60 mixture delivers single-stranded PGY-sRNA-6 into THP-1 cells;

FIG. 65a-c: No. 41+No. 38+sphingosine derivative So-61 mixture delivers single-stranded PGY-sRNA-6 into THP-1 cells;

FIG. 66a-c: No. 41+No. 38+sphingosine derivative So-62 mixture delivers single-stranded PGY-sRNA-6 into THP-1 cells;

FIG. 67a-c: No. 41+No. 38+sphingosine derivative So-63 mixture delivers single-stranded PGY-sRNA-6 into THP-1 cells;

FIG. 68a-c: No. 41+No. 38+sphingosine derivative So-64 mixture delivers single-stranded PGY-sRNA-6 into THP-1 cells;

FIG. 69a-c: No. 41+No. 38+sphingosine derivative So-65 mixture delivers single-stranded PGY-sRNA-6 into THP-1 cells;

FIG. 70a-c: No. 41+No. 38+sphingosine derivative So-66 mixture delivers single-stranded PGY-sRNA-6 into THP-1 cells;

FIG. 71a-c: No. 41+No. 38+sphingosine derivative So-67 mixture delivers single-stranded PGY-sRNA-6 into THP-1 cells;

FIG. 72a-c: No. 41+No. 38+sphingosine derivative So-68 mixture delivers single-stranded PGY-sRNA-6 into THP-1 cells;

FIG. 73a-c: No. 41+No. 38+sphingosine derivative So-69 mixture delivers single-stranded PGY-sRNA-6 into THP-1 cells;

FIG. 74a-c: No. 41+No. 38+sphingosine derivative So-70 mixture delivers single-stranded PGY-sRNA-6 into THP-1 cells;

FIG. 75a-c: No. 41+No. 38+sphingosine derivative So-71 mixture delivers single-stranded PGY-sRNA-6 into THP-1 cells;

FIG. 76a-c: No. 41+No. 38+sphingosine derivative So-72 mixture delivers single-stranded PGY-sRNA-6 into THP-1 cells;

FIG. 77a-c: No. 41+No. 38+sphingosine derivative So-73 mixture delivers single-stranded PGY-sRNA-6 into THP-1 cells;

FIG. 78a-c: No. 41+No. 38+sphingosine derivative So-74 mixture delivers single-stranded PGY-sRNA-6 into THP-1 cells;

FIG. 79a-c: No. 41+No. 38+sphingosine derivative So-75 mixture delivers single-stranded PGY-sRNA-6 into THP-1 cells;

FIG. 80a-c: No. 41+No. 38+No. 48+sphingosine derivative So-42 mixture delivers single-stranded PGY-sRNA-6 into THP-1 cells;

FIG. 81a-c: No. 41+No. 38+No. 48+sphingosine derivative So-43 mixture delivers single-stranded PGY-sRNA-6 into THP-1 cells;

FIG. 82a-c: No. 41+No. 38+No. 48+sphingosine derivative So-44 mixture delivers single-stranded PGY-sRNA-6 into THP-1 cells;

FIG. 83a-c: No. 41+No. 38+No. 48+sphingosine derivative So-45 mixture delivers single-stranded PGY-sRNA-6 into THP-1 cells;

FIG. 84a-c: No. 41+No. 38+No. 48+sphingosine derivative So-46 mixture delivers single-stranded PGY-sRNA-6 into THP-1 cells;

FIG. 85a-c: No. 41+No. 38+No. 48+sphingosine derivative So-47 mixture delivers single-stranded PGY-sRNA-6 into THP-1 cells;

FIG. 86a-c: No. 41+No. 38+No. 48+sphingosine derivative So-52 mixture delivers single-stranded PGY-sRNA-6 into THP-1 cells;

FIG. 87a-c: No. 41+No. 38+No. 48+sphingosine derivative So-56 mixture delivers single-stranded PGY-sRNA-6 into THP-1 cells;

FIG. 88a-c: No. 41+No. 38+No. 48+sphingosine derivative So-57 mixture delivers single-stranded PGY-sRNA-6 into THP-1 cells;

FIG. 89a-c: No. 41+No. 38+No. 48+sphingosine derivative So-58 mixture delivers single-stranded PGY-sRNA-6 into THP-1 cells;

FIG. 90a-c: No. 41+No. 38+No. 48+sphingosine derivative So-59 mixture delivers single-stranded PGY-sRNA-6 into THP-1 cells;

FIG. 91a-c: No. 41+No. 38+No. 48+sphingosine derivative So-60 mixture delivers single-stranded PGY-sRNA-6 into THP-1 cells;

FIG. 92a-c: No. 41+No. 38+No. 48+sphingosine derivative So-61 mixture delivers single-stranded PGY-sRNA-6 into THP-1 cells;

FIG. 93a-c: No. 41+No. 38+No. 48+sphingosine derivative So-62 mixture delivers single-stranded PGY-sRNA-6 into THP-1 cells;

FIG. 94a-c: No. 41+No. 38+No. 48+sphingosine derivative So-63 mixture delivers single-stranded PGY-sRNA-6 into THP-1 cells;

FIG. 95a-c: No. 41+No. 38+No. 48+sphingosine derivative So-64 mixture delivers single-stranded PGY-sRNA-6 into THP-1 cells;

FIG. 96a-c: No. 41+No. 38+No. 48+sphingosine derivative So-67 mixture delivers single-stranded PGY-sRNA-6 into THP-1 cells;

FIG. 97a-c: No. 41+No. 38+No. 48+sphingosine derivative So-68 mixture delivers single-stranded PGY-sRNA-6 into THP-1 cells;

FIG. 98a-c: No. 41+No. 38+No. 48+sphingosine derivative So-69 mixture delivers single-stranded PGY-sRNA-6 into THP-1 cells;

FIG. 99a-c: No. 41+No. 38+No. 48+sphingosine derivative So-70 mixture delivers single-stranded PGY-sRNA-6 into THP-1 cells;

FIG. 100: Results of lipid combination delivering single-stranded PGY-sRNA-6 into 293T;

FIG. 101: Results of lipid combination delivering single-stranded PGY-sRNA-6 into 293T;

FIG. 102: Results of lipid combination delivering single-stranded PGY-sRNA-6 into 293T;

FIG. 103: Results of lipid combination delivering single-stranded PGY-SRNA-6 into 293T;

FIG. 104: Results of lipid combination delivering single-stranded PGY-sRNA-6 into 293T.

### DETAILED DESCRIPTION OF THE INVENTION

### Definition of terms

The term as used herein may have a single dash “-” (or horizontal line) or a double dash “=” in front of and/or behind it to indicate the bond level of the bond between the mentioned substituent and its parent moiety; a single dash “-” (or horizontal line) refers to a single bond, and a double dash refers to a double bond; in the absence of single or double dash, it is understood that a single bond is formed between the substituent and its parent moiety; in addition, the substituent is to be construed “from left to right” unless otherwise indicated.

A dashed line (“-”) that is not between two letters or symbols is used to represent the linking points of substituents. For example, —C(O)NH₂ is linked through a carbon atom. The dashed line before or after a chemical group is for convenience. The chemical group can be depicted with or without one or more dashed lines without losing its usual meaning. The wavy line drawn through the lines in the structure represents the linking points of the groups.

When a range of values is listed, it is intended to include every value and subrange within the range. For example, “C₁₋₆alkyl” is intended to include C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆alkyl groups.

The term “alkyl” as used herein refers to a straight or branched saturated hydrocarbon chain. As described herein, an alkyl group has 1 to 24 carbon atoms (i.e., C₁₋₂₄alkyl), 1 to 20 carbon atoms (i.e., C₁₋₂₀alkyl), 1 to 8 carbon atoms (i.e., C₁₋₈alkyl), 1 to 6 carbon atoms (i.e., C₁₋₆alkyl), or 1 to 4 carbon atoms (i.e., C₁₋₄alkyl). In one embodiment, the alkyl group is a C₁₋₆alkyl group. In one embodiment, the alkyl group is a C₁₄₋₂₀alkyl group. In one embodiment, the alkyl group is a straight C₁₄, C₁₅, C₁₆, C₁₇, C₁₈, C₁₉, C₂₀alkyl group.

The term “alkenyl” as used herein refers to an aliphatic hydrocarbon chain having specified number of carbon atoms and containing at least one carbon-carbon double bond. As described herein, an alkenyl group has 2 to 24 carbon atoms (i.e., C₂₋₂₄alkenyl), 2 to 20 carbon atoms (i.e., C₂₋₂₀alkenyl), 2 to 8 carbon atoms (i.e., C₂₋₈alkenyl), 2 to 6 carbon atoms (i.e., C₂₋₆alkenyl), or 2 to 4 carbon atoms (i.e., C₂₋₄alkenyl). In one embodiment, the alkenyl group is a C₁₄₋₂₀alkenyl group. In one embodiment, the alkenyl group is a C₁₄, C₁₅, C₁₆, C₁₇, C₁₈, C₁₉, C₂₀alkenyl group.

As used herein, the term “acyl” refers to group —CO—.

As used herein, the term “phosphate ester” refers to group

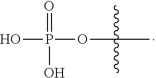 [SMILES: *OP(=O)(O)O]

As used herein, the term “biotin acyl” refers to group

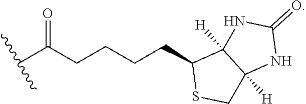 [SMILES: *C(=O)CCCC[C@@H]1SC[C@@H]2NC(=O)N[C@@H]21]

As used herein, the term “glycosyl” refers to a monovalent substituent obtained by removing the hemiacetal hydroxyl group from the cyclic form of a monosaccharide. For example, the term “1-β-D-glucosyl” refers to group

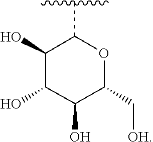 [SMILES: *[C@@H]1O[C@H](CO)[C@@H](O)[C@H](O)[C@H]1O]

Examples of glycosyl include, but are not limited to, glucosyl, lactosyl, galactosyl, mannosyl, fructosyl, and sorbosyl. In one embodiment, the glycosyl is β-D-glucosyl.

The term “pharmaceutically acceptable salt” refers to the salt, within the scope of reasonable medical judgment, suitable for use in contact with tissues of human and lower animals without improper toxicity, stimulation, allergic reactions, etc. and commensurate with a reasonable benefit/risk ratio. Pharmaceutically acceptable salts are well known in the art. For example, Berge et al. described pharmaceutically acceptable salts in detail in J. Pharmaceutical Sciences, 1977, 66, pages 1-19, which is incorporated herein by reference. Pharmaceutically acceptable salts of the compounds of the present application include those derived from suitable inorganic and organic acids and bases. Examples of pharmaceutically acceptable non-toxic acid addition salts are salts formed from amino with inorganic acids (such as hydrochloric acid, hydrobromic acid, phosphoric acid, sulfuric acid, and perchloric acid) or with organic acids (such as acetic acid, oxalic acid, maleic acid, tartaric acid, citric acid, succinic acid, or malonic acid), or salts formed by using other methods known in the art (for example, ion exchange methods). Other pharmaceutically acceptable salts include adipate, alginate, ascorbate, aspartate, benzenesulfonate, benzoate, bisulfate, borate, butyrate, camphorate, camphor sulfonate, citrate, cyclopentane propionate, digluconate, lauryl sulfate, ethane sulfonate, formate, fumarate, gluceptate, glycerophosphate, gluconate, hemisulfate, enanthate, caproate, hydroiodide, 2-hydroxy-ethane sulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, picrate, pivalate, propionate, stearate, succinate, sulfate, methyl sulfate, tartrate, thiocyanate, p-toluenesulfonate, undecanoate, valerate, etc. Salts derived from appropriate bases include alkali metal salts, alkaline-earth metal salts, ammonium salts, and N⁺(C₁₋₄alkyl)₄⁻salts. Representative alkali metal or alkaline-earth metal salts include sodium salt, lithium salt, potassium salt, calcium salt, magnesium salt and the like. Where appropriate, other pharmaceutically acceptable salts include non-toxic ammonium, quaternary ammonium, and amine cations formed using counterions such as halogen, hydroxide, carboxylate, sulfate, phosphate, nitrate, lower alkylsulfonate and arylsulphonate.

The term reverse evaporation method as described herein refers to adding an aqueous solution of nucleic acid to a solution of lipid in an organic solvent, ultrasonicating, evaporating to remove the organic solvent, and then hydrating to obtain a lipid nucleic acid mixture.

The term “boiling method” (also refers to “heating method”) as described herein refers to adding an organic solvent solution of lipid to an aqueous solution of nucleic acid and boiling at about 100° C. for 30 minutes to obtain a lipid nucleic acid mixture. The method is not limited to heating by boiling, and other means of heating or raising temperature known in the art can also be used.

Reverse evaporation method and boiling method are carried out under controlled temperature and mixing conditions. Suitable processing times, and temperatures can be readily determined by a person skilled in the art. For example, the temperature of reverse evaporation method is ranged preferably from about 25° C. to about 70° C., more preferably from about 30° C. to about 65° C., and more preferably from about 40° C. to about 60° C., especially about 55° C. The temperature of boiling method is ranged preferably from about 25° C. to about 100° C., more preferably from about 50° C. to about 100° C., and more preferably from about 95° C. to about 10° C., especially, preferably from about 80° C. to 100° C.

The nucleic acid as described herein comprises DNA and RNA, preferably small RNA, for example, the small RNA having a length of 14-32 bp, 16-28 bp, 18-24 bp, and particularly, a length of 14, 15, 16, 17, 18, 19, 20, 21, 22, 23, 24, 25, 26, 27, 28, 29, 30, 31, 32 bp.

In the present application, a nucleic acid can be delivered to a cell or a subject by mixing compounds or combination or composition containing one or more of the compounds and the nucleic acid. In one embodiment, the compound has a structure of Formula (I), a stereoisomer thereof or a pharmaceutical acceptable salt thereof,

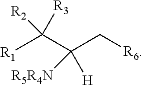

In one embodiment, the substituents of Formula (I) are as defined above. The compound may be a compound in Table 1. The combination or composition comprising the compound may include any one or more of the compounds in Table 1. Those skilled in the art can add or delete one or more compounds in the combination or composition as needed.

In one embodiment, the combination or composition herein is a combination comprising any one or more of No. 41, No. 38, No. 48. In one embodiment, a combination comprising any one or more of No. 41, No. 38, No. 48 and any one or more of the compound selecting from item 11. In one embodiment, a combination comprising the following: No. 41+No. 38+sphingosine derivative So-1; No. 41+No. 38+sphingosine derivative So-2; No. 41+No. 38+sphingosine derivative So-3; No. 41+No. 38+sphingosine derivative So-4; No. 41+No. 38+sphingosine derivative So-5; No. 41+No. 38+sphingosine derivative So-6; No. 41+No. 38+sphingosine derivative So-7; No. 41+No. 38+sphingosine derivative So-8; No. 41+No. 38+sphingosine derivative So-9; No. 41+No. 38+sphingosine derivative So-10; No. 41+No. 38+sphingosine derivative So-11; No. 41+No. 38+sphingosine derivative So-12; No. 41+No. 38+sphingosine derivative So-13; No. 41+No. 38+sphingosine derivative So-14; No. 41+No. 38+sphingosine derivative So-15; No. 41+No. 38+sphingosine derivative So-45; No. 41+No. 38+sphingosine derivative So-46; No. 41+No. 38+sphingosine derivative So-47; No. 41+No. 38+sphingosine derivative So-48; No. 41+No. 38+sphingosine derivative So-49; No. 41+No. 38+sphingosine derivative So-50; No. 41+No. 38+sphingosine derivative So-51; No. 41+No. 38+sphingosine derivative So-52; No. 41+No. 38+sphingosine derivative So-53; No. 41+No. 38+sphingosine derivative So-54; No. 41+No. 38+sphingosine derivative So-55; No. 41+No. 38+sphingosine derivative So-56; No. 41+No. 38+sphingosine derivative So-57; No. 41+No. 38+sphingosine derivative So-58; No. 41+No. 38+sphingosine derivative So-59; No. 41+No. 38+sphingosine derivative So-60; No. 41+No. 38+sphingosine derivative So-61; No. 41+No. 38+sphingosine derivative So-62; No. 41+No. 38+sphingosine derivative So-63; No. 41+No. 38+sphingosine derivative So-64; No. 41+No. 38+sphingosine derivative So-65; No. 41+No. 38+sphingosine derivative So-66; No. 41+No. 38+sphingosine derivative So-67; No. 41+No. 38+sphingosine derivative So-68; No. 41+No. 38+sphingosine derivative So-69; No. 41+No. 38+sphingosine derivative So-70; No. 41+No. 38+sphingosine derivative So-71; No. 41+No. 38+sphingosine derivative So-72; No. 41+No. 38+sphingosine derivative So-73; No. 41+No. 38+sphingosine derivative So-74; No. 41+No. 38+sphingosine derivative So-75; No. 41+No. 38+No. 48+sphingosine derivative So-42; No. 41+No. 38+No. 48+sphingosine derivative So-43; No. 41+No. 38+No. 48+sphingosine derivative So-44; No. 41+No. 38+No. 48+sphingosine derivative So-45; No. 41+No. 38+No. 48+sphingosine derivative So-46; No. 41+No. 38+No. 48+sphingosine derivative So-47; No. 41+No. 38+No. 48+sphingosine derivative So-52; No. 41+No. 38+No. 48+sphingosine derivative So-56; No. 41+No. 38+No. 48+sphingosine derivative So-57; No. 41+No. 38+No. 48+sphingosine derivative So-58; No. 41+No. 38+No. 48+sphingosine derivative So-59; No. 41+No. 38+No. 48+sphingosine derivative So-60; No. 41+No. 38+No. 48+sphingosine derivative So-61; No. 41+No. 38+No. 48+sphingosine derivative So-62; No. 41+No. 38+No. 48+sphingosine derivative So-63; No. 41+No. 38+No. 48+sphingosine derivative So-64; No. 41+No. 38+No. 48+sphingosine derivative So-67; No. 41+No. 38+No. 48+sphingosine derivative So-68; No. 41+No. 38+No. 48+sphingosine derivative So-69; No. 41+No. 38+No. 48+sphingosine derivative So-70; No. 41+No. 38+any one or more of sphingosine derivative So-23, 25, 26, 27, 29, 30, 31, 32, 33, 34, 35, 36, 37, 38, 40, 41, 42, 43, 44, 70, 71 or 73. Those skilled in the art can select the appropriate concentration and use volume. Preferably, the compounds above are used at concentrations as shown in Table 1. The ratio of the compounds above is 0.1-10:0.1-10, 0.2-9:0.2-9, 0.3-8:0.3-8, 0.4-7:0.4-7, 0.5-6:0.5-6, 0.6-5:0.6-5, 0.7-4:0.7-4, 0.8-3:0.8-3, 0.9-2:0.9-2; more preferably 1:1, or any ratio between them. Those skilled in the art can appropriately adjust the ratio of various compounds according to the concentration of the mother liquor of the compounds. In addition, the present application proved that a specific lipid combination can effectively promote the delivery of nucleic acids, and the effect is better than that of a single lipid.

In one embodiment, the nucleic acid is synthetic or purified, therapeutic or non-therapeutic, and/or diagnostic or non-diagnostic, for example selected from RNA or DNA, for example selected from single-stranded or double-stranded or partially double-stranded RNA or DNA. When the nucleic acid is therapeutic or diagnostic, the nucleic acid is used to treat or diagnose a disease selected from the group consisting of: inflammatory diseases, pneumonia, myocarditis, acute and chronic gastritis, acute and chronic enteritis, acute and chronic hepatitis, acute and chronic nephritis, dermatitis, encephalitis, lymphitis, conjunctivitis, keratitis, iridocyclitis, otitis media, allergic rhinitis, asthma, pulmonary fibrosis, chronic obstructive pulmonary disease, allergic dermatitis, sickle cell disease, multiple sclerosis, systemic lupus erythematosus, lupus nephritis, lung cancer, gastric cancer, colorectal cancer, liver cancer, pancreatic cancer, cervical cancer, breast cancer, leukemia, multiple myeloma, diabetes, and gout. Those skilled in the art can select a suitable nucleic acid according to the specific situation. For example, the RNA can be messenger RNA (mRNA), rRNA (ribosomal RNA), tRNA (transfer RNA), heterogeneous nuclear RNA (hnRNA), small nuclear RNA (snRNA), small nucleolar RNA (snoRNA), small cytoplasmic RNA, small RNA, transfer-messenger RNA (tmRNA), telomerase RNA and antisense RNA, preferably small RNA.

In one embodiment, the delivery comprises treating the compound, a stereoisomer thereof or a pharmaceutically acceptable salt thereof or a combination comprising them by heating method, reverse evaporation method, direct mixing, repeated freeze-thaw and/or thin film dispersion. Those skilled in the art can select a suitable method for delivery. In one embodiment, the heating method is conducted at a temperature of about 0° C. to about 100° C., about 25° C. to about 100° C., preferably about 80° C. to about 100° C., for example 4° C., 37° C., 60° C., 80° C. or 100° C.; the reverse evaporation method is conducted at a temperature of about 25° C. to about 70° C., preferably about 55° C. for a heating time of about 0 minutes to about 24 hours, about 5 minutes to about 20 hours, about 5 minutes to about 16 hours, about 5 minutes to about 10 hours, about 5 minutes to about 4 hours, or about 10 hours to about 1 hour, preferably 15 minutes.

In one embodiment, the nucleic acid is treated with the compound, and the treated mixture can be directly administered to the subject via oral administration. In addition, the subject can also be administered by other means, for example, intravenous administration such as injection or infusion, subcutaneous administration. intramuscular administration, intraarterial administration, intraperitoneal administration, intrapulmonary administration, intracerebral and intraspinal administration, intra-articular administration, intrasynovial administration, intrathecal administration, intra-trauma administration, and/or administration via inhalation paths such as intranasal, typically intravenous or subcutaneous administration.

The invention also provides combinations, compositions or kits of compounds, which comprise any compound described herein, such as any one or more of the compounds in Table 1. Those skilled in the art can add other compounds to the combinations, compositions or kits of compounds as needed, as long as the function of delivering nucleic acid is retained.

TABLE 1
Molecular structure information of single lipid

| | | | | | | Con- |
|---|---|---|---|---|---|---|
| | | | | | | centra- |
| | | | | | | tion in |
| | | | | | | chloro- |
| | | | | | | form |
| | | Catalog | | | | solution/ |
| No. | Brand | No. | Abbreviation | IUPAC Name | Molecular structure | (mg/mL) |
| So-1 | Cayman | CAC- 24427-5 | C17 dihydro Ceramide (d18:0/17:0) | CAS No 1388156-40-8 N-heptadecanoyl-D- erythro- Dihydrosphingosine | 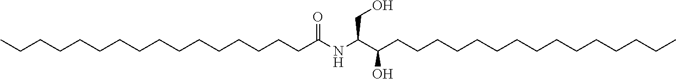 [SMILES: CCCCCCCCCCCCCCCCC(=O)N[C@@H](CO)[C@H](O)CCCCCCCCCCCCCCC] | 5 |
| So-2 | Cayman | CAC- 9002921-5 | Sphingosine-1- phosphate (d16:1) | CAS No 709026-60-8 C16 Sphingosine-1- phosphate | 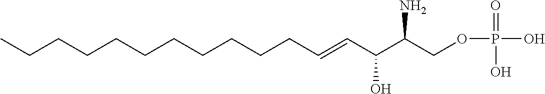 [SMILES: CCCCCCCCCCC/C=C/[C@@H](O)[C@@H](N)COP(=O)(O)O] | 5 |
| So-3 | Cayman | CAC- 9000415-5 | C6 Biotin Ceramide (d18:1/6:0) | N-hexanoyl-biotin-D- erythro-Sphingosine | 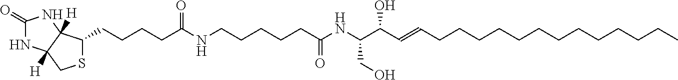 [SMILES: CCCCCCCCCCCCC/C=C/[C@@H](O)[C@H](CO)NC(=O)CCCCCNC(=O)CCCC[C@@H]1SC[C@@H]2NC(=O)N[C@@H]21] | 5 |
| So-4 | Cayman | CAC- 62575-5 | N,N- Dimethylsphingosine (d18:1) | CAS No 119567-63-4 | 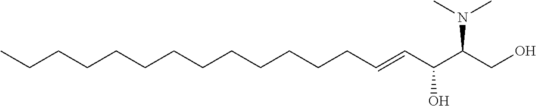 [SMILES: CCCCCCCCCCCCC/C=C/[C@@H](O)[C@H](CO)N(C)C] | 5 |
| So-5 | Cayman | CAC- 62570-5 | Sphingosine-1- phosphate (d18:1) | CAS No 26993-30-6 Sphingosine-1-Phosphoric Acid | 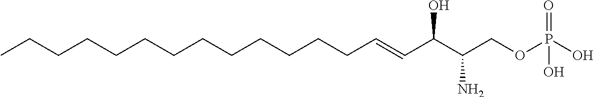 [SMILES: CCCCCCCCCCCCC/C=C/[C@@H](O)[C@@H](N)COP(=O)(O)O] | 5 |
| So-6 | Cayman | CAC- 24867-1 | Lactosylsphingosine (d18:1) (synthetic) | CAS No 109785-20-8 Lyso-Lactosylceramide (synthetic) | 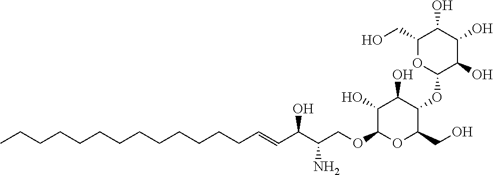 [SMILES: CCCCCCCCCCCCC/C=C/[C@@H](O)[C@@H](N)CO[C@@H]1O[C@H](CO)[C@@H](O[C@@H]2O[C@H](CO)[C@H](O)[C@H](O)[C@H]2O)[C@H](O)[C@H]1O] | 1 |
| So-7 | Cayman | CAC- 24515-1 | 1-Deoxysphingosine (m18:1(14Z)) | CAS No 2190487-94-4 14Z-1-Deoxysphingosine (d18:1) | 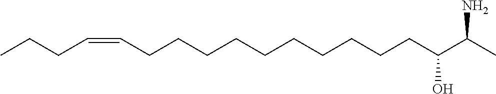 [SMILES: CCC/C=C\CCCCCCCCCC[C@@H](O)[C@H](C)N] | 5 |
| So-8 | Cayman | CAC- 24380-5 | 3-keto Sphinganine (d18:0) (hydrochloride) | CAS No 35299-94-6 3-keto Dihydrosphingosine | 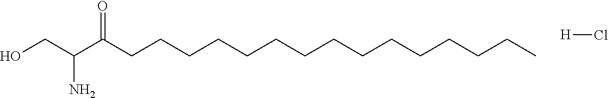 | 5 |
| So-9 | Cayman | CAC- 24379-10 | C18 dihydro Ceramide (d18:0/18:0) | CAS No 2304-80-5 N-octadecanoyl-D-erythro- Dihydrosphingosine | 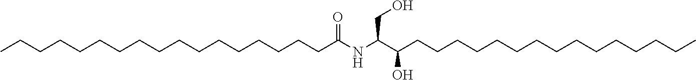 [SMILES: CCCCCCCCCCCCCCCCCC(=O)N[C@@H](CO)[C@H](O)CCCCCCCCCCCCCCC] | 10 |
| So-10 | Cayman | CAC- 24369-5 | C16 dihydro Ceramide (d18:0/16:0) | CAS No 5966-29-0 N-hexadecanoyl-D-erythro- Dihydrosphingosine | 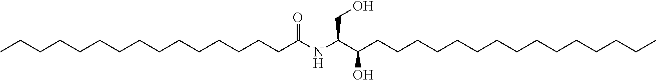 [SMILES: CCCCCCCCCCCCCCCC(=O)N[C@@H](CO)[C@H](O)CCCCCCCCCCCCCCC] | 5 |
| So-11 | Cayman | CAC- 22504-5 | N,N-dimethyl Sphinganine (d18:0) | CAS No 17267-46-8 N,N-dimethyl-D-erythro- Dihydrosphingosine | 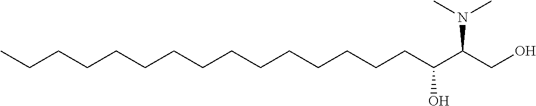 [SMILES: CCCCCCCCCCCCCCC[C@@H](O)[C@H](CO)N(C)C] | 5 |
| So-12 | Cayman | CAC- 24358-5 | C8 dihydro Ceramide (d18:0/8:0) | CAS No 145774-33-0 N-octanoyl-D-erythro- Dihydrosphingosine | 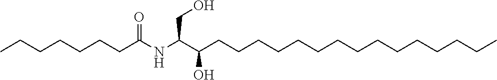 [SMILES: CCCCCCCCCCCCCCC[C@@H](O)[C@H](CO)NC(=O)CCCCCCC] | 5 |
| So-13 | Cayman | CAC- 22530-5 | C12 Ceramide (d18:1/12:0) | CAS No 74713-60-3 N-Lauroyl-D-erythro- Sphingosine | 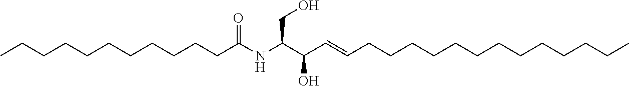 [SMILES: CCCCCCCCCCCCC/C=C/[C@@H](O)[C@H](CO)NC(=O)CCCCCCCCCCC] | 5 |
| So-14 | Cayman | CAC- 22688-5 | C30(ω-hydroxy) Ceramide (d18:1/30:0) | CAS No 457100-08-2 ω-hydroxy-C30 Sphingosine (d18:1/30:0) | 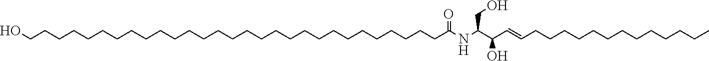 [SMILES: CCCCCCCCCCCCC/C=C/[C@@H](O)[C@H](CO)NC(=O)CCCCCCCCCCCCCCCCCCCCCCCCCCCCCO] | 5 |
| So-15 | Cayman | CAC- 22501-5 | C17 Sphinganine-1- phosphate (d17:0) | CAS No 474923-29-0 dihydro-D-erythro- Sphingosine-C17-1- phosphate | 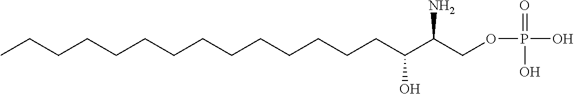 [SMILES: CCCCCCCCCCCCCC[C@@H](O)[C@@H](N)COP(=O)(O)O] | 5 |
| So-16 | Cayman | CAC- 22827-5 | C24 Phytosphingosine (t18:0/24:0) | CAS No 34437-74-6 N-Tetracosanoyl Phytosphingosine | 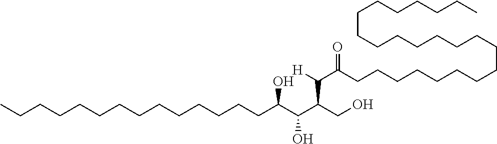 [SMILES: CCCCCCCCCCCCCCCCCCCCCCCC(=O)C[C@@H](CO)[C@H](O)[C@H](O)CCCCCCCCCCCCCC] | 5 |
| So-17 | Cayman | CAC- 20338-5 | Galactosylsphingosine (d18:1) | CAS No 2238-90-6 Galactosylsphingosine | 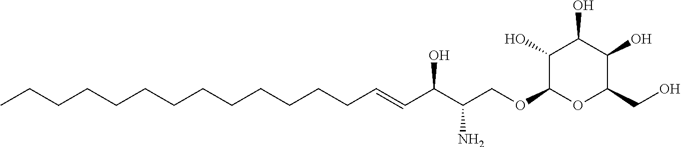 [SMILES: CCCCCCCCCCCCC/C=C/[C@@H](O)[C@@H](N)CO[C@@H]1O[C@H](CO)[C@H](O)[C@H](O)[C@H]1O] | 5 |
| So-18 | Cayman | CAC- 19556-5 | C18 Ceramide (d18:1/18:0) | CAS No 2304-81-6 N-Stearoyl-D-Sphingosine | 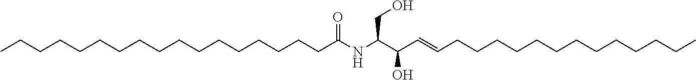 [SMILES: CCCCCCCCCCCCC/C=C/[C@@H](O)[C@H](CO)NC(=O)CCCCCCCCCCCCCCCCC] | 5 |
| So-19 | Cayman | CAC- 20217-5 | Phytosphingosine | CAS No 554-62-1 D-ribo Phytosphingosine | 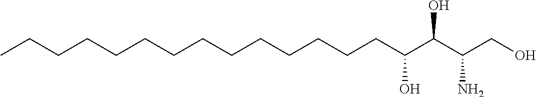 [SMILES: CCCCCCCCCCCCCC[C@@H](O)[C@@H](O)[C@@H](N)CO] | 5 |
| So-20 | Cayman | CAC- 10724-5 | C20 Ceramide (d18:1/20:0) | CAS No 7344-02-7 N-arachidoyl-D-erythro- Sphingosine | 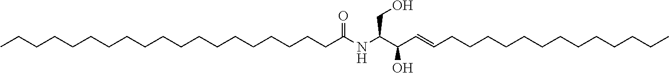 [SMILES: CCCCCCCCCCCCC/C=C/[C@@H](O)[C@H](CO)NC(=O)CCCCCCCCCCCCCCCCCCC] | 5 |
| So-21 | Cayman | CAC- 22500-5 | Sphinganine-1- phosphate (d18:0) | CAS No 19794-97-9 dihydro-D-erythro- Sphingosine-1-phosphate | 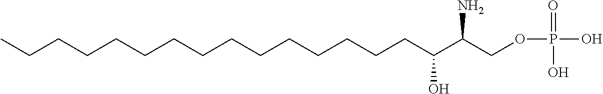 [SMILES: CCCCCCCCCCCCCCC[C@@H](O)[C@@H](N)COP(=O)(O)O] | 5 |
| So-23 | Cayman | CAC- 10681-5 | C16 Ceramidc (d18:1/16:0) | CAS No 24696-26-2 N-Palmitoyl-D-erythro- Sphingosine | 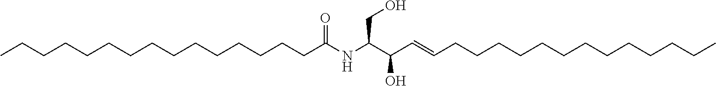 [SMILES: CCCCCCCCCCCCC/C=C/[C@@H](O)[C@H](CO)NC(=O)CCCCCCCCCCCCCCC] | 5 |
| So-25 | Cayman | CAC- 10007901- 5 | Sphingosinc (d15:1) | CAS No 86555-28-4 D-erythro-Sphingosine C- 15 | 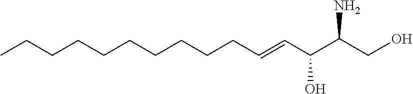 [SMILES: CCCCCCCCCC/C=C/[C@@H](O)[C@@H](N)CO] | 5 |
| So-26 | Cayman | CAC- 10007907- 10 | Sphingosine (d18:1) | CAS No 123-78-4 D-erythro-Sphingosine C- 18 | 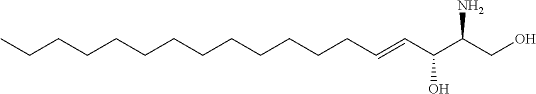 [SMILES: CCCCCCCCCCCCC/C=C/[C@@H](O)[C@@H](N)CO] | 10 |
| So-27 | Avanti | 860660P- 5MG | Sphingosine (D20:1) | CAS: 6918-49-6 D-erythro-sphingosine (C20 base) | 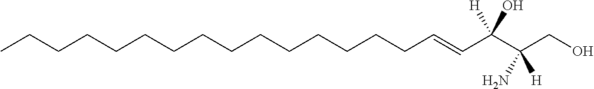 [SMILES: CCCCCCCCCCCCCCC/C=C/[C@@H](O)[C@@H](N)CO] | 5 |
| So-29 | Avanti | 860497P- 5MG | TRIMETHYL SPHINGOSINE (D18:1) | CAS: 133561-52-1 N,N,N-trimethyl-D- erythro-sphingosine (methyl sulfate salt) | 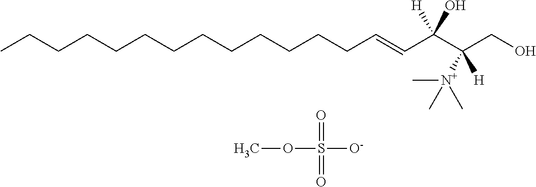 | 5 |
| So-30 | Avanti | 860601P- 5MG | DIMETHYL SPHINGOSINE-1- PHOSPHATE (D18:1) | CAS: 474943-83-4 N,N-dimethyl-D-erythro- sphingosine-1-phosphate (ammonium salt) | 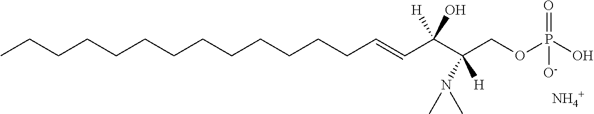 [SMILES: CCCCCCCCCCCCC/C=C/[C@@H](O)[C@H](COP(=O)([O-])O)N(C)C ; [NH4+]] | 5 |
| So-31 | Avanti | 860643P- 5MG | TRIMETHYL SPHINGOSINE (D17:1) | CAS: 474943-94-7 N,N,N-trimethyl-D- erythro-sphingosine (C17 base) (methyl sulfate salt) | 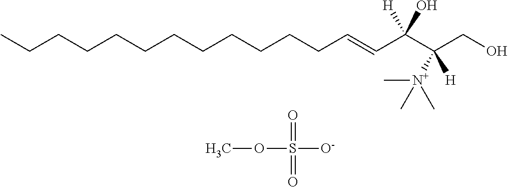 | 5 |
| So-32 | Avanti | 860536P- 5MG | SPHINGANINE-1- PHOSPHATE (D18:0) | CAS: 19794-97-9 D-erythro-sphinganine-1- phosphate | 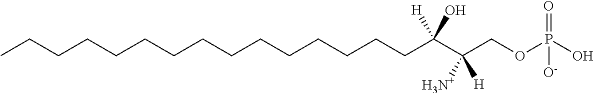 [SMILES: CCCCCCCCCCCCCCC[C@@H](O)[C@@H]([NH3+])COP(=O)([O-])O] | 5 |
| So-33 | Avanti | 860641P- 5MG | SPHINGOSINE-1- PHOSPHATE (D17:1) | CAS: 474923-27-8 D-erythro-sphingosine-1- phosphate (C17 base) | 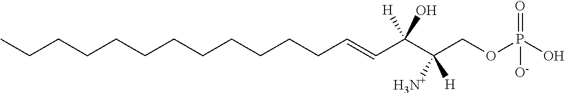 [SMILES: CCCCCCCCCCCC/C=C/[C@@H](O)[C@@H]([NH3+])COP(=O)([O-])O] | 5 |
| So-34 | Avanti | 860655P- 1MG | SPHINGANINE-1- PHOSPHATE (D17:0) | CAS: 474923-29-0 D-erythro-sphinganine-1- phosphate (C17 base) | 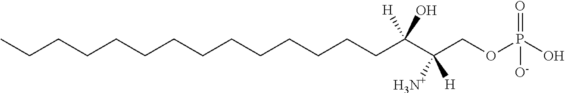 [SMILES: CCCCCCCCCCCCCC[C@@H](O)[C@@H]([NH3+])COP(=O)([O-])O] | 5 |
| So-35 | Avanti | 860662P- 1MG | SPHINGOSINE-1- PHOSPHATE (D20:1) | CAS: 799812-75-2 D-erythro-sphingosine-1- phosphate (C20 base) | 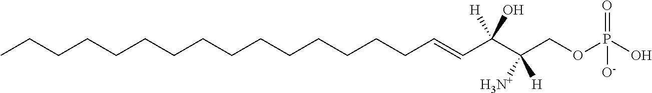 [SMILES: CCCCCCCCCCCCCCC/C=C/[C@@H](O)[C@@H]([NH3+])COP(=O)([O-])O] | 5 |
| So-36 | Avanti | 860481P- 1MG | N-12:0-1-DEOXY- SPHINGANINE | CAS: 1246298-40-7 N-dodecanoyl-1- deoxysphinganine | 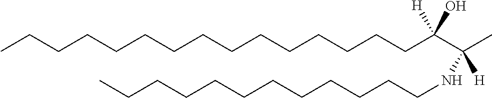 [SMILES: CCCCCCCCCCCCCCC[C@@H](O)[C@H](C)NCCCCCCCCCCCC] | 5 |
| So-37 | Avanti | 860489P- 5MG | L-THREO- SPHINGOSINE (D18:1) | CAS: 25695-95-8 (2S,3S,4E)-2- aminooctadec-4-ene-1,3- diol | 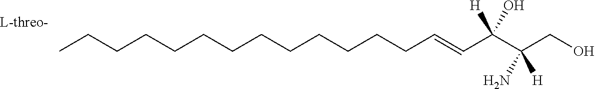 [SMILES: CCCCCCCCCCCCC/C=C/[C@H](O)[C@@H](N)CO] | 5 |
| So-38 | Avanti | 860665P- 1MG | 4E 14Z- SPHINGADIENE | CAS: 25696-03-1 (2S,3R,4E,14Z)-2- aminooctadec-4,14-diene- 1,3-diol | 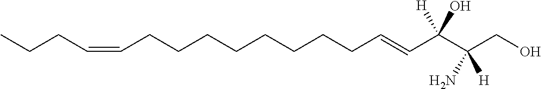 [SMILES: CCC/C=C\CCCCCCCC/C=C/[C@@H](O)[C@@H](N)CO] | 5 |
| So-40 | Avanti | 860675P- 1mg | Sphinganine-1- Phosphate (d20:0) | CAS: 436846-91-2 D-erythro-sphinganine-1- phosphate (C20 base) | 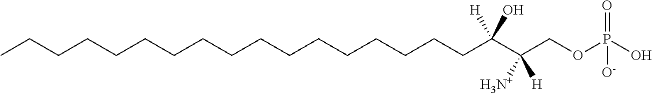 [SMILES: CCCCCCCCCCCCCCCCC[C@@H](O)[C@@H]([NH3+])COP(=O)([O-])O] | 5 |
| So-41 | Avanti | 860640P- 5MG | Sphingosine (d17:1) | D-erythro-sphingosine (C17 base) | 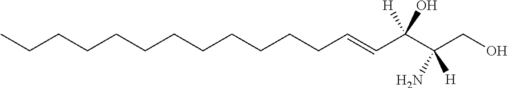 [SMILES: CCCCCCCCCCCC/C=C/[C@@H](O)[C@@H](N)CO] | 5 |
| So-42 | Cayman | 22532 | C17 Ceramide (d18:1/17:0) | CAS No 67492-16-4 N-heptadecanoyl-D- erythro-Sphingosine | 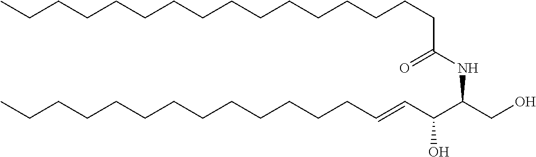 [SMILES: CCCCCCCCCCCCC/C=C/[C@@H](O)[C@H](CO)NC(=O)CCCCCCCCCCCCCCCC] | 5 |
| So-43 | Cayman | 22531 | C14 Ceramide (d18:1/14:0) | CAS No 34227-72-0 Ceramide (d18:1/14:0) | 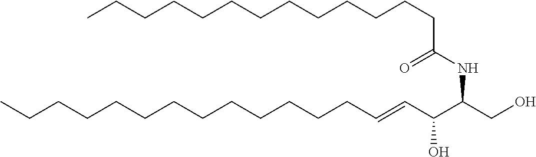 [SMILES: CCCCCCCCCCCCC/C=C/[C@@H](O)[C@H](CO)NC(=O)CCCCCCCCCCCCC] | 5 |
| So-44 | Cayman | 24357 | C8 Phytoceramide (t18:0/8:0) | CAS No 249728-93-6 N-Octanoyl Phytosphingosine | 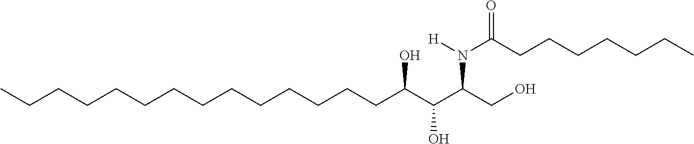 [SMILES: CCCCCCCCCCCCCC[C@@H](O)[C@@H](O)[C@H](CO)NC(=O)CCCCCCC] | 5 |
| So-45 | Cayman | 24367 | DL-erythro/threo Sphinganine (d18:0) | CAS No 3102-56-5 DL-erythro Sphinganine (d18:0) | 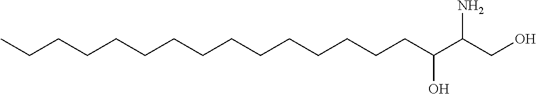 [SMILES: CCCCCCCCCCCCCCCC(O)C(N)CO] | 5 |
| So-46 | Cayman | 24374 | L-erythro Sphinganine (d18:0) | CAS No 6036-76-6 C18 L-erythro Sphinganine (d18:0) | 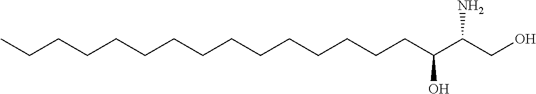 [SMILES: CCCCCCCCCCCCCCC[C@H](O)[C@H](N)CO] | 5 |
| So-47 | Cayman | 24372 | L-erythro Sphingosine (d18:1) | CAS No 6036-75-5 L-erythro-C18-Sphingosine | 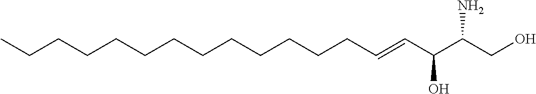 [SMILES: CCCCCCCCCCCCC/C=C/[C@H](O)[C@H](N)CO] | 5 |
| So-48 | Cayman | 24385 | N,N-dihexyl Sphingosine (d18:1/6:0/6:0) | N,N-dihexyl-D-erythro- Sphingosine | 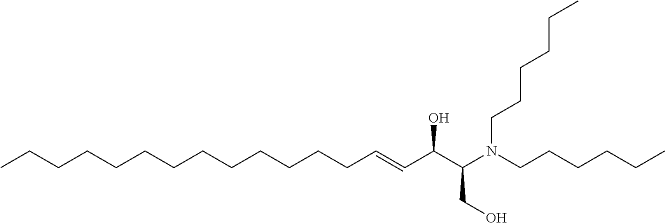 [SMILES: CCCCCCCCCCCCC/C=C/[C@@H](O)[C@H](CO)N(CCCCCC)CCCCCC] | 5 |
| So-49 | Cayman | 23211 | 1-β-D- Glucosylsphingosine (d18:1) | CAS No 52050-17-6 1-β-D-Glucosylsphingosine (synthetic) | 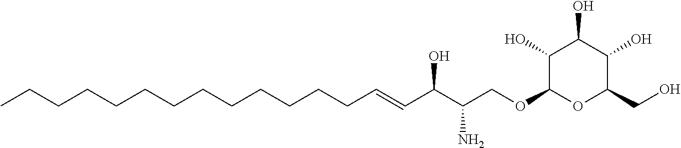 [SMILES: CCCCCCCCCCCCC/C=C/[C@@H](O)[C@@H](N)CO[C@@H]1O[C@H](CO)[C@@H](O)[C@H](O)[C@H]1O] | 5 |
| So-50 | Cayman | 24435 | C12((±)-2′-hydroxy) dihydro Ceramide (d18:0/12:0) | N-(R,S)-α- hydroxydodecanoyl-D- erythro- Dihydrosphingosine | 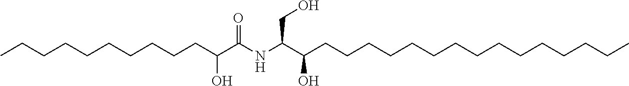 [SMILES: CCCCCCCCCCCCCCC[C@@H](O)[C@H](CO)NC(=O)C(O)CCCCCCCCCC] | 5 |
| So-51 | Cayman | 22823 | C2 Phytoceramide (t18:0/2:0) | Ceramide (t18:0/2:0) | 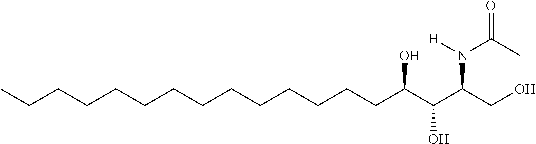 [SMILES: CCCCCCCCCCCCCC[C@@H](O)[C@@H](O)[C@H](CO)NC(C)=O] | 5 |
| So-52 | Cayman | 19579 | Ganglioside GM1 Mixture | CAS No 37758-47-7 Monosialoganglioside GM1 Mixture | 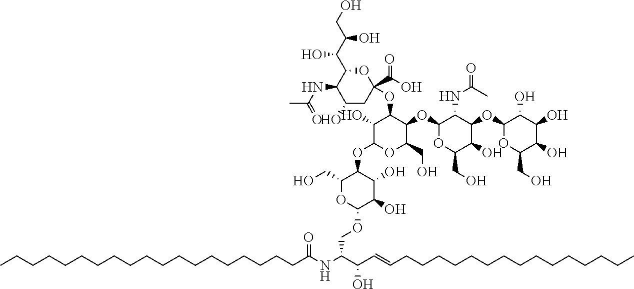 [SMILES: CCCCCCCCCCCCCCC/C=C/[C@H](O)[C@@H](CO[C@@H]1O[C@H](CO)[C@@H](OC2O[C@H](CO)[C@H](O[C@@H]3O[C@H](CO)[C@H](O)[C@H](O[C@@H]4O[C@H](CO)[C@H](O)[C@H](O)[C@H]4O)[C@H]3NC(C)=O)[C@H](O[C@]3(C(=O)O)C[C@H](O)[C@@H](NC(C)=O)[C@H]([C@H](O)[C@H](O)CO)O3)[C@H]2O)[C@H](O)[C@H]1O)NC(=O)CCCCCCCCCCCCCCCCCCC] | 5 |
| So-53 | Cayman | 10007902 | Sphingosine (d17:1) | CAS No 6918-48-5 D-erythro-Sphingosine C- 17 | 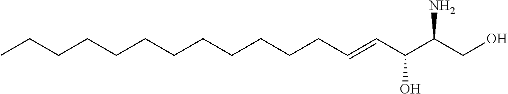 [SMILES: CCCCCCCCCCCC/C=C/[C@@H](O)[C@@H](N)CO] | 5 |
| So-54 | Cayman | 62525 | C6 Ceramide (d18:1/6:0) | CAS No 124753-97-5 N-hexanoyl-D-erythro- sphingosine | 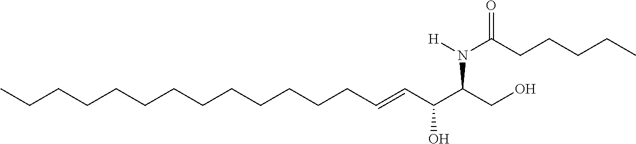 [SMILES: CCCCCCCCCCCCC/C=C/[C@@H](O)[C@H](CO)NC(=O)CCCCC] | 5 |
| So-55 | Cayman | 24375 | D-threo Sphinganine (d18:0) | CAS No 6036-86-8 D-threo- Dihydrosphingosine | 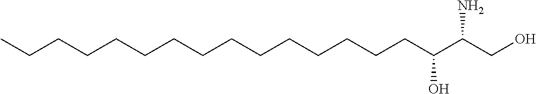 [SMILES: CCCCCCCCCCCCCCC[C@@H](O)[C@H](N)CO] | 5 |
| So-56 | Cayman | 62530 | C24:1 Ceramide (d18:1/24:1(15Z)) | CAS No 54164-50-0 N-Nervonoyl-D-erythro- Sphingosine | 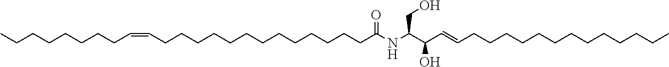 [SMILES: CCCCCCCC/C=C\CCCCCCCCCCCCCC(=O)N[C@@H](CO)[C@H](O)/C=C/CCCCCCCCCCCCC] | 5 |
| So-57 | Cayman | 24437 | C18 ((±)-2′-hydroxy) dihydro Ceramide (d18:0/18:0) | CAS No 215528-91-9 N-(2′-(R,S)- hydroxystearoyl)-D- erythro- Dihydrosphingosine | 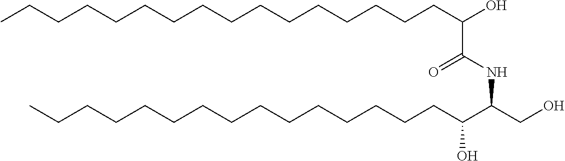 [SMILES: CCCCCCCCCCCCCCCCC(O)C(=O)N[C@@H](CO)[C@H](O)CCCCCCCCCCCCCCC] | 5 |
| So-58 | Cayman | 62540 | C8 Ceramide (d18:1.8:0) | CAS No 74713-59-0 N-octanoyl-D-erythro- Sphingosine | 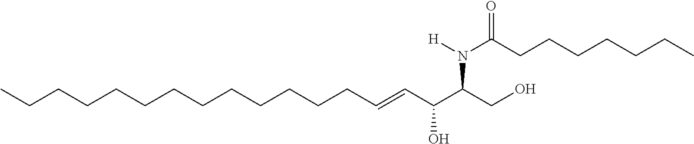 [SMILES: CCCCCCCCCCCCC/C=C/[C@@H](O)[C@H](CO)NC(=O)CCCCCCC] | 5 |
| So-59 | Cayman | 62510 | C2 Ceramide (d18:1/2:0) | CAS No 3102-57-6 N-acetoyl-D-erythro- sphingosine | 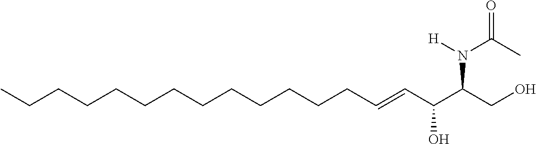 [SMILES: CCCCCCCCCCCCC/C=C/[C@@H](O)[C@H](CO)NC(C)=O] | 5 |
| So-60 | Larodan | 56-1095-4 | N-Acetyl- Sphingosyl- phosphoryl- ethanolamine CPEs | N-Acetyl- Sphingosylphosphoryl- ethanolamine | 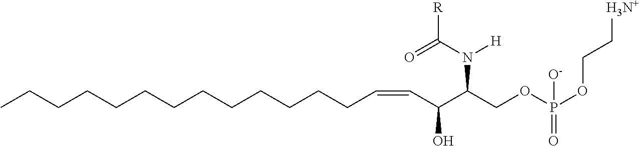 | 5 |
| So-61 | Larodan | 56-1096-4 | N-Pentadecanoyl- Psychosine | N-[(1S,2R,3E)-1-[(β-D- galactopyranosyl- oxy)methyl]-2- hydroxy-3-heptadecen- 1-yl]-pentadecanamide | 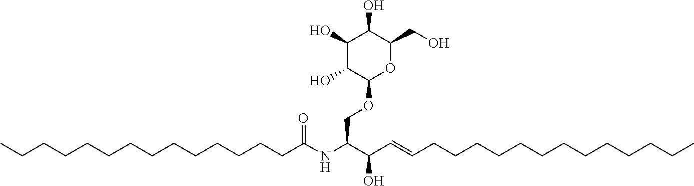 [SMILES: CCCCCCCCCCCCC/C=C/[C@@H](O)[C@H](CO[C@@H]1O[C@H](CO)[C@H](O)[C@H](O)[C@H]1O)NC(=O)CCCCCCCCCCCCCC] | 5 |
| So-62 | Larodan | 56-1120-5 | N-Lauroyl-D- Sphingosine | N-[(1S,2R,3E)-2-hydroxy- 1-(hydroxymethyl)-3- heptadecen-1-yl]- dodecanamide | 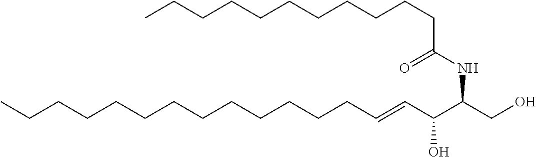 [SMILES: CCCCCCCCCCCCC/C=C/[C@@H](O)[C@H](CO)NC(=O)CCCCCCCCCCC] | 5 |
| So-63 | Larodan | 56-1125-5 | Phytosphingosine | 2S-amino-1,3S,4R- octadecanetriol | 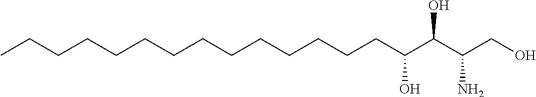 [SMILES: CCCCCCCCCCCCCC[C@@H](O)[C@@H](O)[C@@H](N)CO] | 5 |
| So-64 | Larodan | 56-1135-5 | N-Pentadecanoyl-D- Sphingosine | N-[(1S,2R,3E)-2-hydroxy- 1-(hydroxymethyl)-3- heptadecen-1-yl]- pentadecanamide | 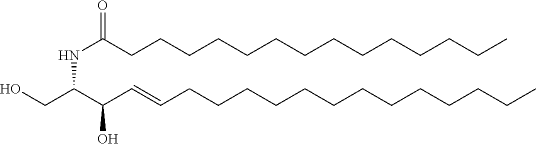 [SMILES: CCCCCCCCCCCCC/C=C/[C@@H](O)[C@H](CO)NC(=O)CCCCCCCCCCCCCC] | 5 |
| So-65 | Larodan | 56-1137-5 | N-Nonadecanoyl-D- Sphingosine | N-[(1S,2R,3E)-2-hydroxy- 1-(hydroxymethyl)-3- heptadecen-1-yl]- nonadecanamide | 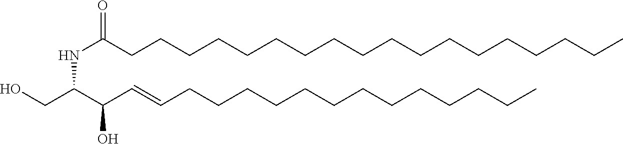 [SMILES: CCCCCCCCCCCCC/C=C/[C@@H](O)[C@H](CO)NC(=O)CCCCCCCCCCCCCCCCCC] | 5 |
| So-66 | Larodan | 56-1141-4 | Glucosyl- sphingosine(d18:1) | D-glucosyl-β1-1′-D- erythro-sphingosine, | 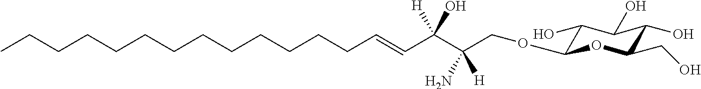 | 5 |
| So-67 | Larodan | 56-1161-4 | N-Hexanoyl- Phytosphingosine, Cer(t18:0/6:0) | N-[(1S,2S,3R)-2,3- dihydroxy-1- (hydroxymethyl)hepta- decyl]-hexanamide | 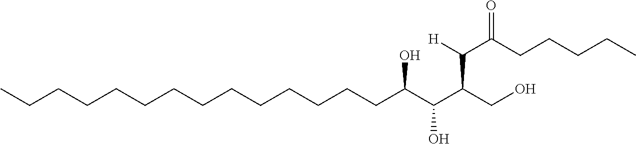 [SMILES: CCCCCCCCCCCCCC[C@@H](O)[C@@H](O)[C@H](CO)CC(=O)CCCCC] | 5 |
| So-68 | Larodan | 56-1163-4 | N-Palmitoyl- Phytosphingosine, Cer(t18:0/16:0) | N-[(1S,2S,3R)-2,3- dihydroxy-1-(hydroxy- methyl)heptadecyl]- hexadecanamide | 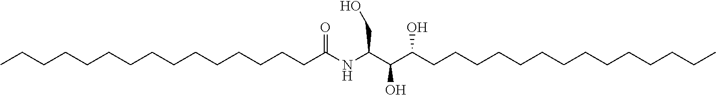 [SMILES: CCCCCCCCCCCCCCCC(=O)N[C@@H](CO)[C@H](O)[C@H](O)CCCCCCCCCCCCCC] | 5 |
| So-69 | Larodan | 56-1164-4 | N-Stearoyl- Phytosphingosine, Cer(t18:0/18:0) | N-[(1S,2S,3R)-2,3- dihydroxy-1-(hydroxy- methyl)heptadecyl]- octadecanamide | 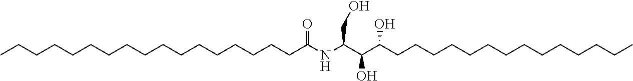 [SMILES: CCCCCCCCCCCCCCCCCC(=O)N[C@@H](CO)[C@H](O)[C@H](O)CCCCCCCCCCCCCC] | 5 |
| So-70 | Larodan | 56-1304-4 | trans L-erythro- Sphingosine (synthetic) | (2R,3S,4E)-2-amino-4- octadecene-1,3-diol | 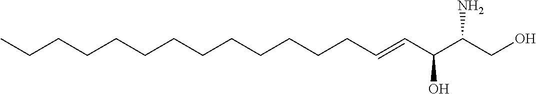 [SMILES: CCCCCCCCCCCCC/C=C/[C@H](O)[C@H](N)CO] | 5 |
| So-71 | Larodan | 56-1306-5 | DL- erythro/threo Sphinganine (d18:0) | (2S,3R)-2-amino-1,3- Octadecanediol | 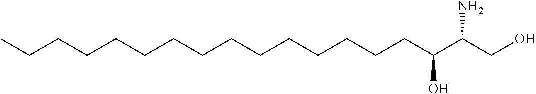 [SMILES: CCCCCCCCCCCCCCC[C@H](O)[C@H](N)CO] | 5 |
| So-72 | Larodan | 56-1315-5 | 3-keto Sphinganine (d12:0) | 2-amino-1-hydroxy-3- dodecanone, monohydrochloride | 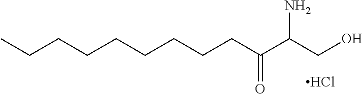 | 5 |
| So-73 | Larodan | 56-1326-4 | C15-D-erythro- Sphingosine | 2S-amino-4E-pentadecene- 1,3R-diol | 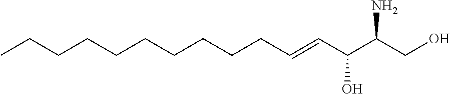 [SMILES: CCCCCCCCCC/C=C/[C@@H](O)[C@@H](N)CO] | 5 |
| So-74 | Larodan | 71-1302-1 | D-erythro- Sphingosine, D9 | (2S,3R,E)-aminooctadec- 4-ene- 15,15,16,16,17,17,18,18,18- d9-1,3-diol | 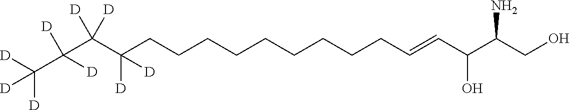 [SMILES: [2H]C([2H])([2H])C([2H])([2H])C([2H])([2H])C([2H])([2H])CCCCCCCCC/C=C/C(O)[C@@H](N)CO] | 5 |
| So-75 | Larodan | 56-1615-4 | 15-Methylhexadeca Sphinganine | 1,3-Hexadecanediol, 2- amino-15-methyl-, D- erythro- | 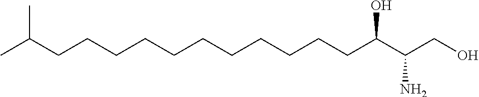 [SMILES: CC(C)CCCCCCCCCCC[C@@H](O)[C@@H](N)CO] | 5 |
| No. 41 | avanti | 792079P | Sphinganine(d22:0) | | 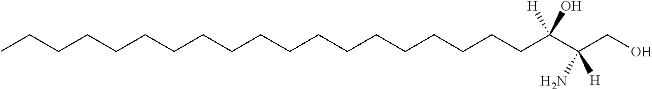 [SMILES: CCCCCCCCCCCCCCCCCCC[C@@H](O)[C@@H](N)CO] | 10 |
| No. 38 | avanti | 791016 | PE(16:0/16:1) | (2-aminoethoxy)[(2R)-2- [(9Z)-hexadec-9- enoyloxy]-3- (hexadecanoyloxy)propoxy] phosphinic acid | 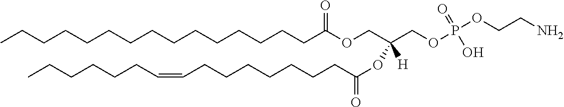 [SMILES: CCCCCC/C=C\CCCCCCCC(=O)O[C@H](COC(=O)CCCCCCCCCCCCCCC)COP(=O)(O)OCCN] | 10 |
| No. 48 | Larodan | 34-2230 | TG(18:1/22:1/22:1) | 1,2-Eucin(13Z)-3-Olein | 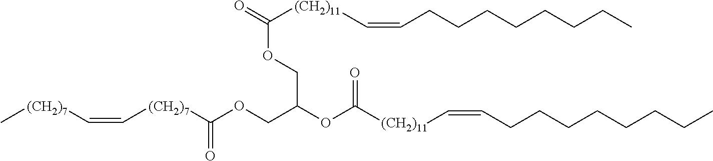 | 10 |

TABLE 2
Synthesis information of small RNA (purchased
from Guangzhou RiboBio Co., LTD, Ribobio)

| | Single-/ | | |
|---|---|---|---|
| | double- | | |
| Title | stranded | Sequence | Concentration |
| PGY-ssRNA-6 | single- | GTTCAGAGTTCTACA | 20 μM |
| | stranded | GTCCGA SEQ ID | |
| | | NO: 1) | |

TABLE 3
PBS formulation having a total system of 2 Liters
(reagents purchased from Beijing Chemical Works)

| PBS total system | 2 liters |
|---|---|
| NaCl | 16 g |
| Na2HPO4•12H2O | 7.16 g |
| KH2PO4 | 0.48 g |
| KCl | 0.4 g |

### EXAMPLES

### Experimental Methods

### 1. Cell Culture

The human embryonic kidney cell line HEK293T and human monocyte THP-1 used in the experiment were purchased from the Cell Culture Center of Peking Union Medical College. The cells were cultured in a 37° C., 5% CO₂ incubator, wherein HEK293T cells were cultured in DMEM medium (HyClone) and THP-1 cells were cultured in RPMI-1640 medium (HyClone), each medium containing 10% fetal bovine serum and a certain proportion of antibiotics (penicillin 100 U/ml & streptomycin 100 mg/ml). The cells were cultured to logarithmic growth phase, and the cell density was 6×10⁵/1 mL medium/well. The 12-well plate (1 mL medium/well) was incubated overnight (12 h) at 37° C., followed by subsequent experiments.

### 2. Preparation of System of Sphingosine Lipid for Delivering Small RNA

2.1 5 μl of small RNA (Ribobio, 20 μM, as shown in Table 2) and 95 μL of DEPC (Sigma) treated water were added into a 1.5 ml EP tube and mixed well. Then a certain amount of single lipid or lipid composition in chloroform solution (as shown in Table 1) was added and fully mixed by sucking and blowing;
2.2 The system was fully mixed and was heated in a water bath at 90° C. for 15 minutes;
2.3 The system was taken out and cooled down to room temperature.

### 3. Real-Time Quantitative PCR (RT-qPCR) Detection of Intracellular Expression of Nucleic Acids Delivered by Lipid

3.1 The human embryonic kidney cell line HEK293T used in the experiment was cultured to logarithmic growth phase, and then plated on a 12-well plate with a cell density of 6×10³/1 mL medium/well. The 12-well plate (1 mL medium/well) was incubated overnight (12 h) at 37° C., followed by subsequent experiments.
3.2 Experimental groups were as follows:
1) Naïve group: untreated cells. This group served as a blank control group;
2) Free uptake group: small RNA solution was directly added (the storage concentration was 20 μM). This group served as a negative control group;
3) Lipid nucleic acid mixture: the mixture of lipid and small RNA prepared from the step 2 was added into cells and mixed, and the final concentration of small RNA was 100 nM.
3.3 After co-incubation with cells for a specific period of time, the cells were washed three times with PBS. The cells were harvested with TRIzol lysis buffer (purchased from Sigma-Aldrich), and total RNA was extracted. The abundance of small RNA that entered the cells was detected by RT-qPCR; the protocols were as follows:
1) Extraction of total cellular RNA:

To the cells cultured in a 12-well plate (about 1×10⁶cells/well) was added 1 mL TRIzol lysis buffer in each well, and then placed on ice. After all the samples were added TRIzol, they were allowed to stand at room temperature for 5 min to be fully lysed.

Centrifugation was conducted at 4° C., 12,000 rpm for 5 min. The precipitates were discarded and TRIzol was transferred to a fresh centrifuge tube;

Chloroform was added at a ratio of 200 μL chloroform/mL TRIzol, shaken well and mixed. The mixture was placed at room temperature for 5 min;

Centrifugation was conducted at 4° C., 12,000 rpm for 15 min;

The upper aqueous phase was pipetted into another centrifuge tube, and isopropanol was added at a ratio of 0.5 mL isopropanol/mL TRIzol. The mixture was placed at room temperature for 5-10 min;

Centrifugation was conducted at 4° C., 12,000 rpm for 15 min. The supernatant was discarded, and the RNA precipitates to the bottom of the tube;

The tube was added 1 mL 75% ethanol and gently shaken to suspend the precipitates;

Centrifugation was conducted at 4° C., 12,000 rpm for 10 min. The supernatant was discarded. The tube was added 1 mL 75% ethanol acid gently shaken to suspend the precipitate;

Centrifugation was conducted at 4° C., 12,000 rpm for 10 min, and the supernatant was discarded. The DNA sample was dried at room temperature and dissolved with 50 μL RNase-free H₂O. The RNA concentration was quantified by measuring the OD value.
2) Total RNA was reverse transcribed to cDNA: Reverse Transcription Kit (High-Capacity cDNA Reverse Transcription Kits, Applied Biosystems, cat. no. 4368813) was used to reverse transcribe sRNA to cDNA by stem-loop method (see, e.g. Real-time quantification of microRNAs by stem-loop RT-PCR, Nucleic Acids Res. 2005 Nov. 27; 33(20):e179, incorporated by reference herein). The reverse transcription system was as follows: template RNA (150 ng/μL) 10 μL, 10× RT buffer 2.0 μL, 25× dNTP Mix (100 mM) 0.8 μL, U6 RT stem-loop primer 2.0 μL, HJT-sRNA-m7 RT stem-Loop primer 2.0 μL, MultiScribe™ reverse transcriptase 1.0 μL, RNase inhibitor 1.0 μL, nuclease-free H₂O 1.2 μL. The sample was loaded into a PCR reactor after short spin. The reaction conditions were as follows: (1) 25° C., 10 min; (2) 37° C., 120 min; (3) 85° C., 5 min; (4) 4° C., termination of reaction. 20 μl RNase-free ddH₂O was added to make up the final volume to 40 μl after the reaction. The stem-loop primer used in the reverse transcription process was synthesized by Beijing Tsingke Biotechnology Co., Ltd. (U6 RT primer, because the quantification of small RNA by RT-qPCR reaction can only be relative, so U6 was used as a standard reference gene for calculating the relative expression level): GTCGTATCCAGTGCAGGGTCCGAGGTATTCGCACTGGATACGACAAAAAT ATG (SEQ ID NO: 2); PGY-sRNA-m7 RT stem-loop primer: GTCGTATCCAGTGCACGCTCCGAGGTATTCGCACTGGATACGACTCGGAC (SEQ ID NO: 3)).
3) Quantitative PCR amplification reaction: the qPCR reaction system had a total volume of 10 μl, containing: 5 μL 2×SYBR Green Master Mix, 0.5 μl forward primer (10 μM), 0.5 μl reverse primer (10 μM), 1 μl cDNA obtained by reverse transcription, 3 μl RNase-free dH₂O. LightCycler 480 fluorescence quantitative PCR instrument was used, and the PCR reaction conditions were: 95° C., pre-denaturation for 5 min, followed by PCR amplification cycle: (1) 95° C., 10 s; (2) 55° C., 10 s; (3) 72° C., 20 s; a total of 40 cycles; 40° C. for 10 s in the end to cool down. Both the forward and reverse primers of the amplification reaction were designed and synthesized by Beijing Tsingke Biotechnology Co., Ltd. (U6 forward primer: GCGCGTCGTGAAGCGTTC (SEQ ID NO: 4), U6 reverse primer: GTGCAGGGTCCGAGGT (SEQ ID NO: 5), PGY-sRNA-m7 forward primer: TCGCGCGTTCAGAGTTC (SEQ ID NO: 6), PGY-sRNA-m7 reverse primer: GTGCACGCTCCGAGGT (SEQ ID NO: 7)).
4) 2-ΔCt method (relative gene expression level=2−(Ct target gene−Ct internal reference gene)) was used to calculate the relative amount of entry (single or double stranded RNA).
   4. Flow Cytometry Technology (CFlow) Determined the Uptake Amount of Nucleic Acid that is Delivered into Cells by Lipid
4.1 Main experimental instruments and equipment:
10 cm cell culture dishes, 12-well cell culture plates, pipettor, pipette, optical microscopes, flow cytometer Accuri® C6 instrument (purchased from BD, USA);
4.2 Main experimental reagents:

Model building and transfection: artificially synthesized single lipid shown in Table 1, artificially synthesized 5′-FAM labeled sRNAs (single-stranded, Ribobio) shown in Table 2;
4.3 The human embryonic kidney cell line HEK293T and human monocyte THP-1 cells used in the experiment were cultured to logarithmic growth phase, and then plated into 12-well plates with a cell density of 6×10⁵/1 mL medium/well. The 12-well plate (1 mL medium/well) was incubated overnight (12 h) at 37° C., followed by subsequent experiments.
4.4 The experiment groups were as follows:
1) Naïve group: untreated cells. This group served as a blank control group.
2) Free uptake group: 5 mL of 5′-FAM labeled small RNA solution (storage concentration is 20 μM) was directly added. This group served as a negative control group;
3) Lipid nucleic acid mixture treatment group: The mixture of the lipid prepared in step 2 and the 5′-FAM-labeled small RNA was added into the cells and mixed well. The final concentration of small RNA is 100 nM.
4.5 After co-incubation with cells for 6 h/9 h, the cells were washed three times with PBS. The cells were re-suspended with PBS (self-prepared). The flow cytometer Accuri® C6 instrument (purchased from BD, US) was used to detect the fluorescence intensity of the cells in the sample wells. Implementation method: the software CFlow Plus used in the flow cytometer Accuri® C6 instrument detection was opened, the instrument was washed with cleaning solution for 3 minutes, and then washed with double-distilled water for 5 minutes. The injection of blank group samples was started while setting the number of cells unlimited. The injection of Naive group samples was stopped when the number of cells detected was 10000 events. The live cell group in the fluorescence spectrum (abscissa: FSC-A, ordinate: SSC-A) was circled as gate P1. With the number of cells set as limited, the detection was stopped when the number of cells in gate P1 reached 10000 events. The fluorescence shift value was obtained in the fluorescence spectrum (abscissa: fluorescence channel FLA-1, ordinate: SSC-A).

### Example 1: Delivery of Small RNA into HEK293T Cell by Different Concentrations of Single Sphingosine Derivative

### (1) Experimental Groups:

A: Naïve group: untreated cells. This group served as a blank control group;
B: Free uptake group: the solvent CHCl₃ of sphingosine was used as a carrier to deliver FAM-labeled small RNA, and the final concentration of nucleic acid was 100 nM. This group served as a negative control group;
C: 0.1 nmol FAM-labeled single-stranded PGY-sRNA-6 was delivered into the cell by 3.75 μg of the single sphingosine derivative solution shown in Table 4, and the final concentration of nucleic acid was 100 nM;
D: 0.1 nmol FAM-labeled single-stranded PGY-sRNA-6 was delivered into the cell by 12.5 μg of the single sphingosine derivative solution shown in Table 4, and the final concentration of nucleic acid was 100 nM;
E: 0.1 nmol FAM-labeled single-stranded PGY-sRNA-6 was delivered into the cell by 37.5 μg of the single sphingosine derivative solution shown in Table 4, and the final concentration of nucleic acid was 100 nM;

### (2) Experimental Procedures

1) Conditions of the boiling method: to 100 μL FAM-labeled single-stranded PGY-sRNA-6 solution was added corresponding amount of lipid solution, and heated at 90° C. for 15 min;
2) Experimental conditions: the final concentration of small RNA was 100 nM. 9 hours after being added to the cells, the amount of single-stranded PGY-sRNA-6 that entered into the cells was compared by detecting the fluorescence shift using flow cytometry technology. For the detailed protocols, see “Flow cytometry technology (CFlow) determined the uptake amount of nucleic acid that is delivered into cells by lipid”. All experiments were performed in triplicates.

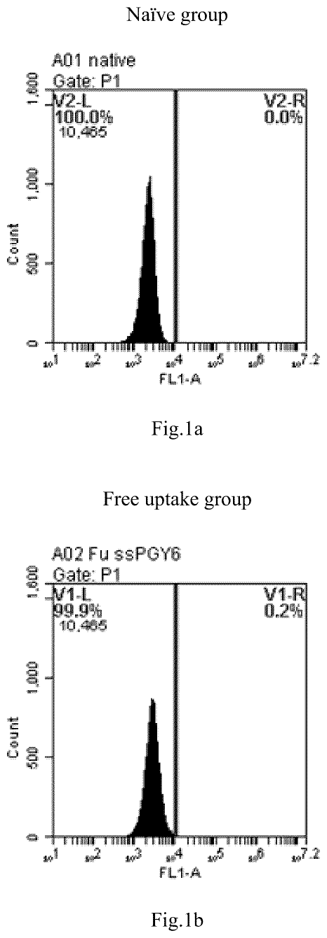

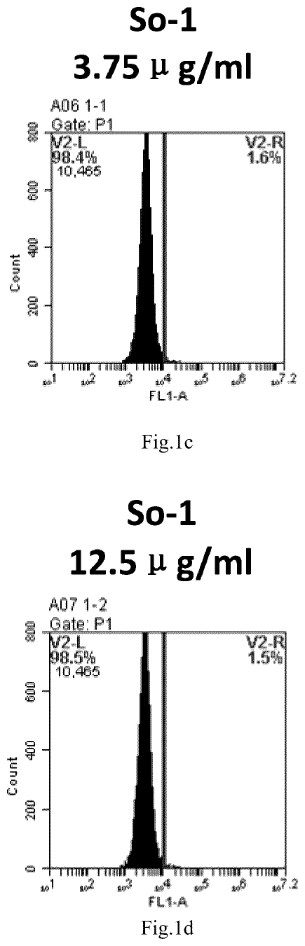

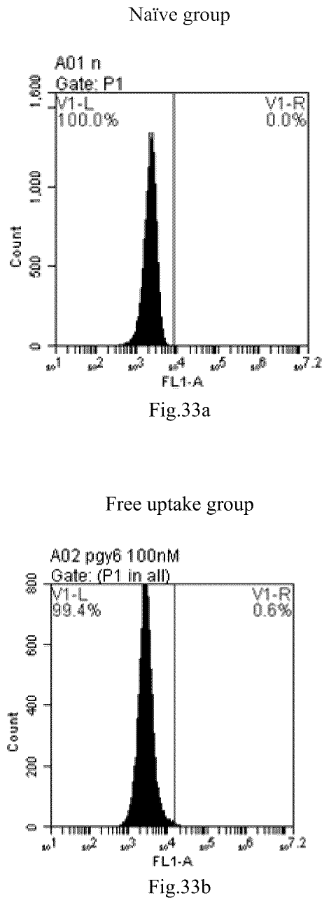

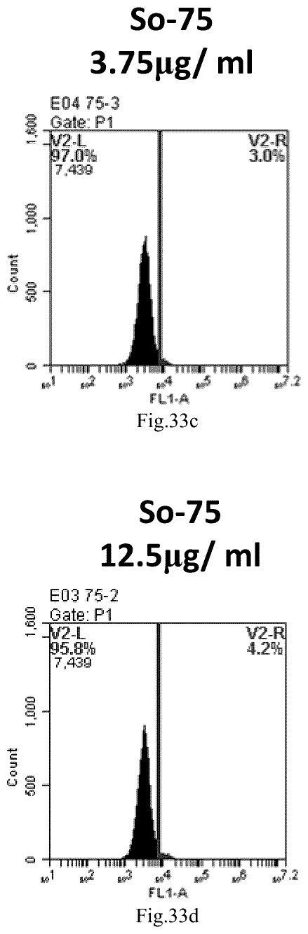

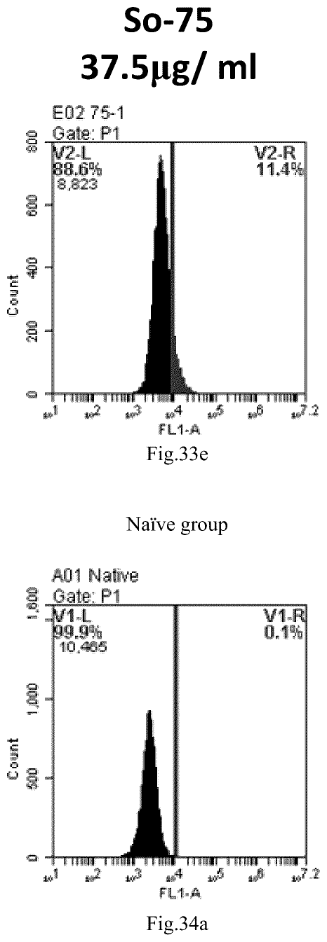

Conclusions: The results indicated that as compared to the free uptake group, the fluorescence value showed significant shift in the experimental group after delivery of fluorescently labeled nucleic acids by sphingosine lipids, indicating that different single sphingosine lipid derivatives were effective in delivering single-stranded small RNA into cells (see FIG. 1a-e to FIG. 33a-e). The effectiveness of single sphingosine lipid derivative for nucleic acid delivery, and the efficiency of sphingosine derivative to deliver sRNA were concentration dependent. Herein, the delivery by high-concentration sphingosine derivative (0.1 nmol small RNA was delivered into the cell by 37.5 μg of the single sphingosine derivative solution) had a more significant effect (see Table 4).

### Example 2: Delivery of Single-Stranded PGY-sRNA-6 into THP-1 Cell by Mixtures of Different Single sphingosine Derivatives, sphingosine (So) and phosphatidyl ethanolamine (PE)

### (1) Experimental Groups:

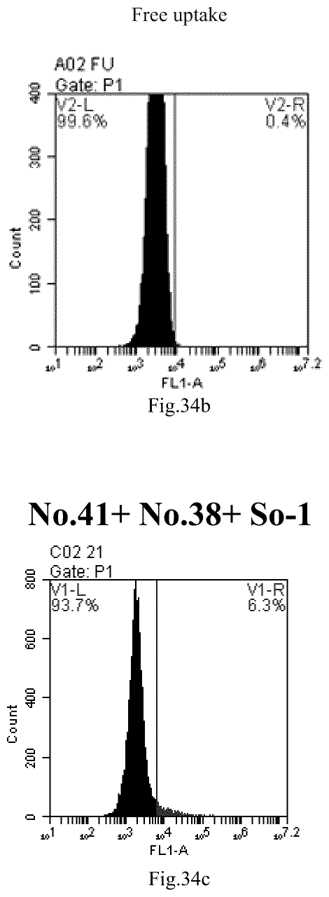

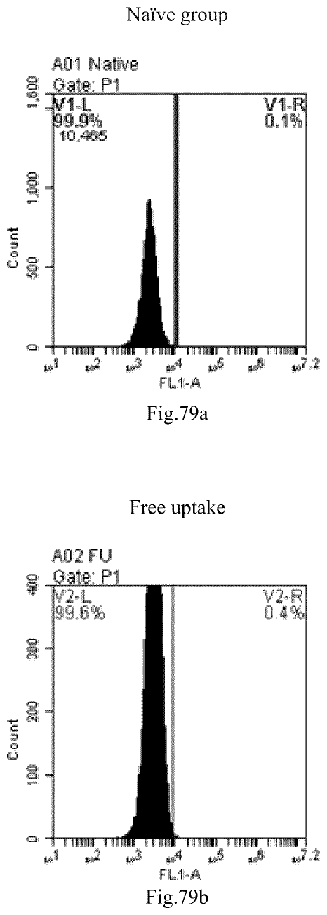

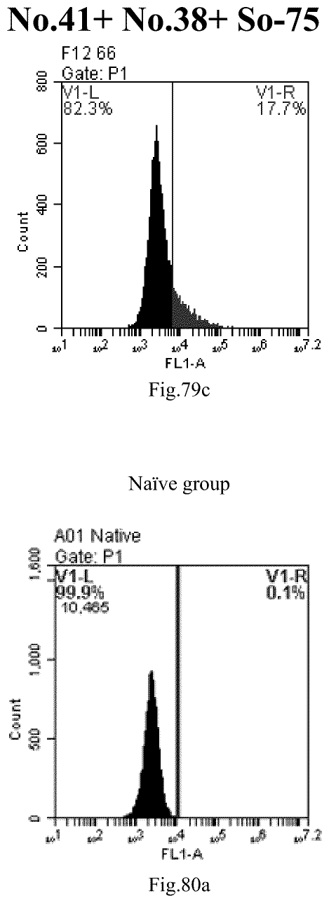

A: Naïve group: untreated cells. This group served as a blank control group;
B: Free uptake group: the solvent CHCl₃ of sphingosine was used as a carrier to deliver FAM-labeled small RNA, and the final concentration of nucleic acid was 100 nM. This group served as a negative control group;
C: Lipid mixture delivery group: 0.1 nmol FAM-labeled single-stranded PGY-sRNA-6 was delivered into the cell by 12.5 μg of the lipid mixture of sphingosine derivative (No. 41: No. 38: So=1:1:1, wherein the specific lipid mixture was shown in FIG. 34a-c to FIG. 79a-c and Table 4). The final concentration of nucleic acid was 100 nM.

### (2) Experimental Procedures

1) Conditions of the boiling method: to 100 μL FAM-labeled small RNA solution was added 12.5 μg of lipid mixture solution, and heated at 90° C. for 15 min;
2) Experimental conditions: the final concentration of small RNA was 100 nM. 6 hours after being added to the cells, the amount of small RNA that entered into the cells was compared by detecting the fluorescence shift using flow cytometry technology. For the detailed protocols, see “Flow cytometry technology (CFlow) determined the uptake amount of nucleic acid that is delivered into cells by lipid”. All experiments were performed in triplicates.

Conclusions: The results indicated that as compared to the free uptake group, the fluorescence value showed significant shift in the experimental group after delivery of nucleic acids by sphingosine lipids (see FIG. 34a-c to FIG. 79a-c. Table 4), indicating that 12.5 μg of lipid mixture was effective in delivering small RNA into cells. The effectiveness of lipid mixture (No. 41: No. 38: So=1:1:1) for nucleic acid delivery (see Table 4). Delivery of small RNA by lipid mixture had the potential of improving the efficiency of the delivery of nucleic acid drug in clinical settings.

### Example 3: Delivery of Single-Stranded PGY-sRNA-6 into THP-1 Cell by Mixtures of Different Single sphingosine Derivatives, sphingosine (So), phosphatidyl ethanolamine (PE) and triglyceride (TG)

### (1) Experimental Groups:

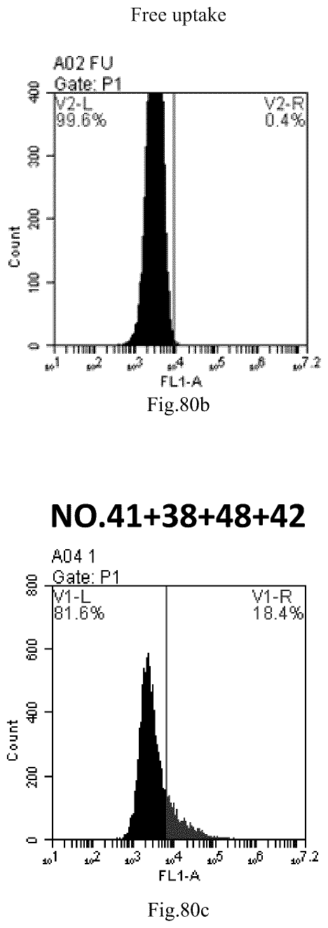

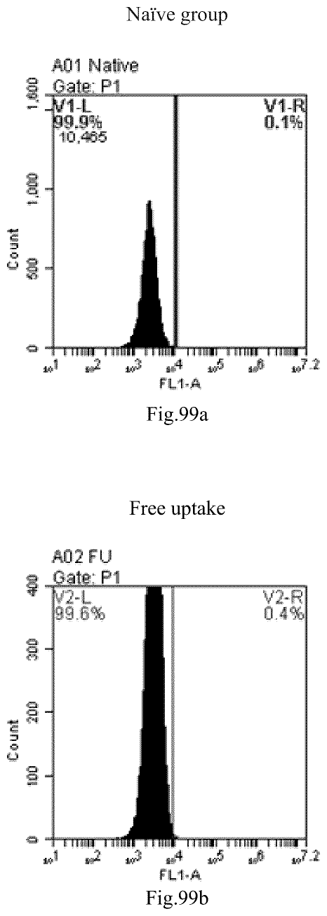

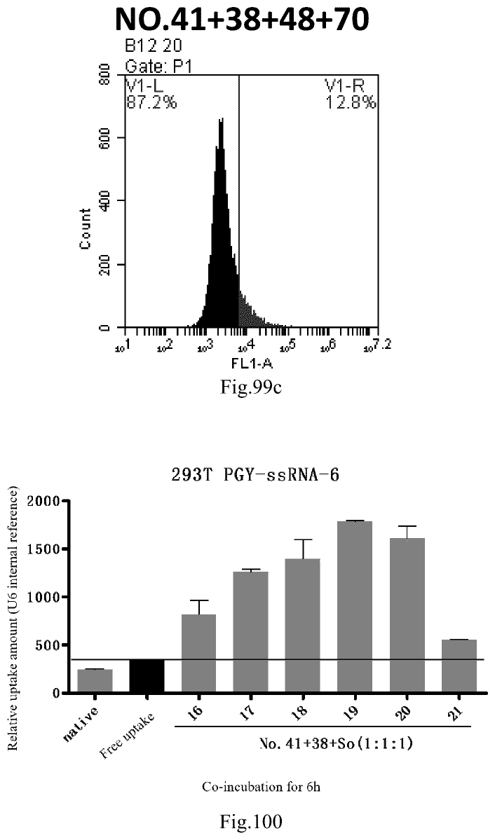

A: Naïve group: untreated cells. This group served as a blank control group;
B: Free uptake group: the solvent CHCl₃ of sphingosine was used as a carrier to deliver FAM-labeled small RNA, and the final concentration of nucleic acid was 100 nM. This group served as a negative control group;
C: Lipid mixture delivery group: 0.1 nmol FAM-labeled single-stranded PGY-sRNA-6 was delivered into the cell by 12.5 μg of the lipid mixture of sphingosine derivative (No. 41: No. 38: No. 48: So=2:2:1:2, wherein the specific lipid mixture was shown in FIG. 80a-c to FIG. 99a-c and Table 4), the final concentration of nucleic acid was 100 nM.

### (2) Experimental Procedures

1) Conditions of the boiling method: to 100 μL FAM-labeled small RNA solution was added 12.5 μg of lipid mixture solution, and heated at 90° C. for 15 min;
2) Experimental conditions: the final concentration of small RNA was 100 nM. 6 hours after being added to the cells, the amount of small RNA that entered into the cells was compared by detecting the fluorescence shift using flow cytometry technology. For the detailed protocols, see “Flow cytometry technology (CFlow) determined the uptake amount of nucleic acid that is delivered into cells by lipid”. All experiments were performed in triplicates.

Conclusions: The results indicated that as compared to the free uptake group, the fluorescence value showed significant shift in the experimental group after delivery of nucleic acids by sphingosine lipids (see FIG. 80a-c to FIG. 80a-c. Table 4), indicating that 12.5 μg of lipid mixture (No. 41: No. 38: No. 48: So=2:2:1:2) was effective in delivering small RNA into cells. The effectiveness of lipid mixture (No. 41: No. 38: No. 48: So=2:2:1:2) for nucleic acid delivery (see Table 4). Delivery of small RNA by lipid mixture had the potential of improving the efficiency of the delivery of nucleic acid drug in clinical settings.

### Example 4: Delivery of Single-Stranded PGY-sRNA-6 into HEK293T Cell by Mixtures of Different Single sphingosine Derivatives, sphingosine (So) and phosphatidyl ethanolamine (PE)

### (1) Experimental Groups:

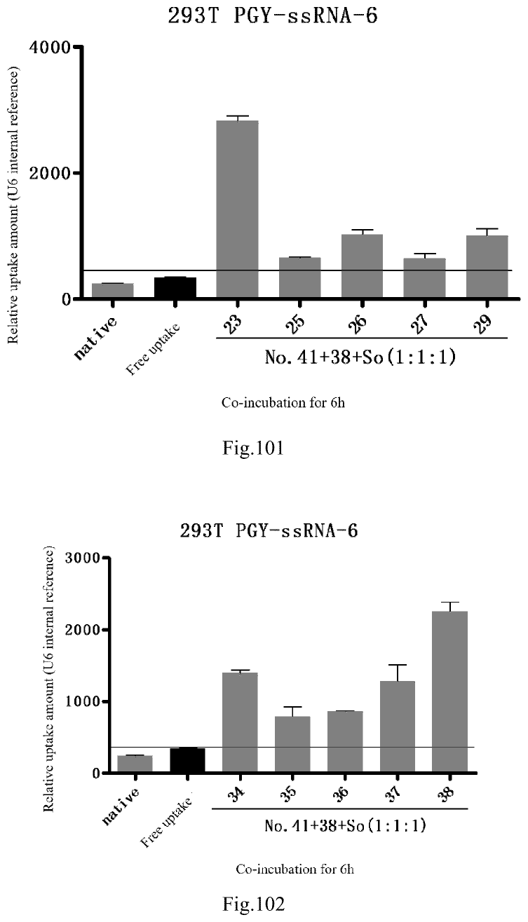

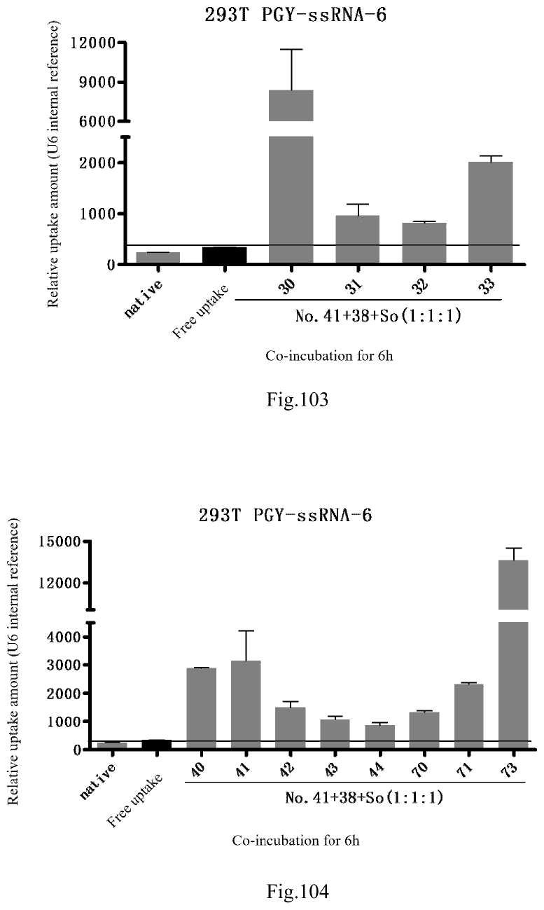

A: Naïve group: untreated cells. This group served as a blank control group;
B: Free uptake group: the solvent CHCl₃ of sphingosine was used as a carrier to deliver small RNA, and the final concentration of nucleic acid was 100 nM. This group served as a negative control group;
C: Lipid mixture delivery group: 0.1 nmol single-stranded PGY-sRNA-6 was delivered into the cell by 12.5 μg of the lipid mixture of sphingosine derivative (No. 41: No. 38: So=1:1:1, wherein the specific lipid mixture was shown in FIG. 100-104), the final concentration of nucleic acid was 100 nM.

### (2) Experimental Procedures

1) Conditions of the boiling method: to 100 μL small RNA solution was added corresponding amount of lipid solution, and heated at 90° C. for 15 min;
2) Experimental conditions: the final concentration of small RNA was 100 nM. 6 hours after being added to the cells, the amount of small RNA that entered into the cells was compared by RT-qPCR detection. For the detailed protocols, see “Real-time quantitative PCR (RT-qPCR) detection of intracellular expression of nucleic acids delivered by lipid”. All experiments were performed in triplicates.

Conclusions: The results indicated that as compared to the Naïve group and the negative control group, a significant increase in relative uptake amount of small RNA in cells was detected after delivery of 0.1 nmol nucleic acids by 12.5 μg of the lipid combination (No. 41: No. 38: So=1:1:1) containing sphingosine derivative in the experimental group, indicating that the addition of multiple single sphingosine lipid derivatives could greatly improve the efficiency of the delivery of lipid combinations.

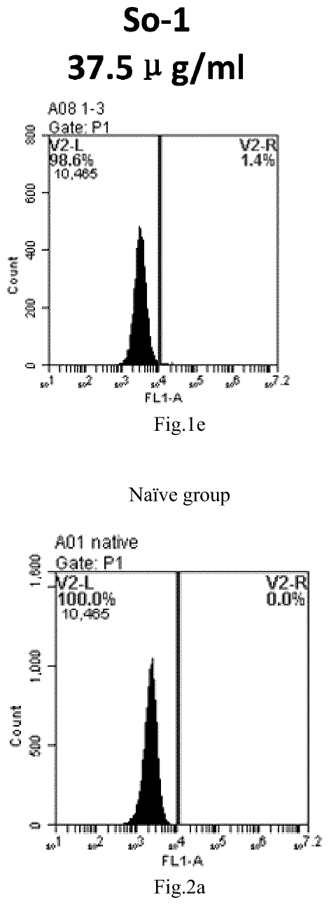

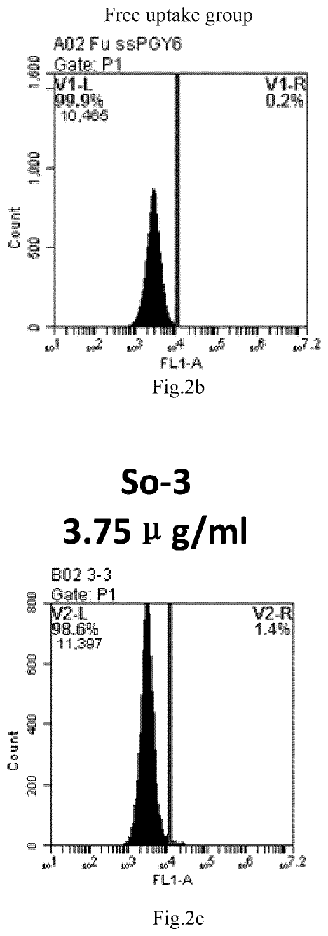

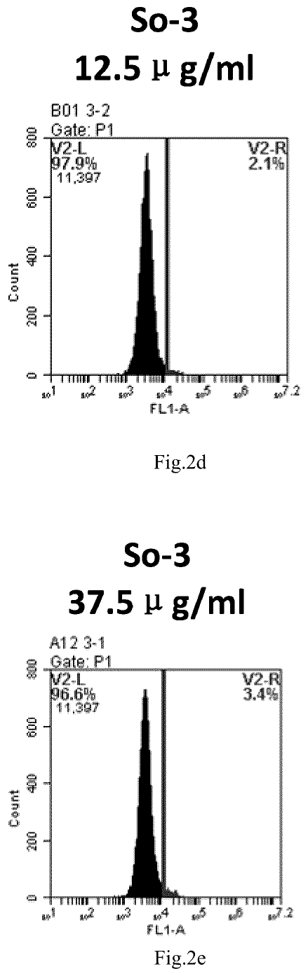

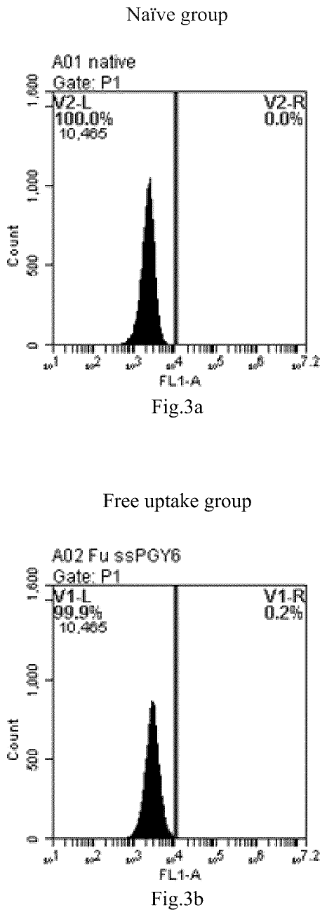

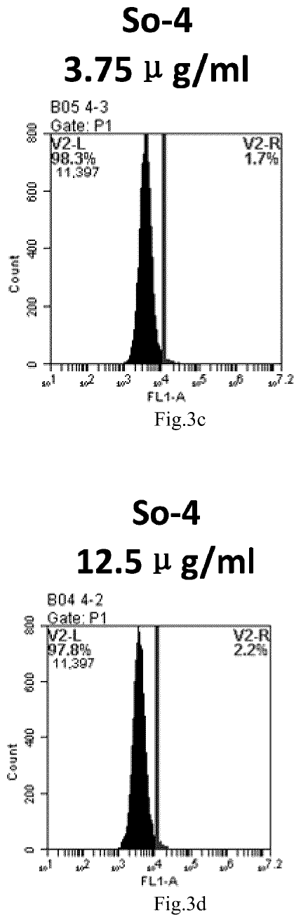

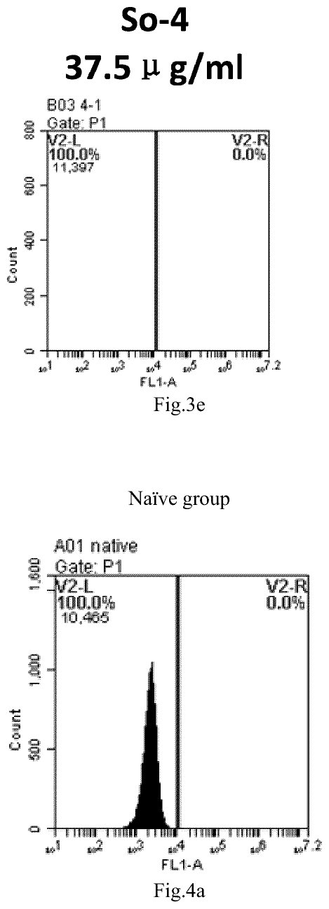

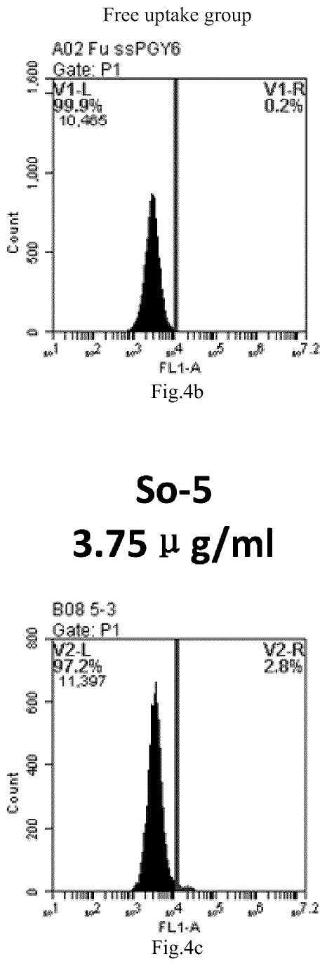

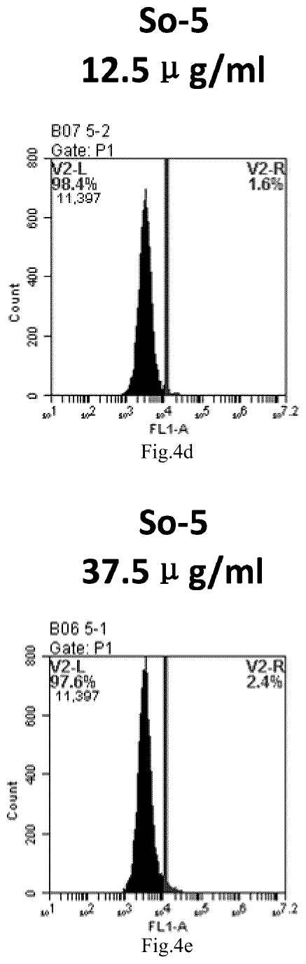

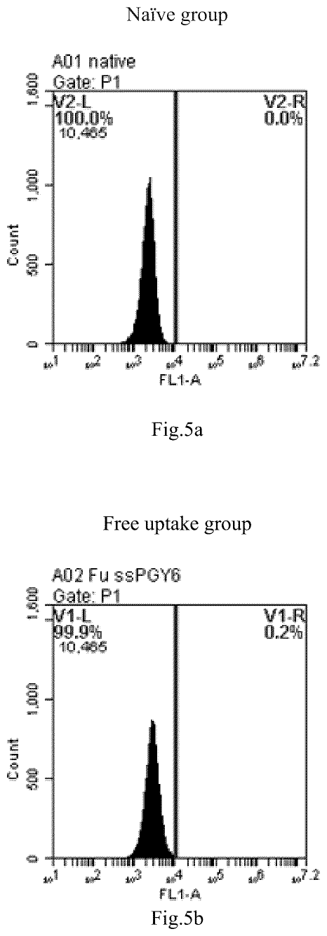

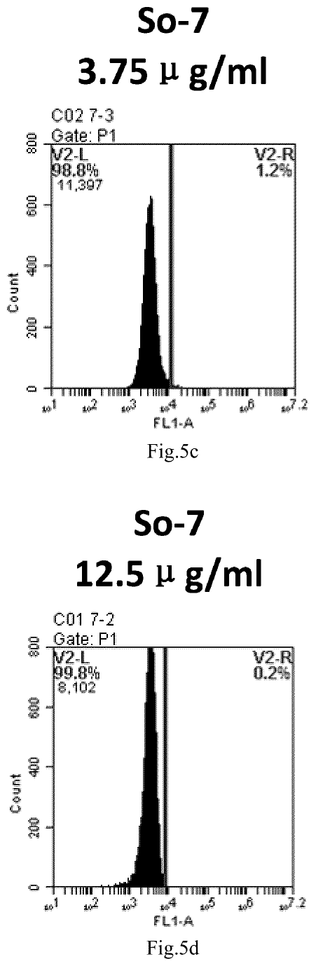

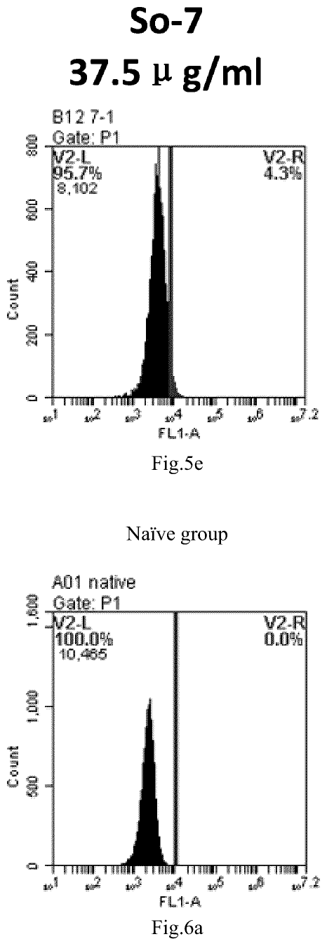

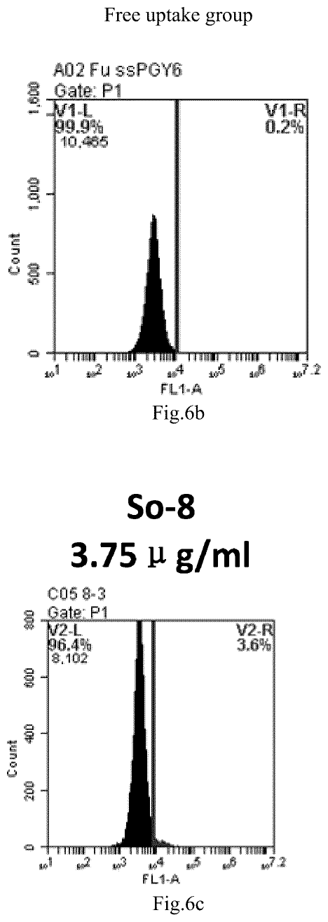

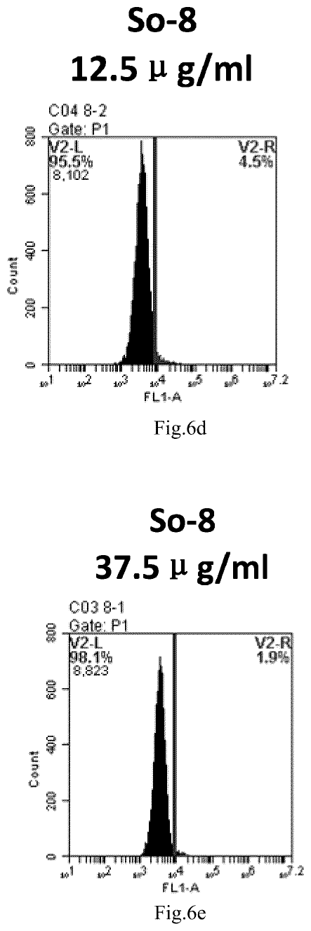

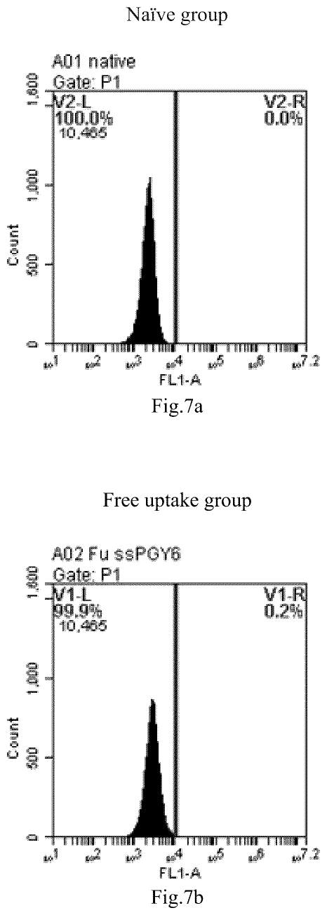

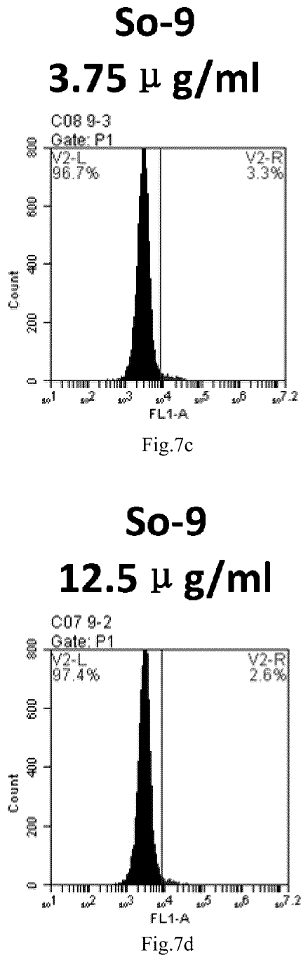

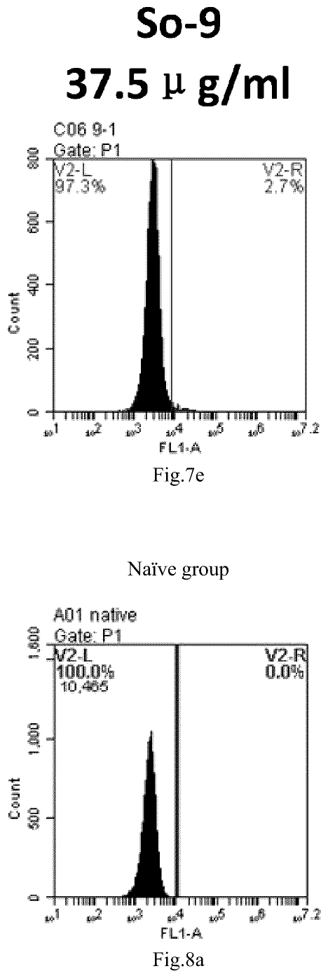

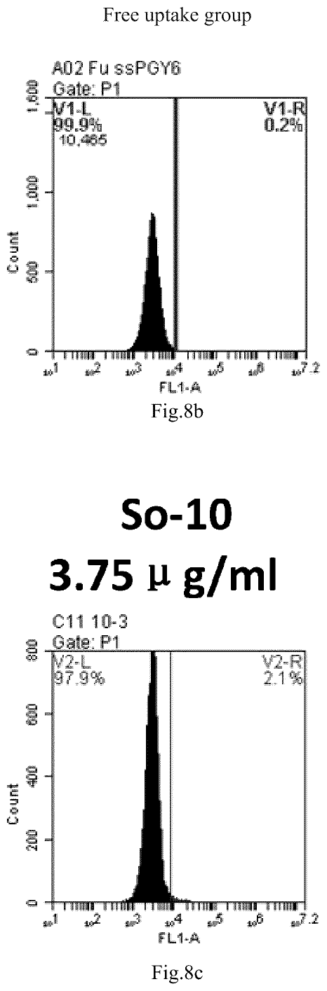

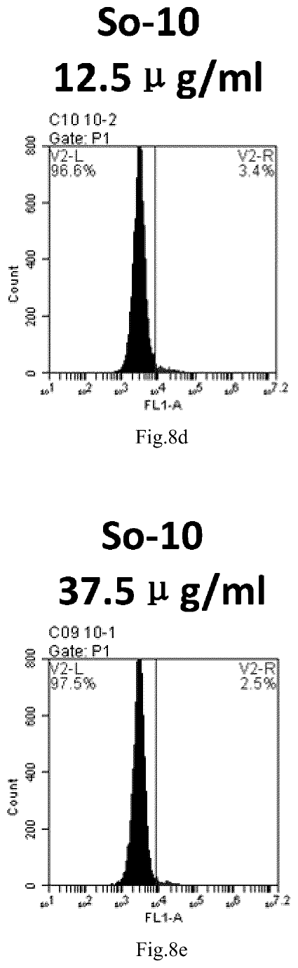

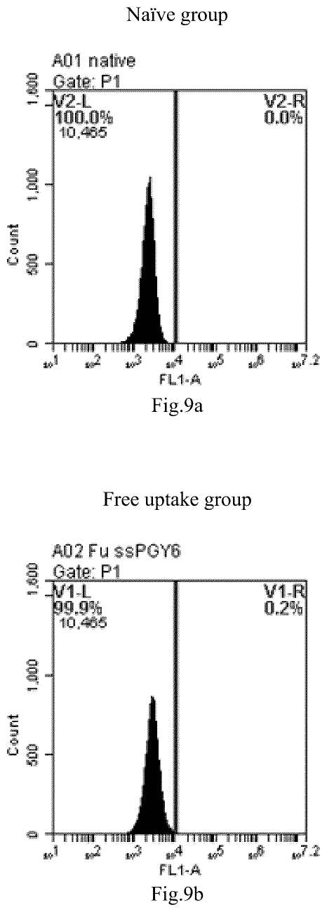

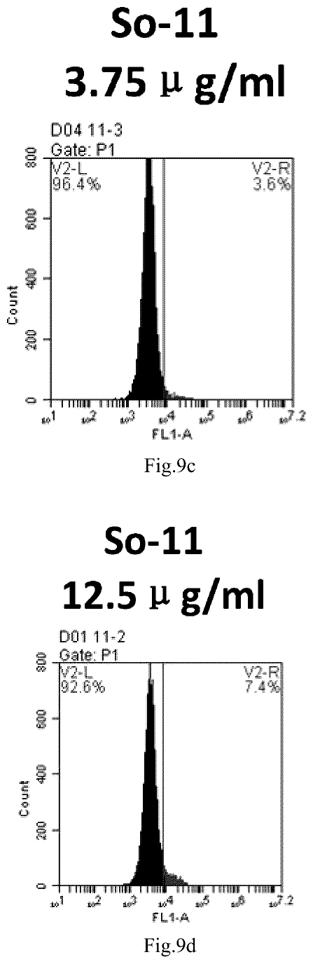

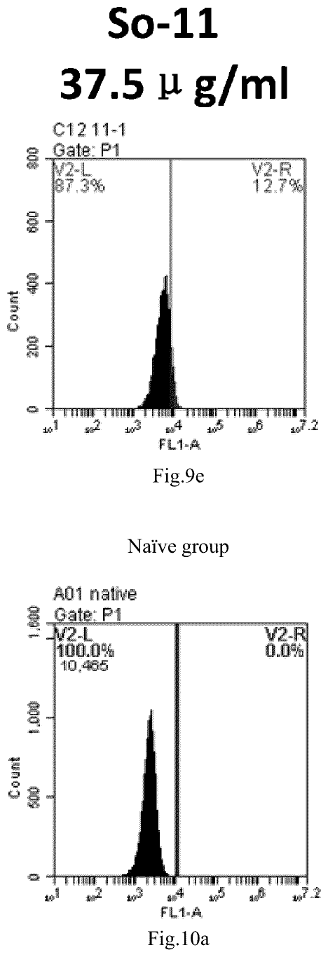

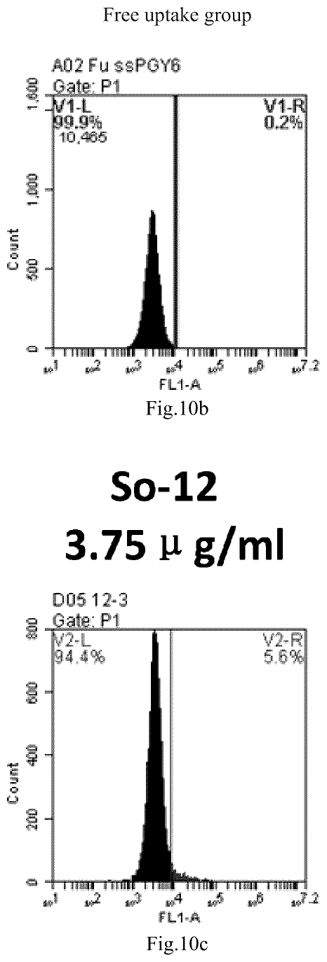

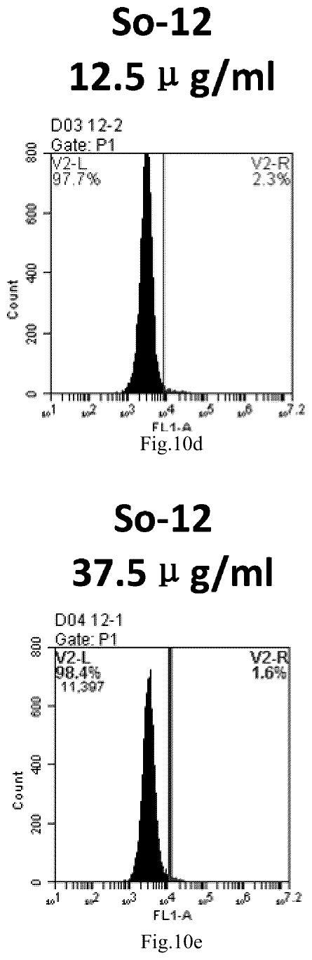

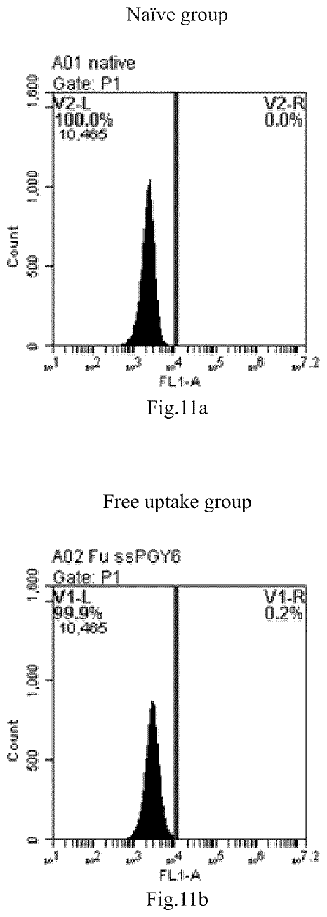

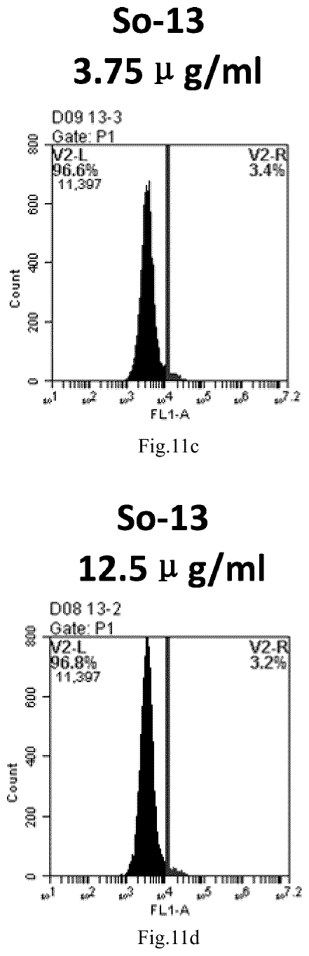

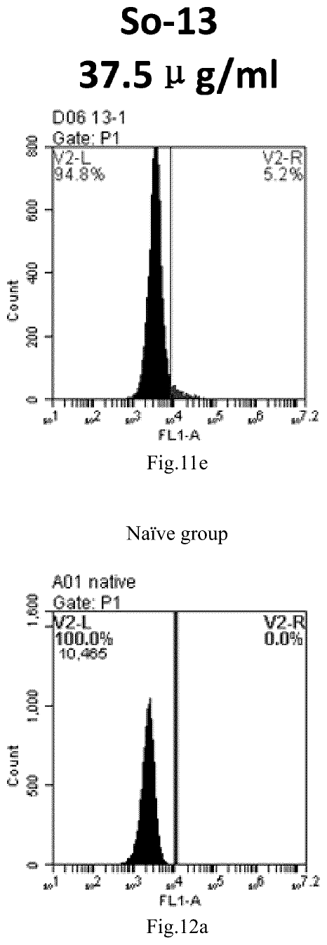

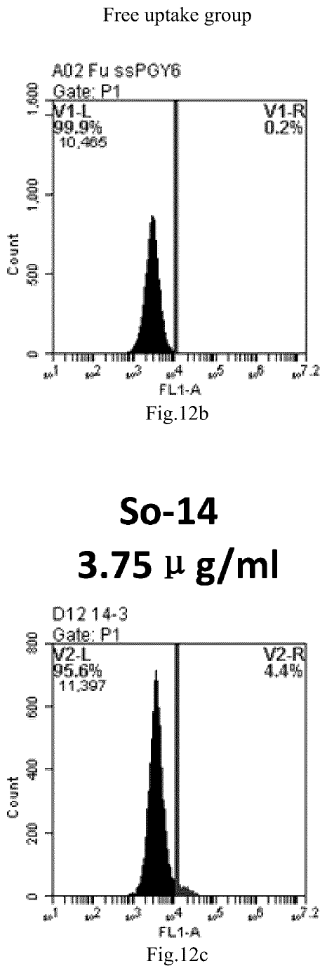

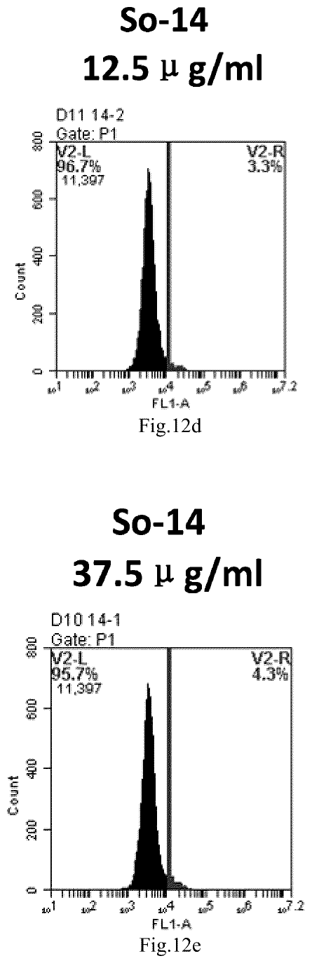

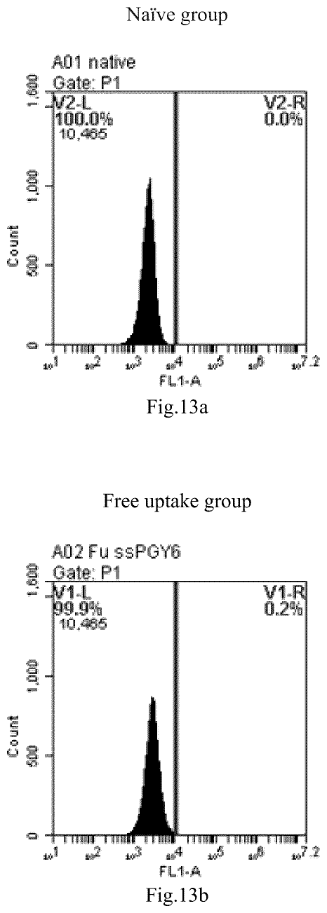

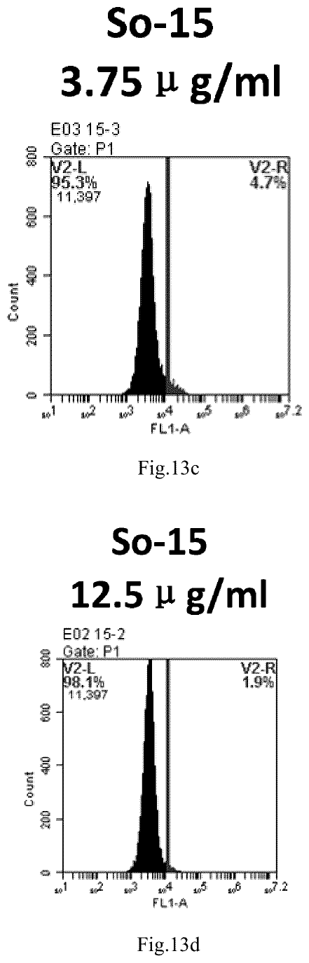

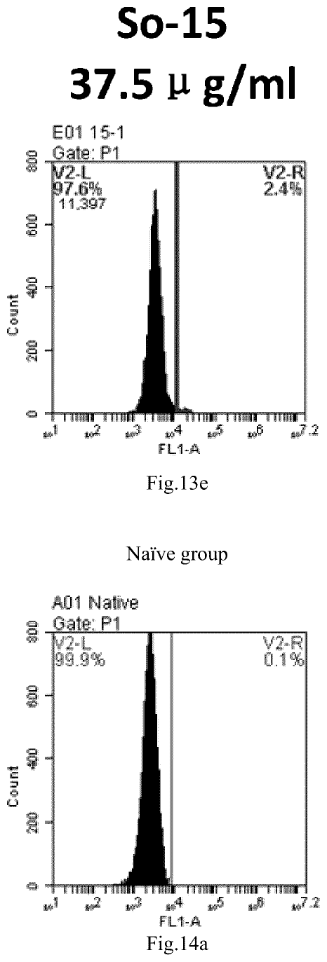

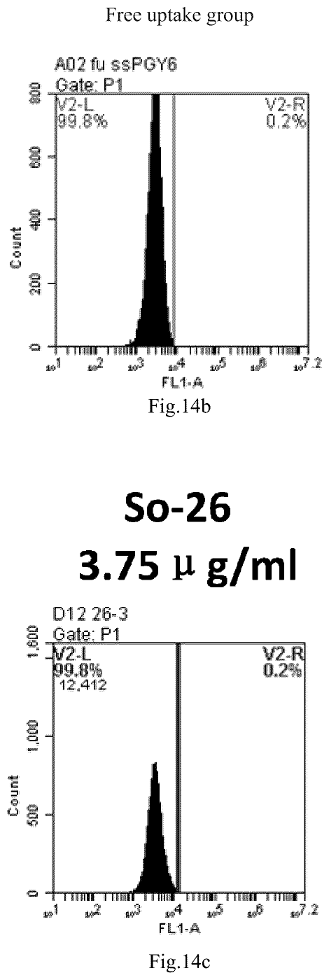

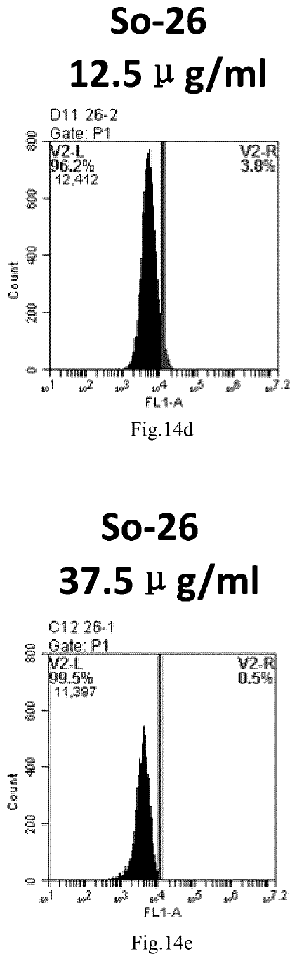

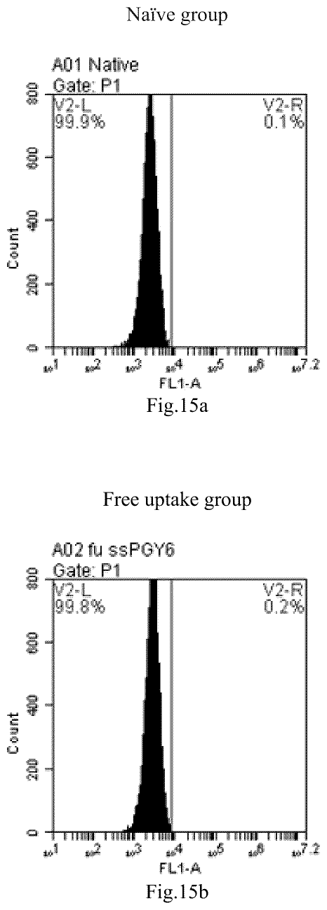

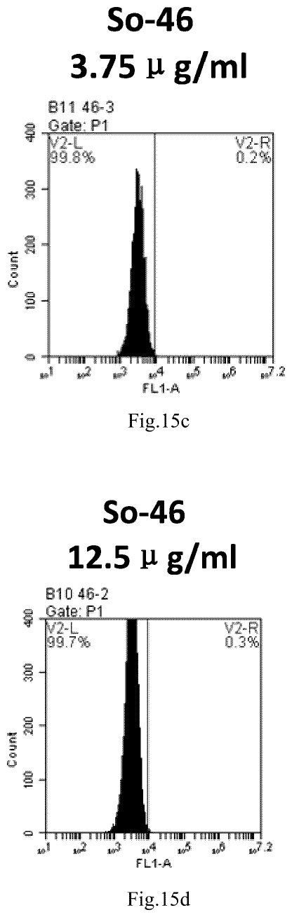

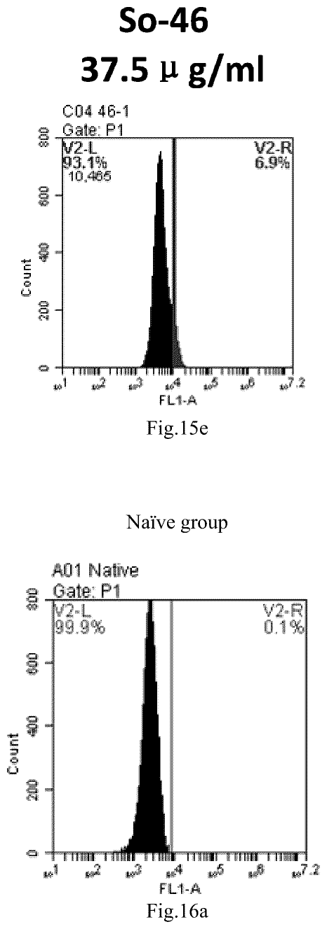

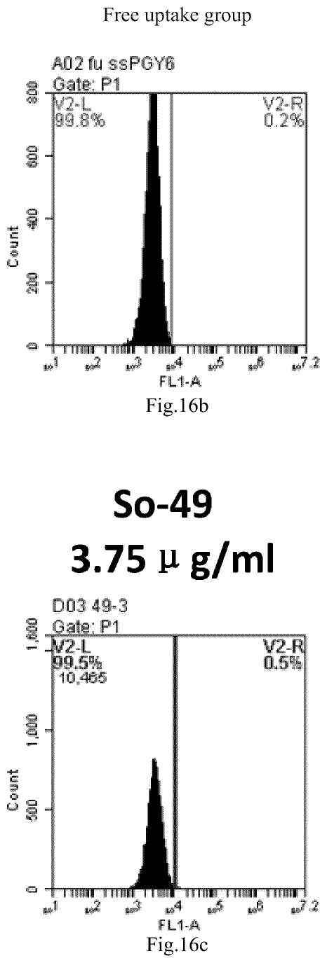

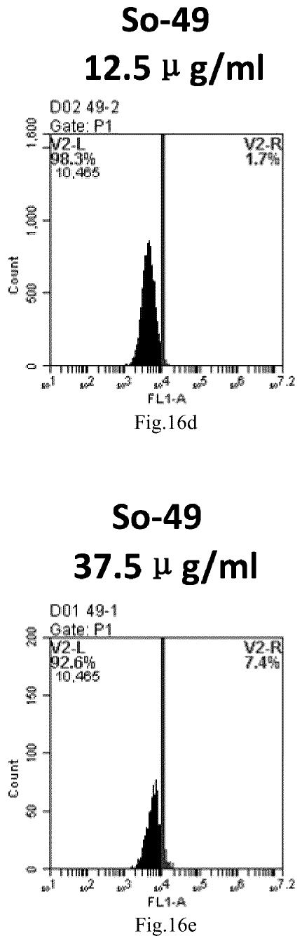

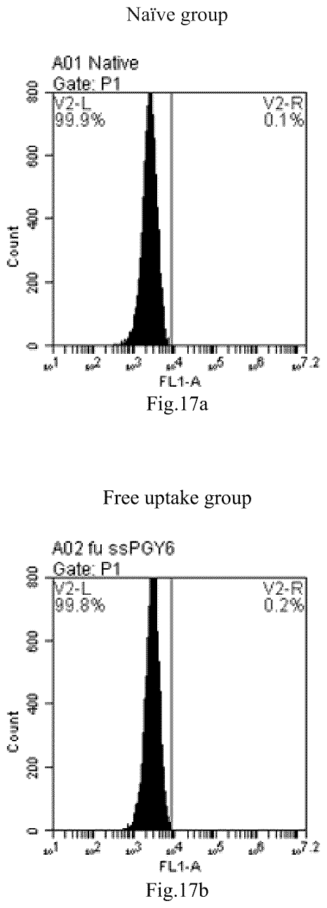

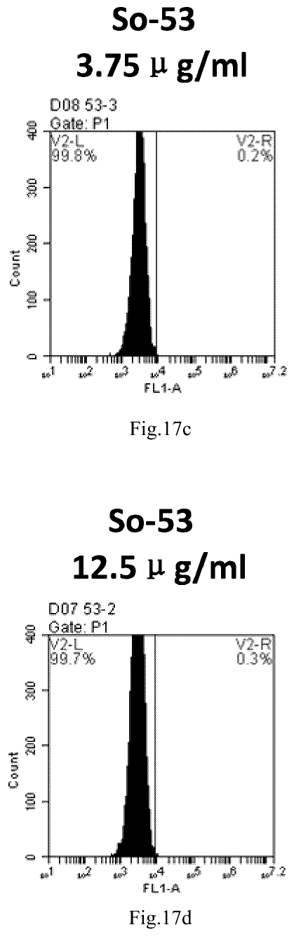

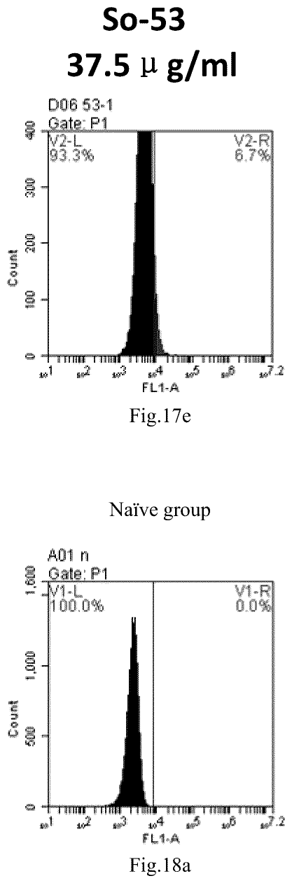

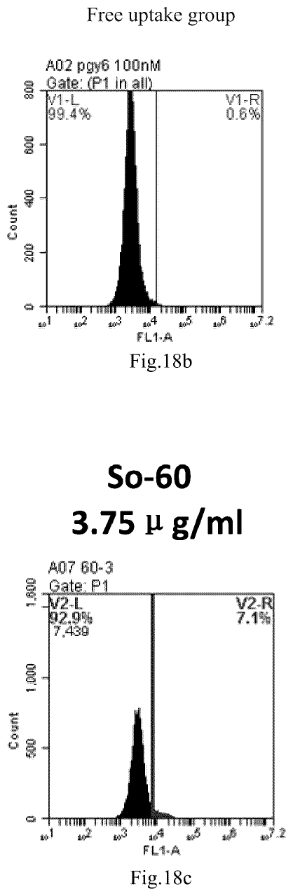

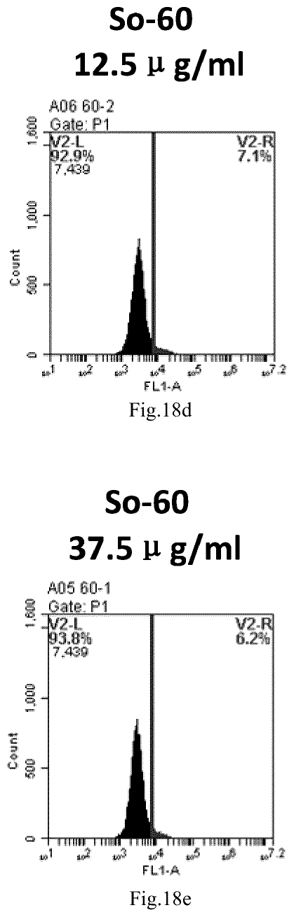

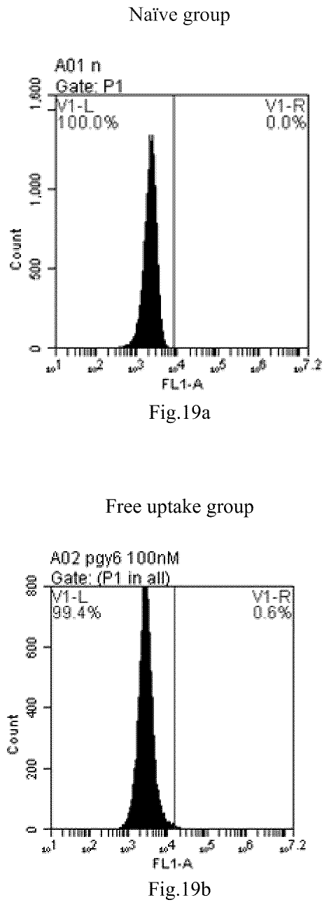

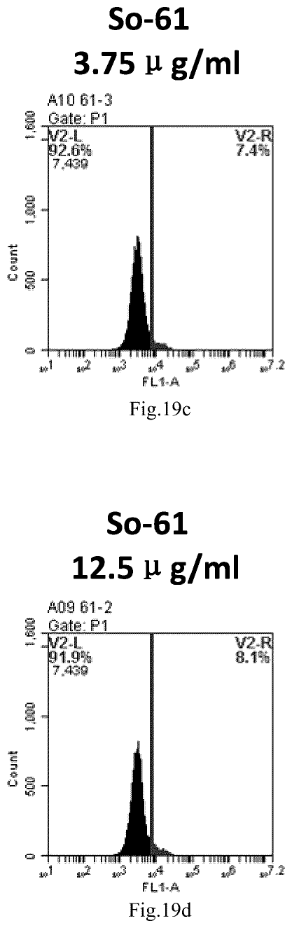

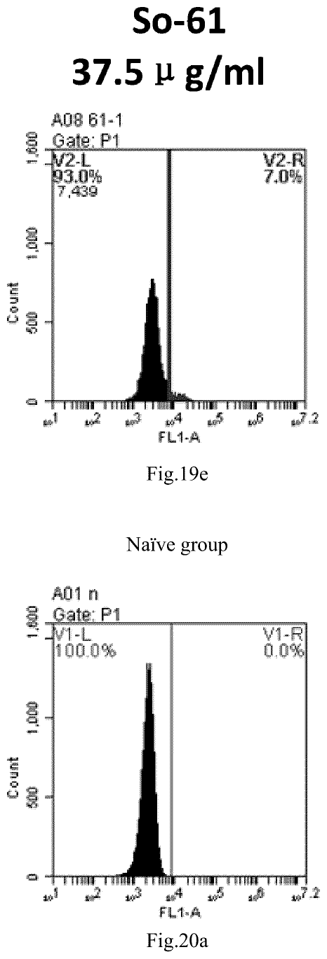

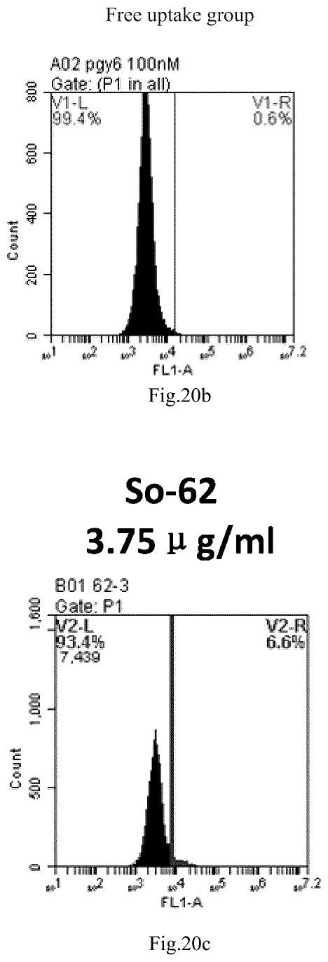

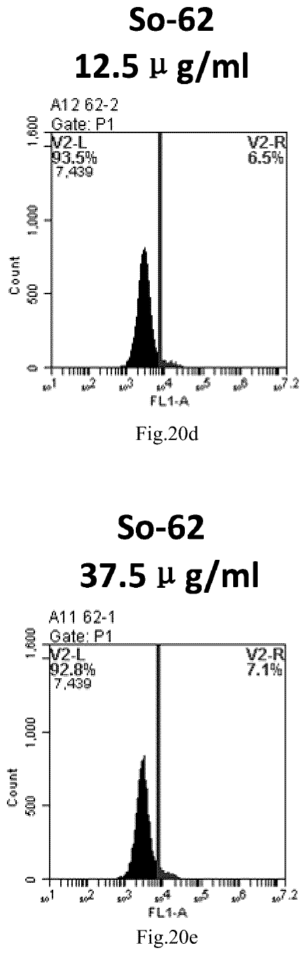

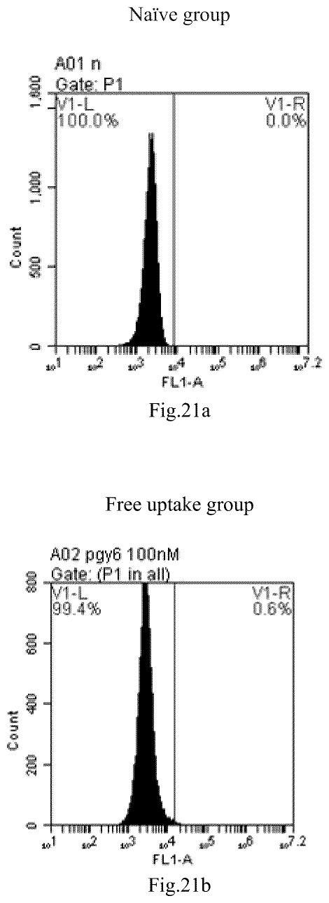

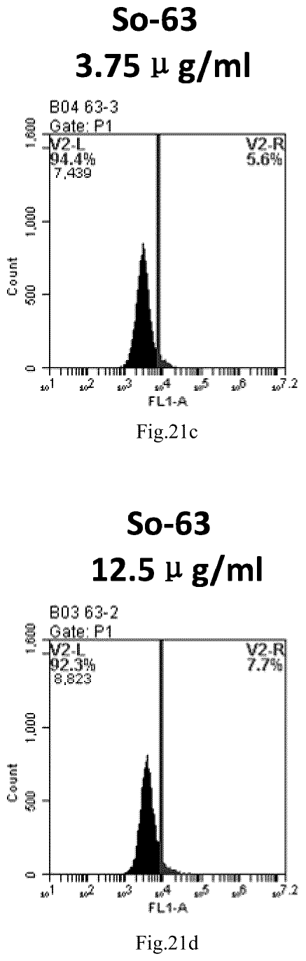

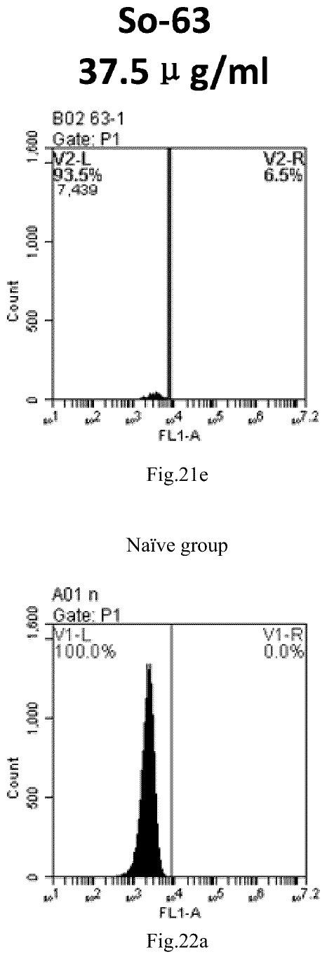

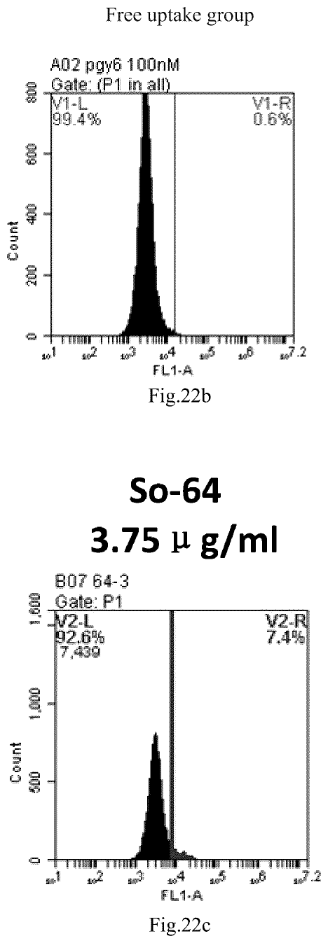

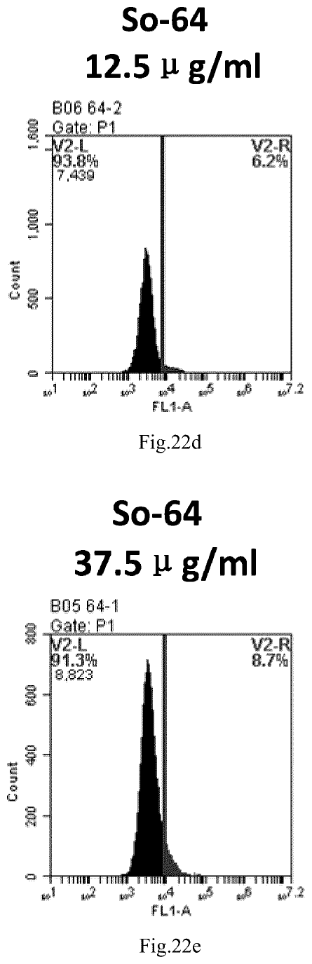

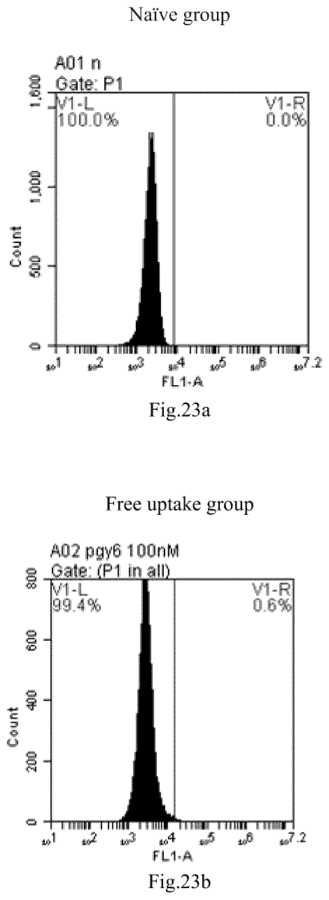

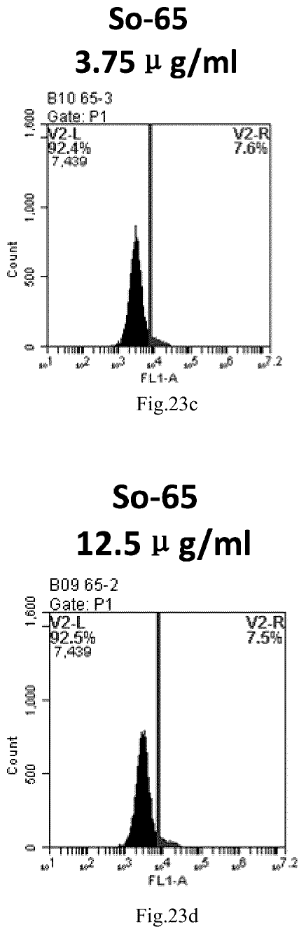

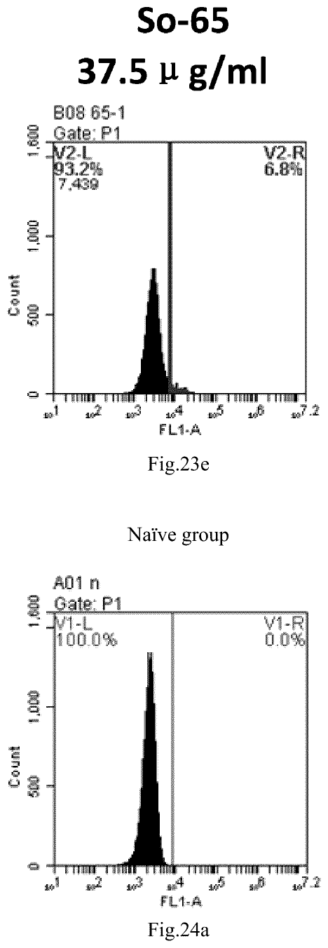

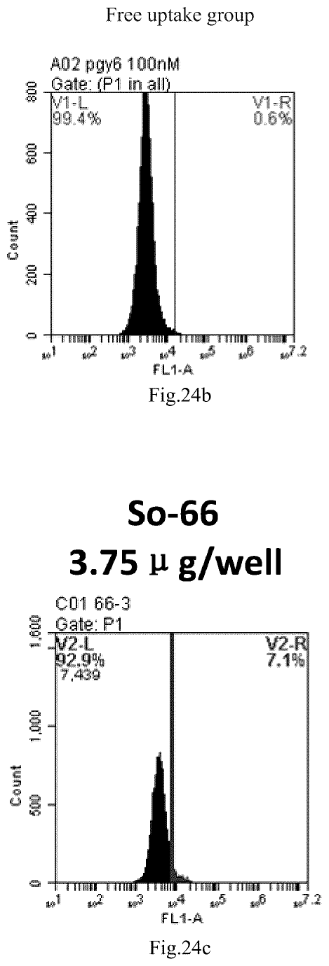

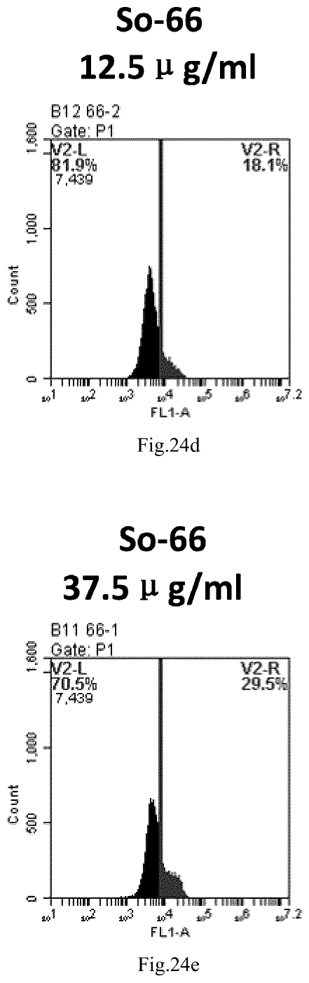

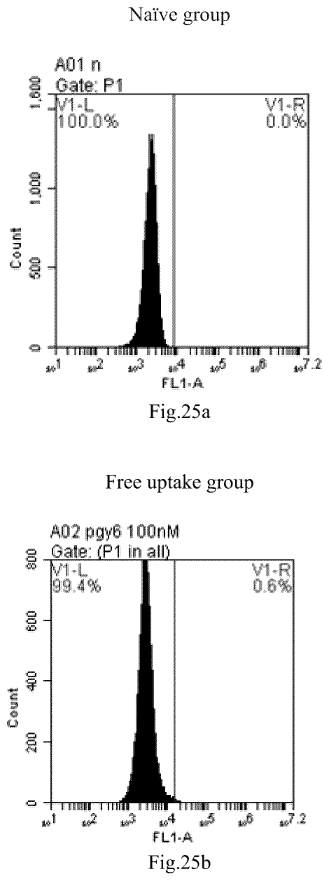

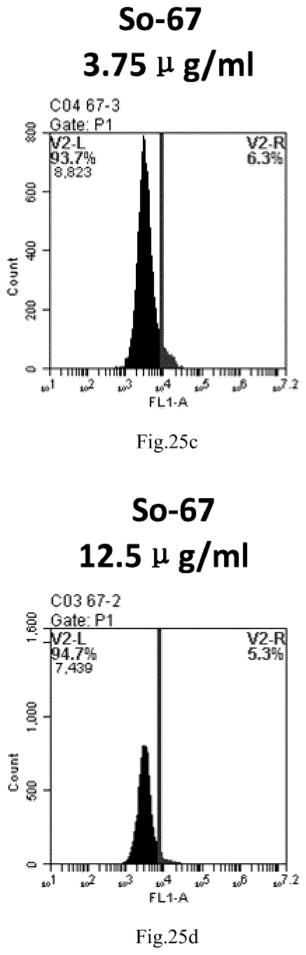

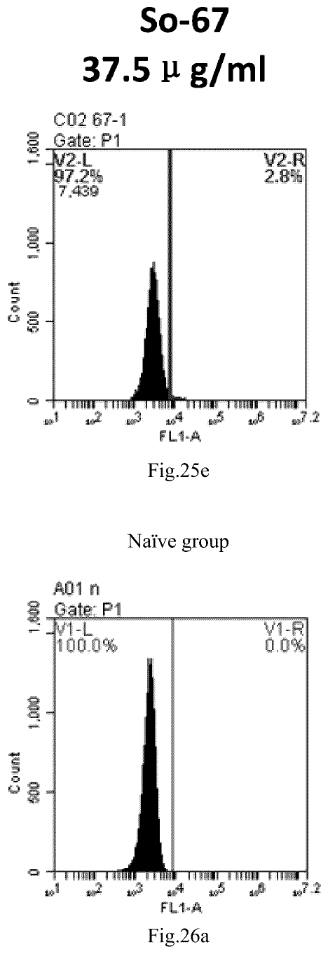

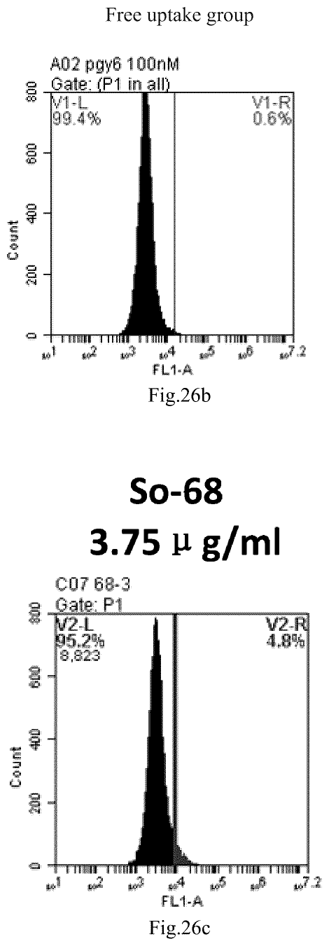

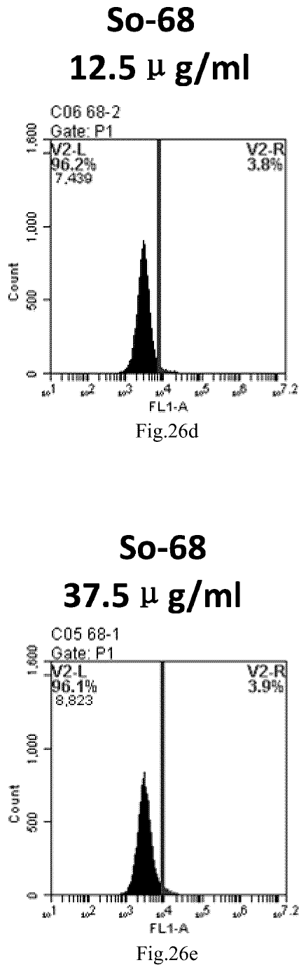

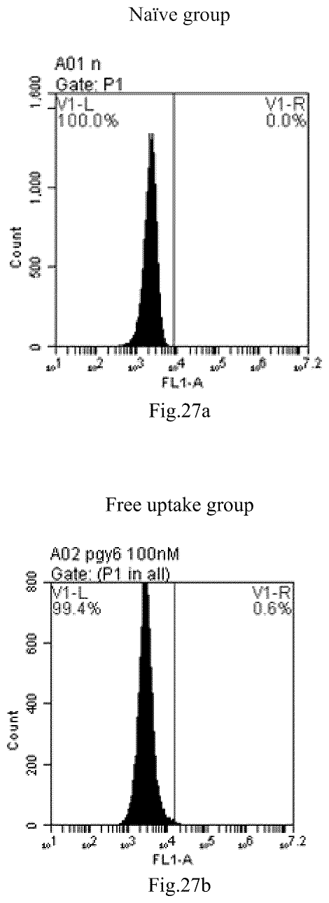

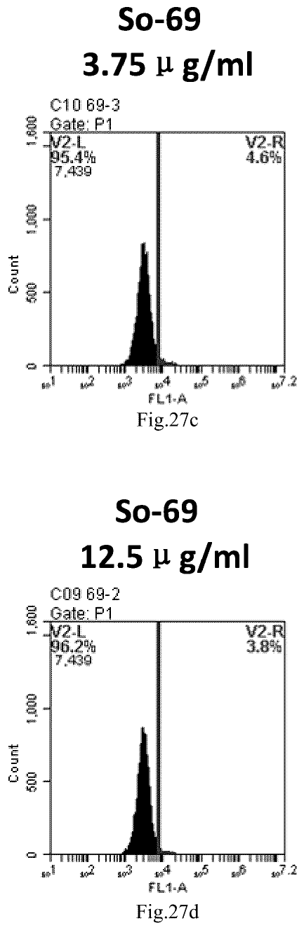

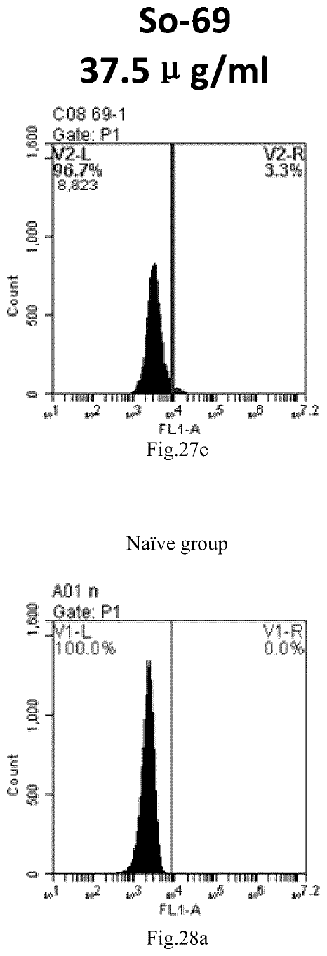

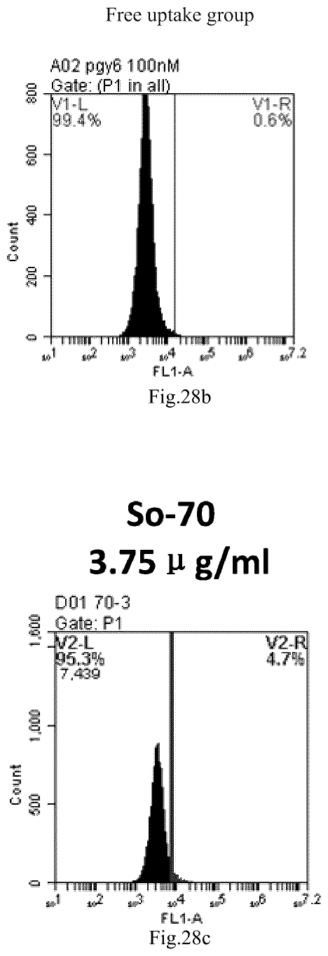

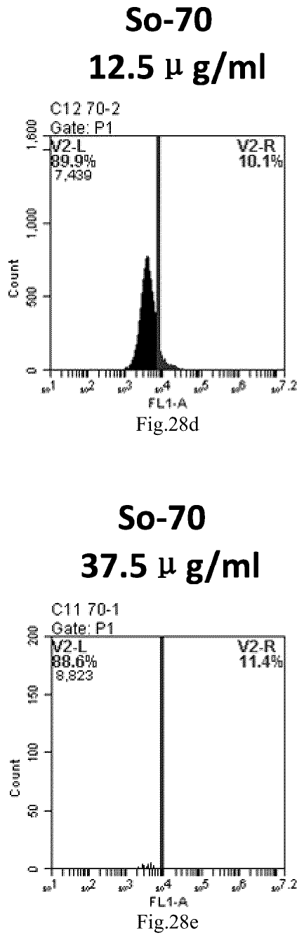

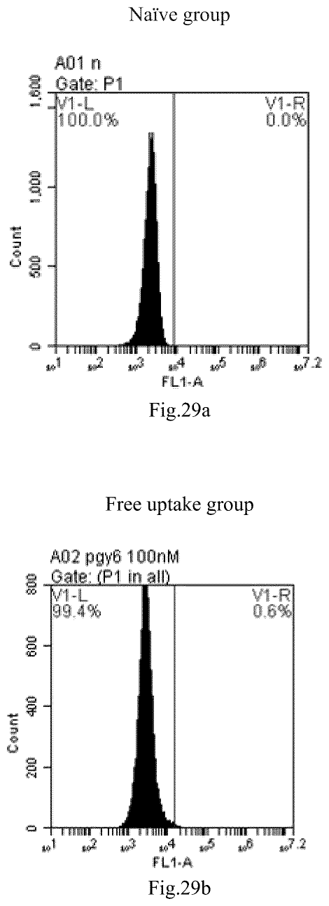

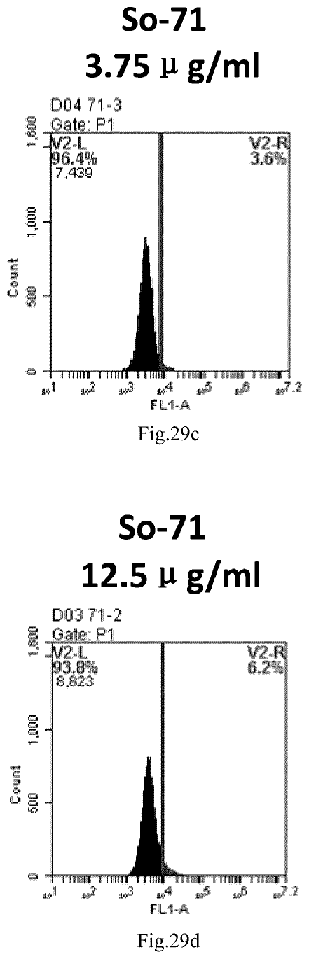

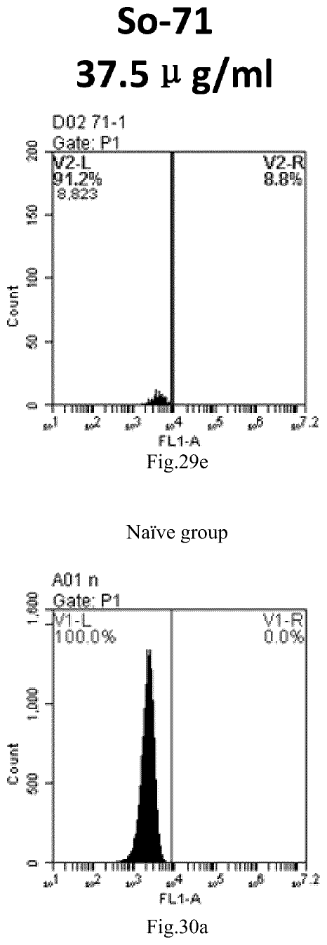

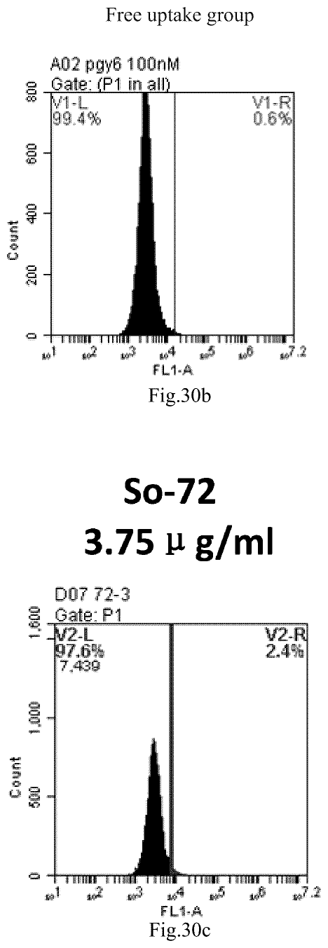

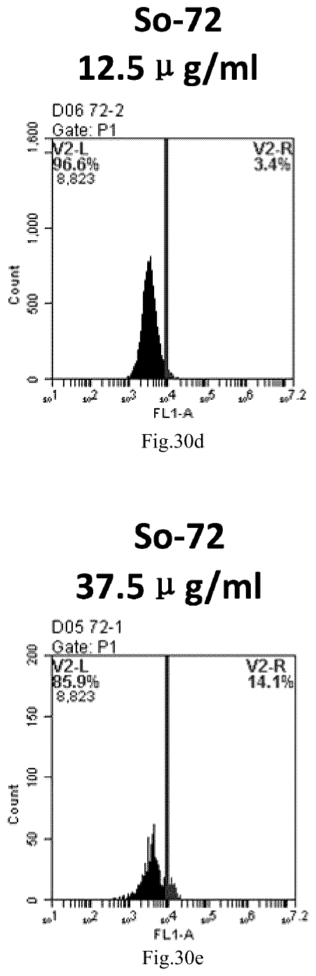

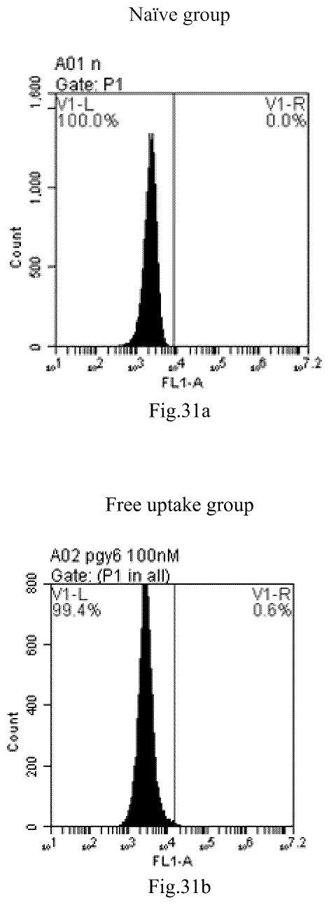

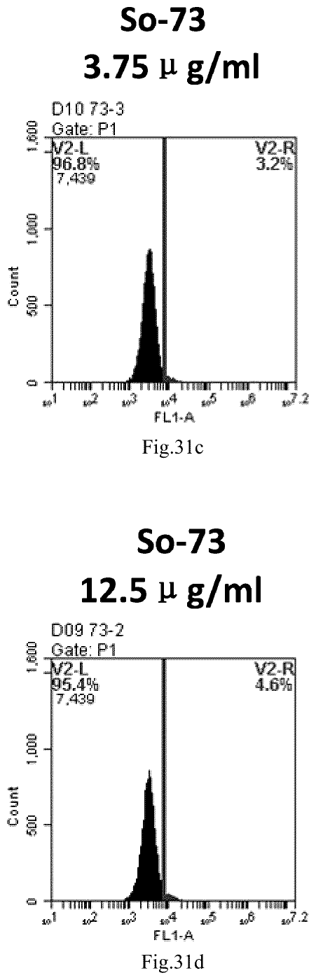

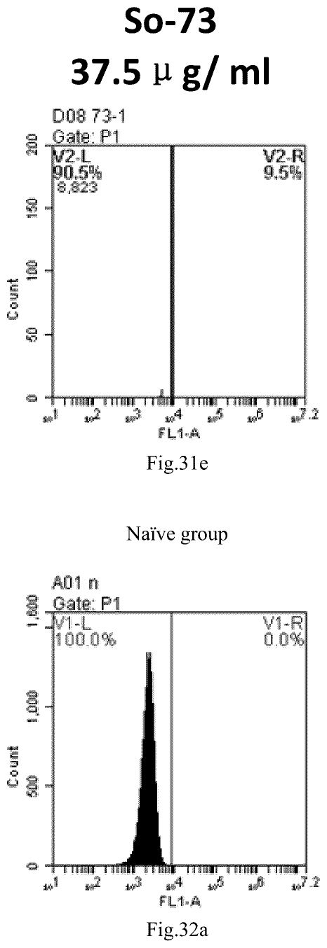

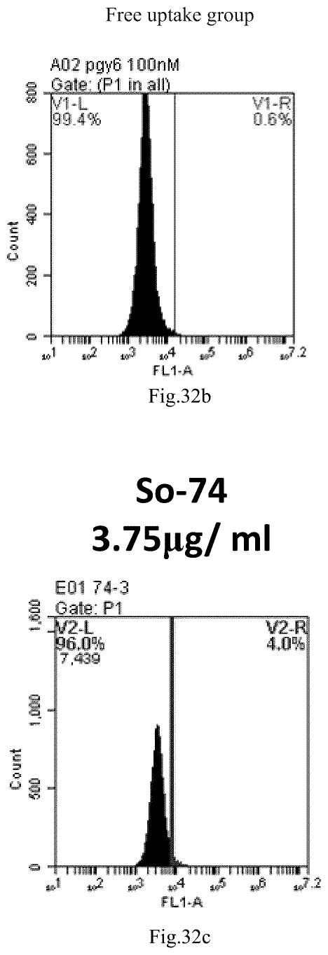

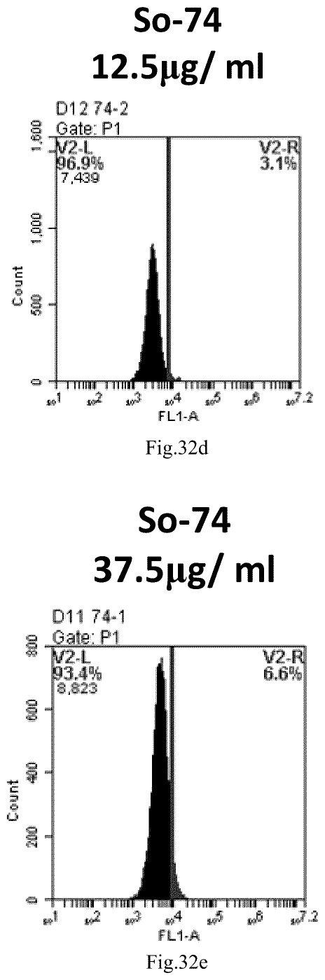

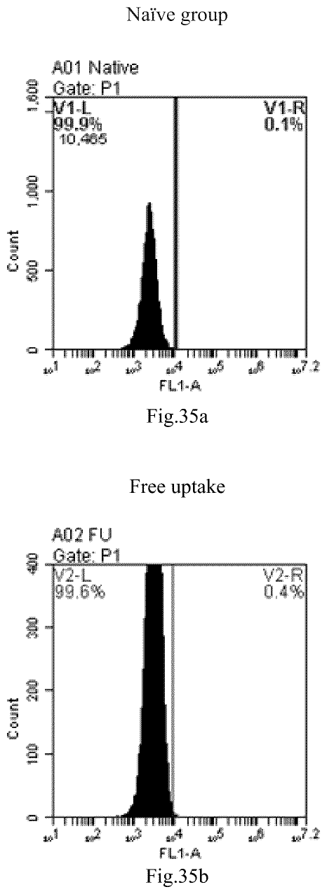

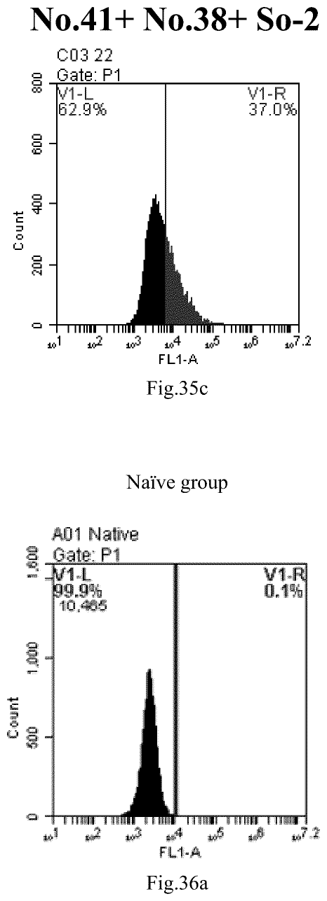

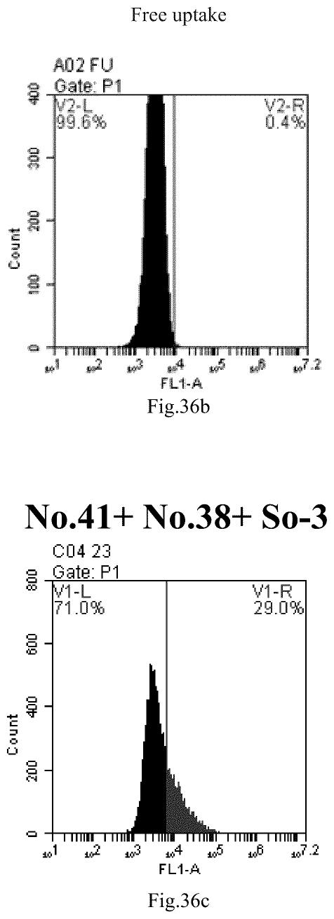

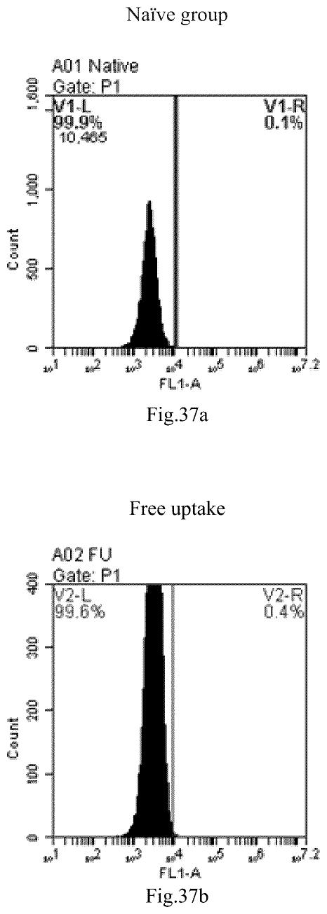

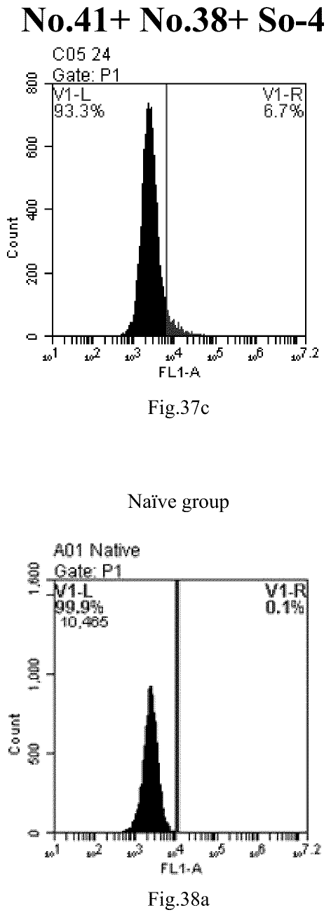

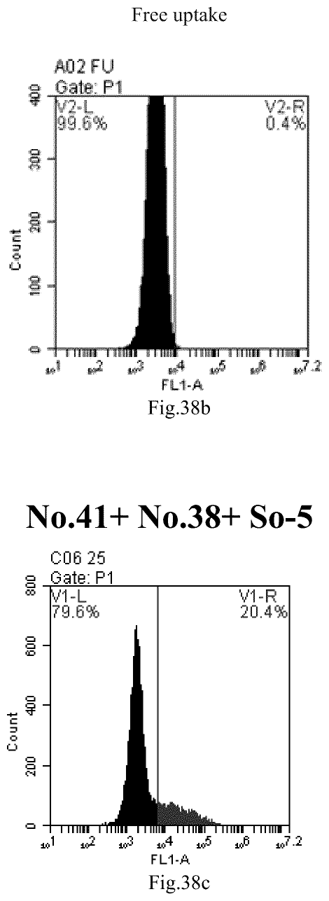

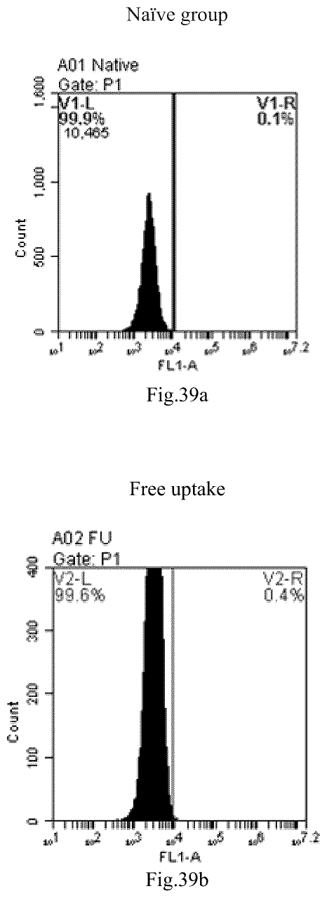

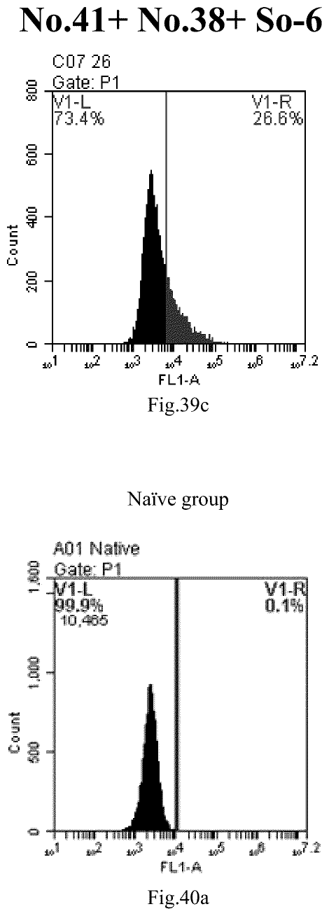

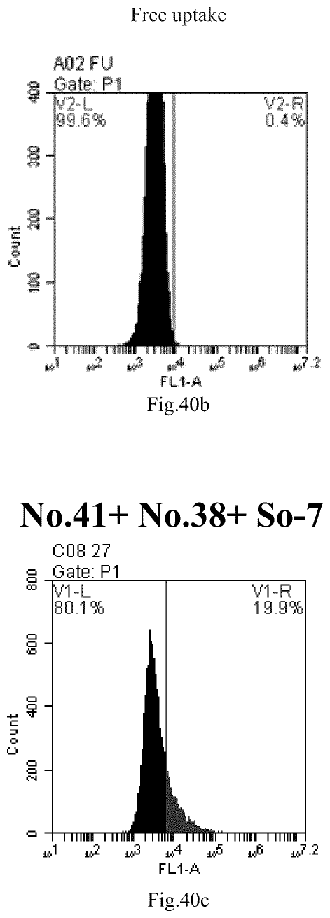

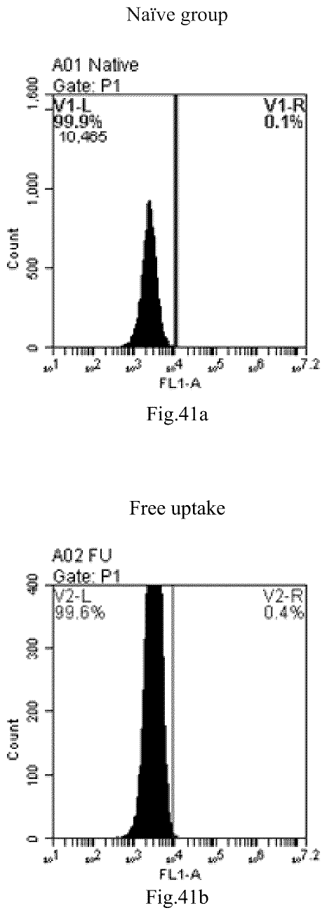

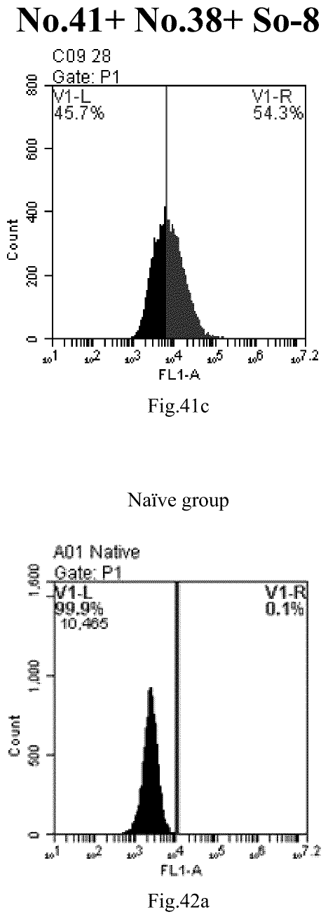

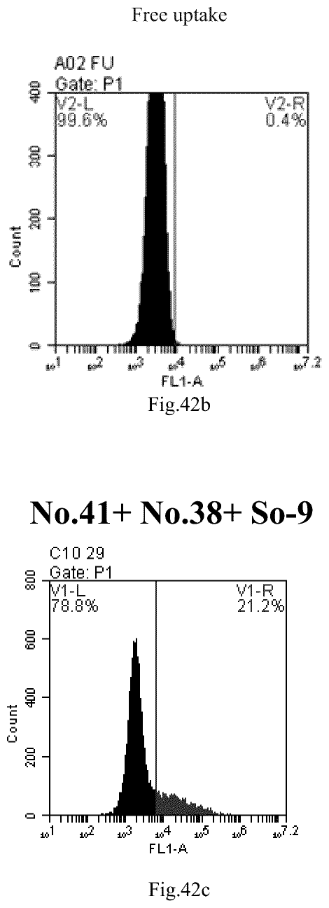

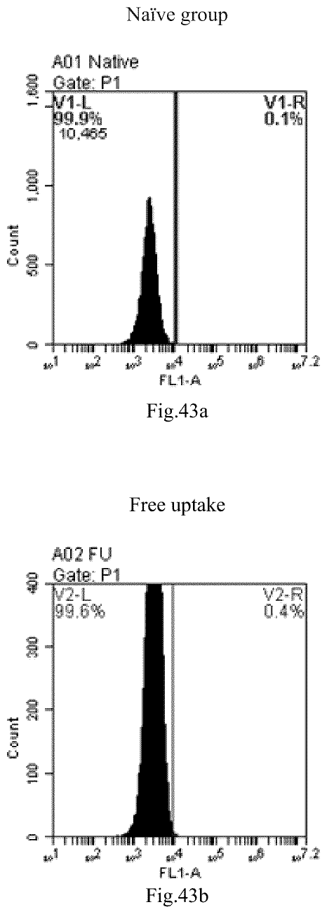

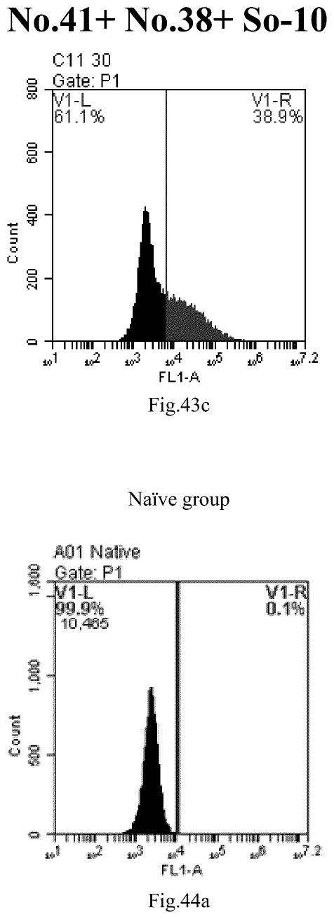

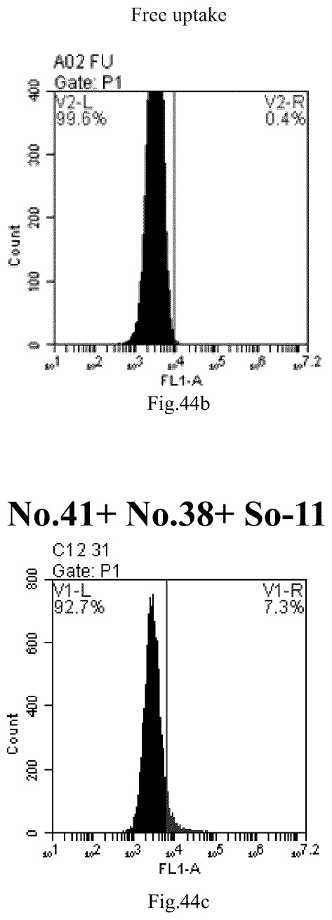

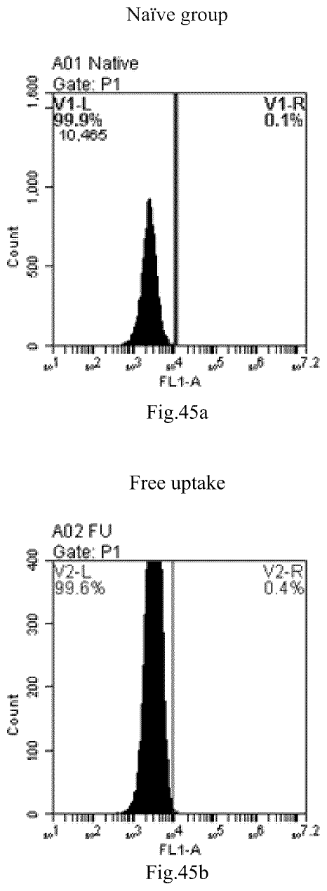

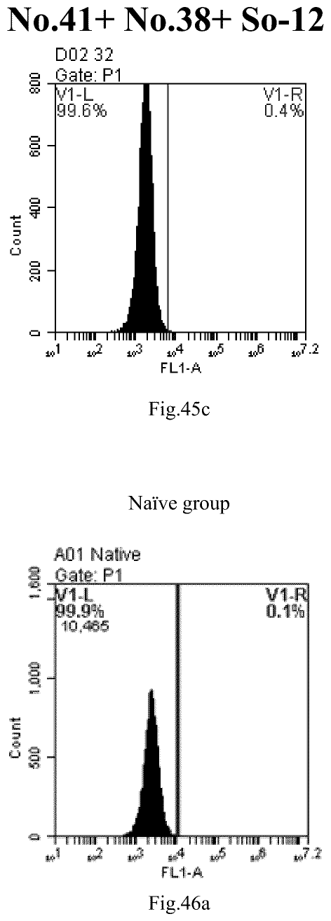

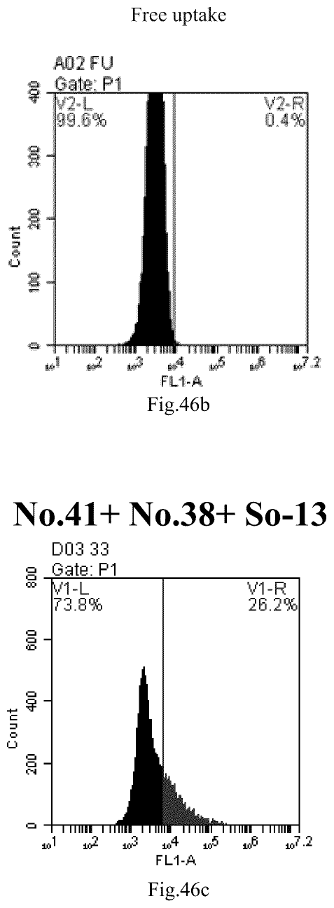

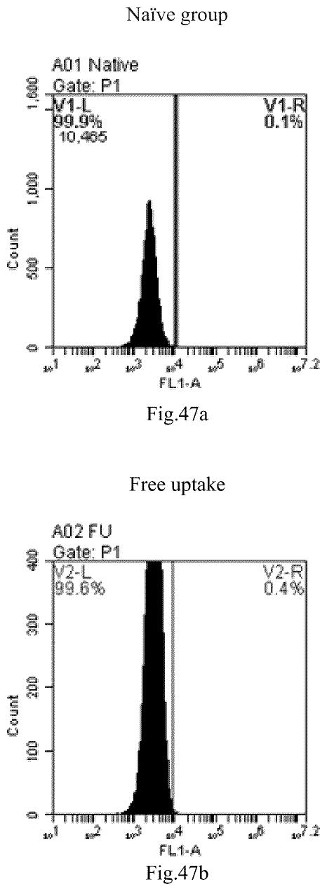

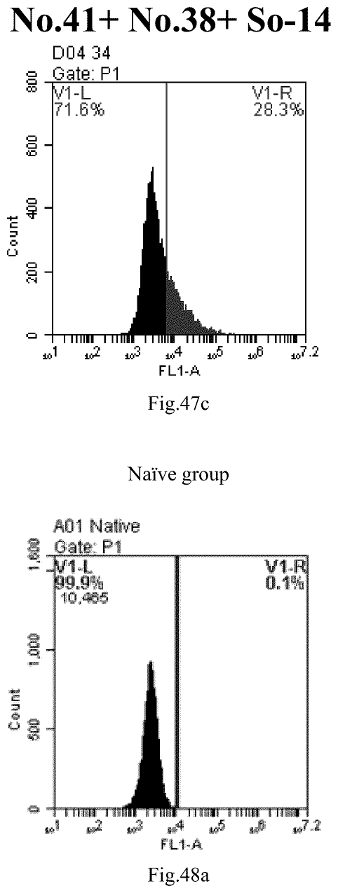

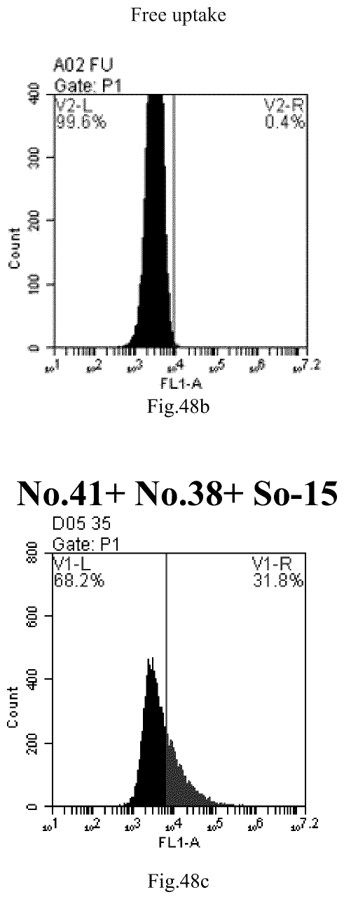

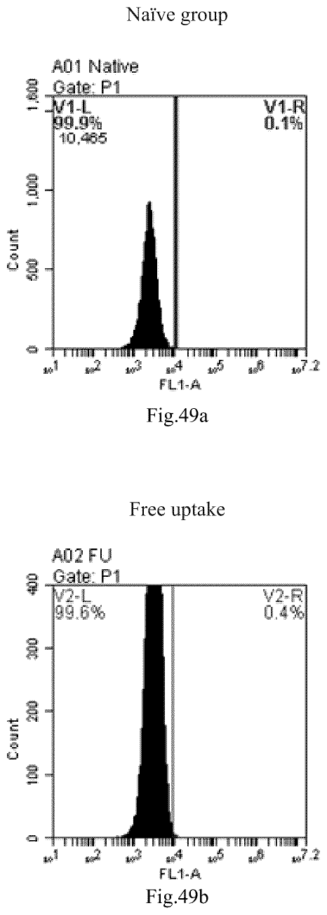

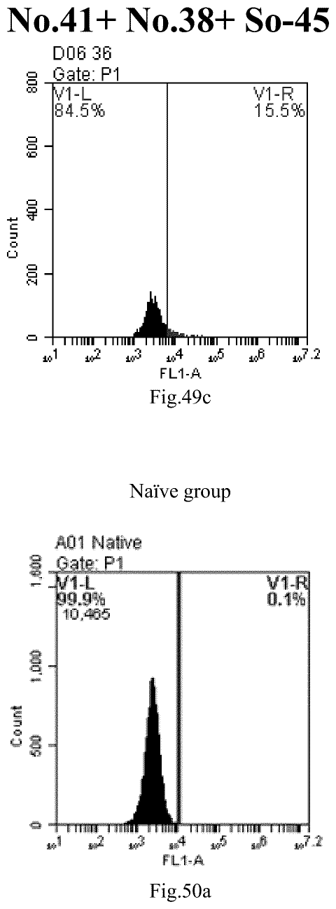

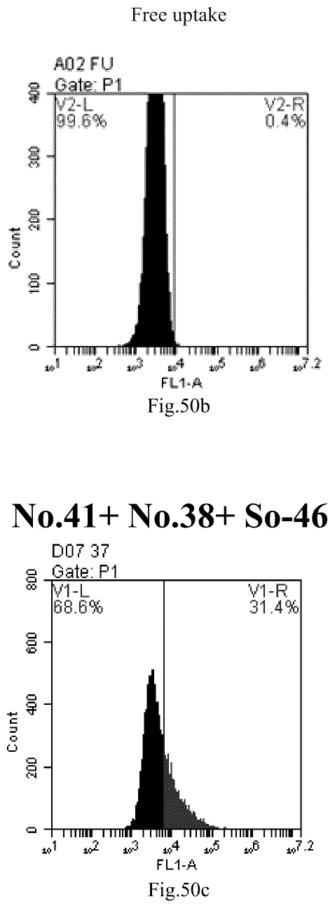

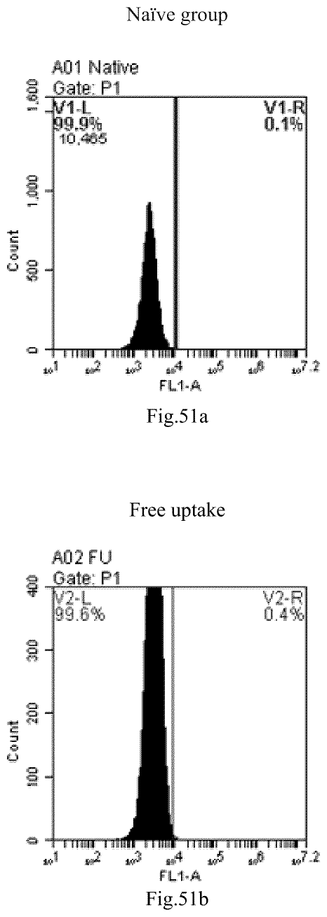

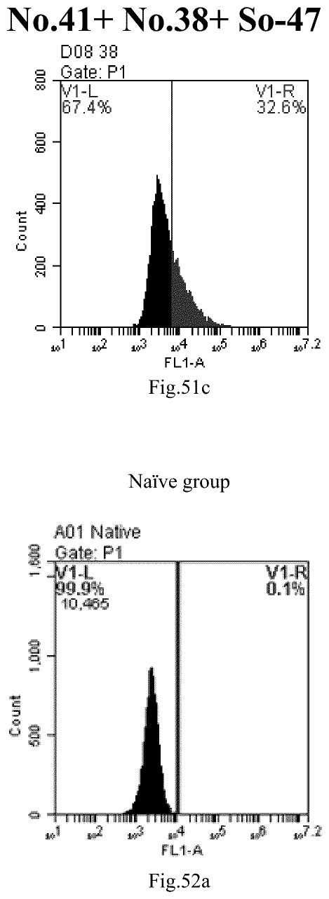

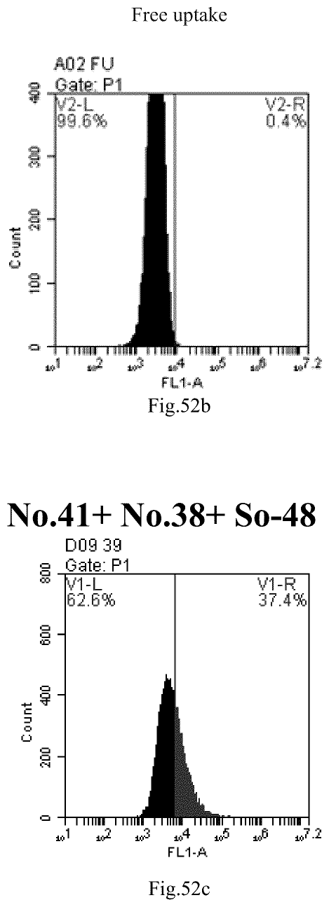

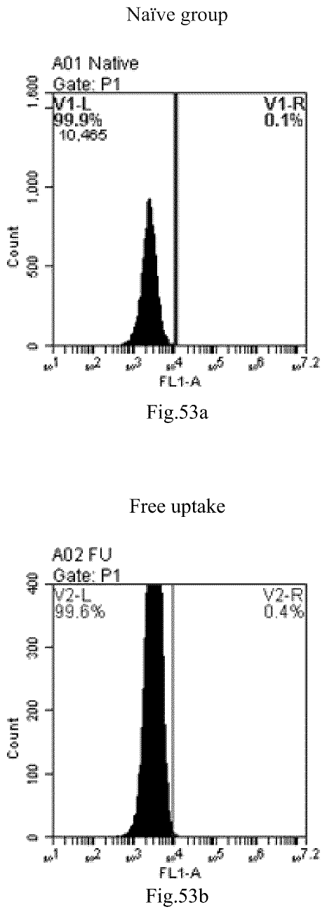

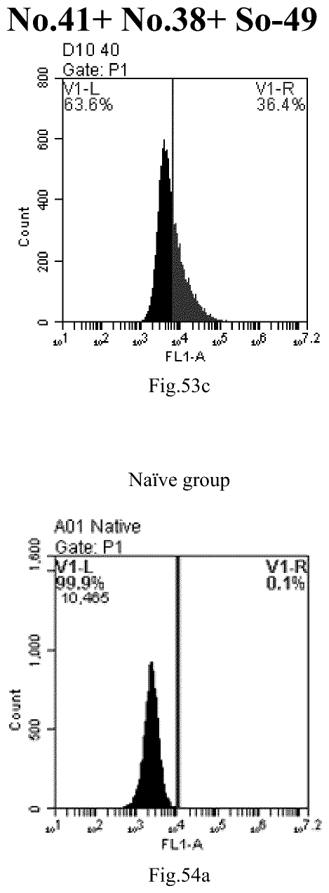

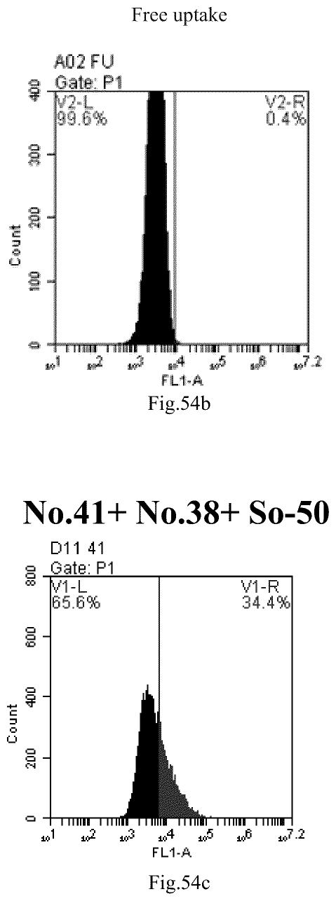

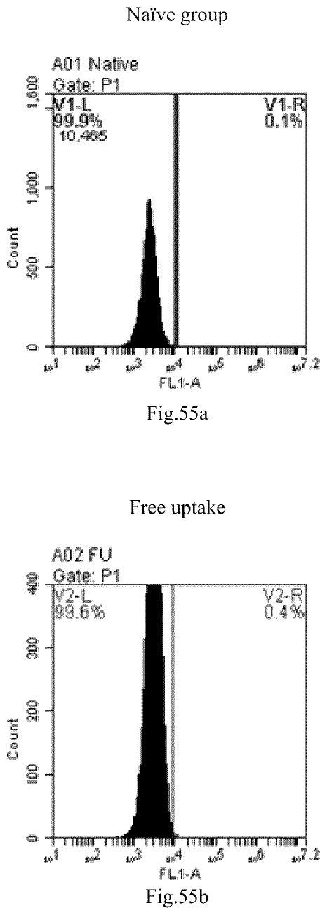

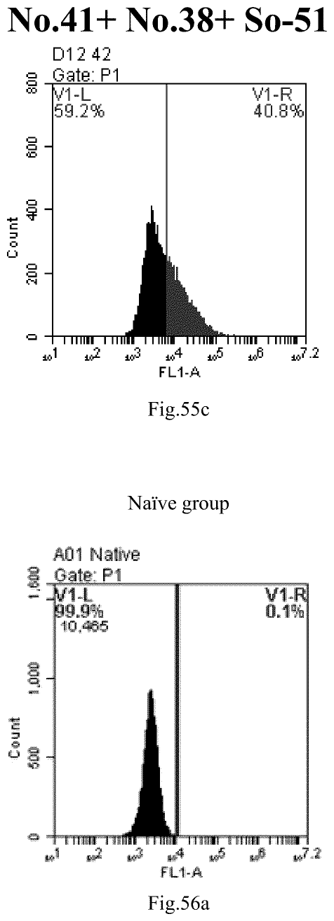

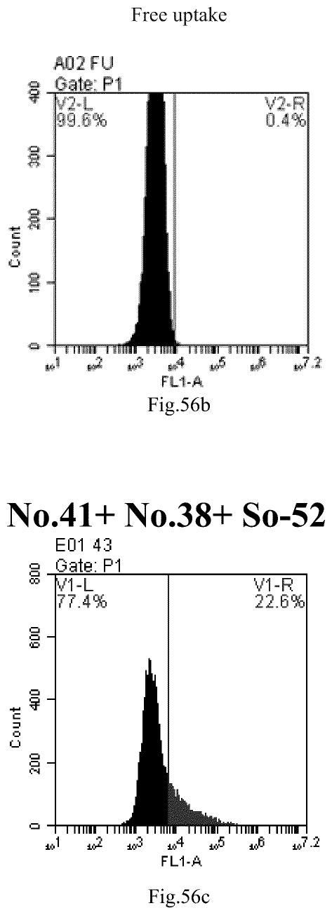

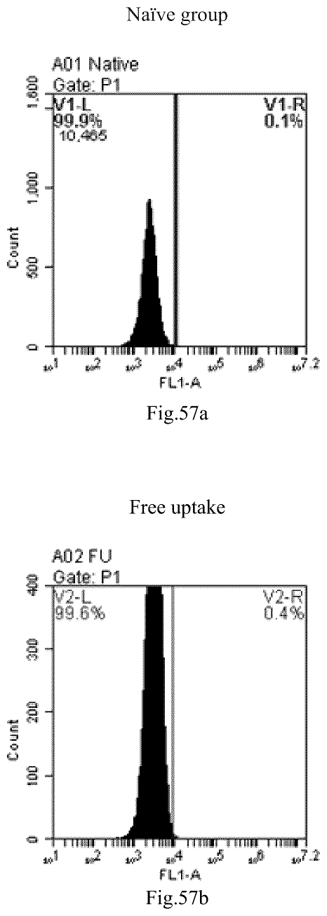

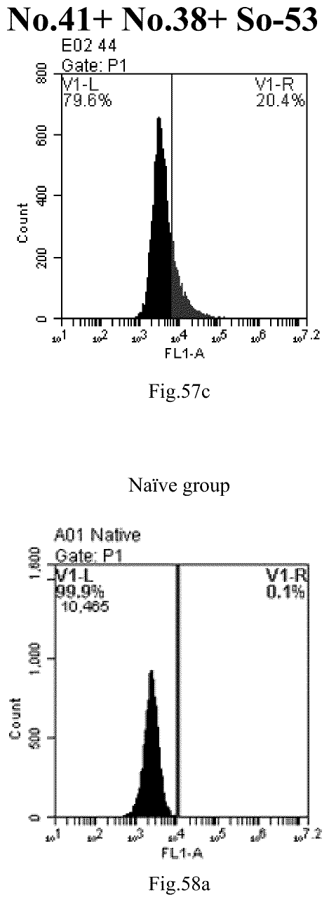

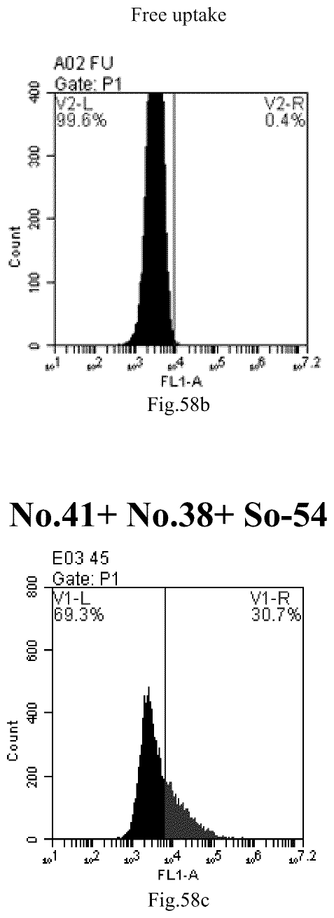

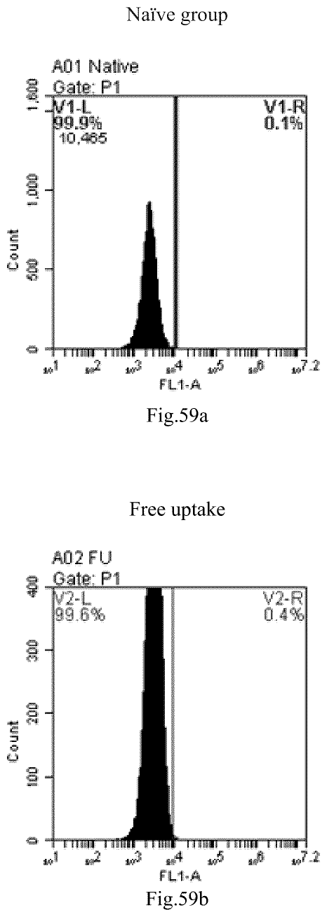

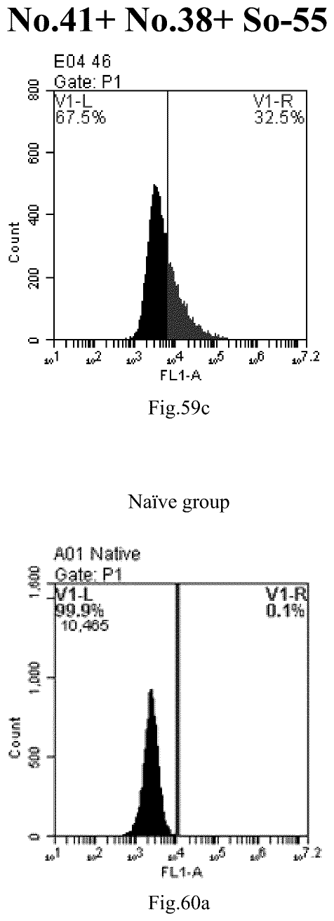

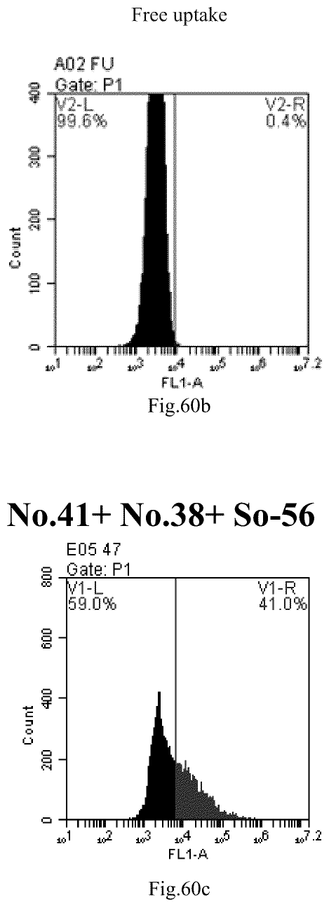

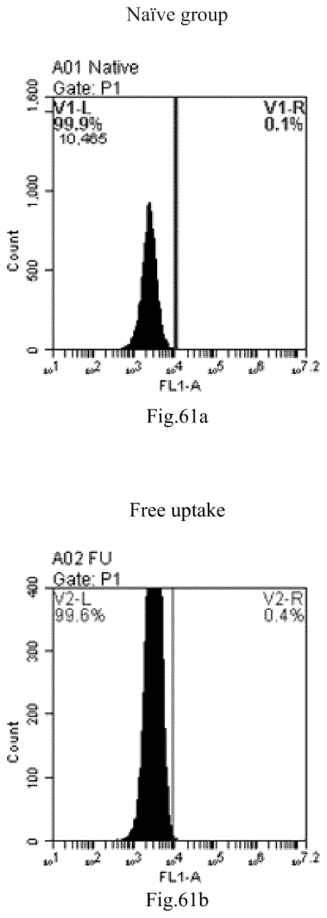

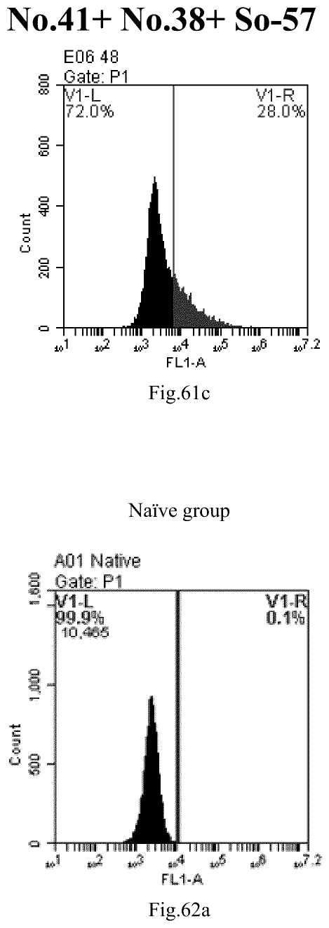

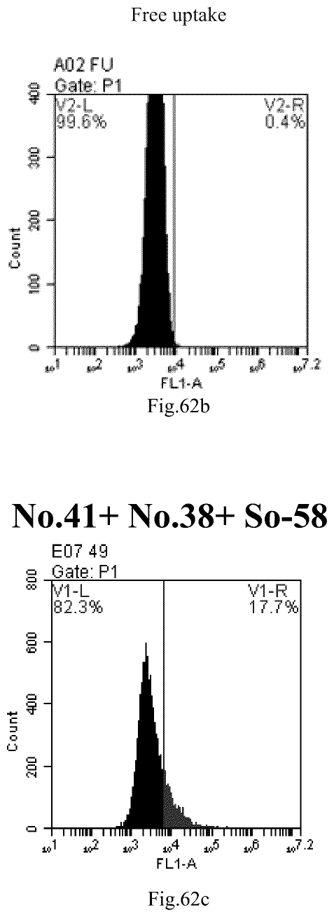

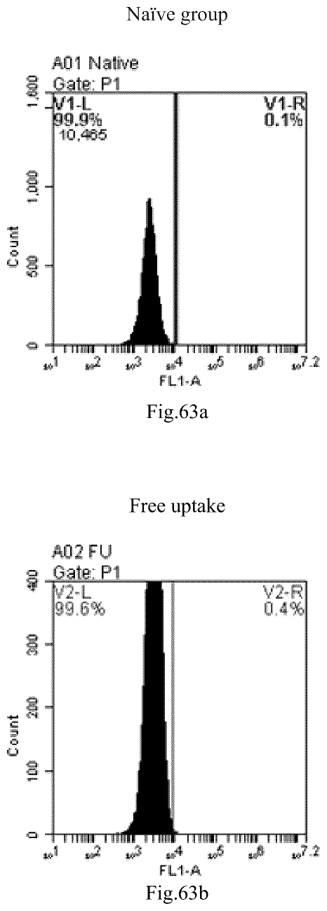

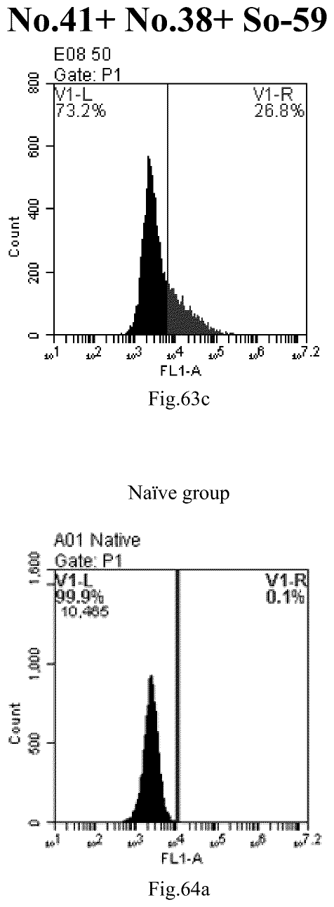

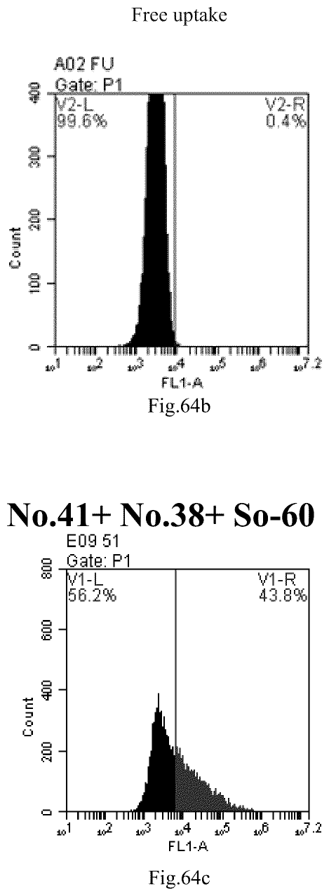

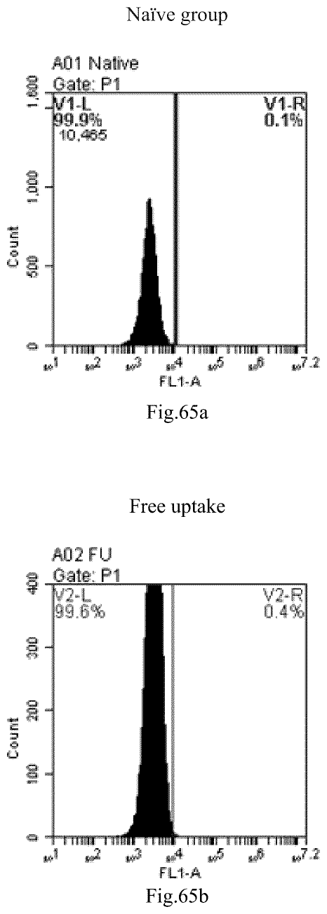

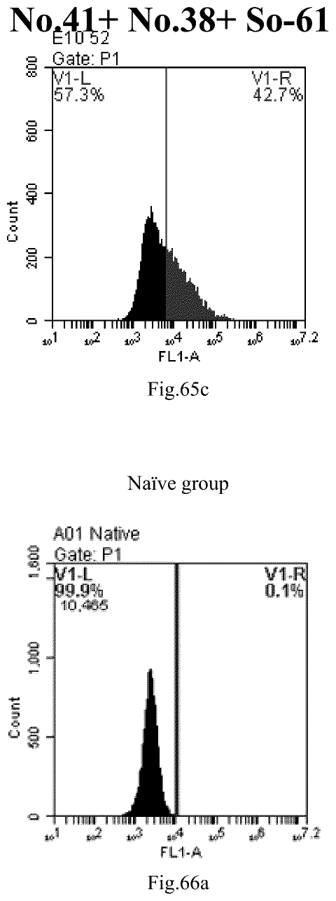

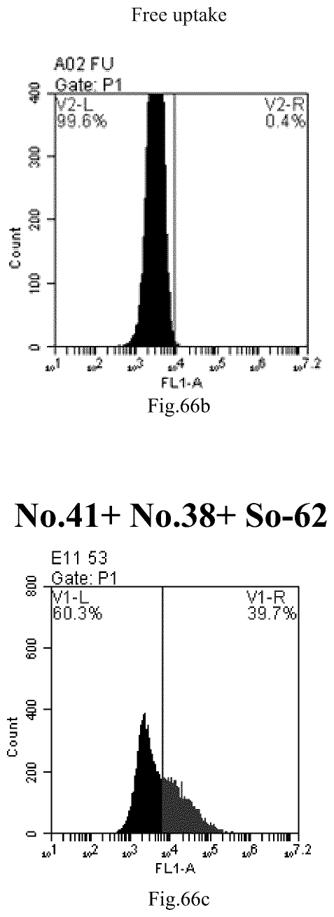

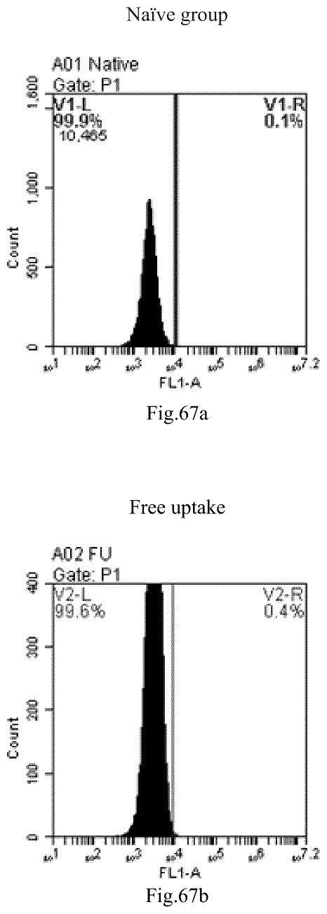

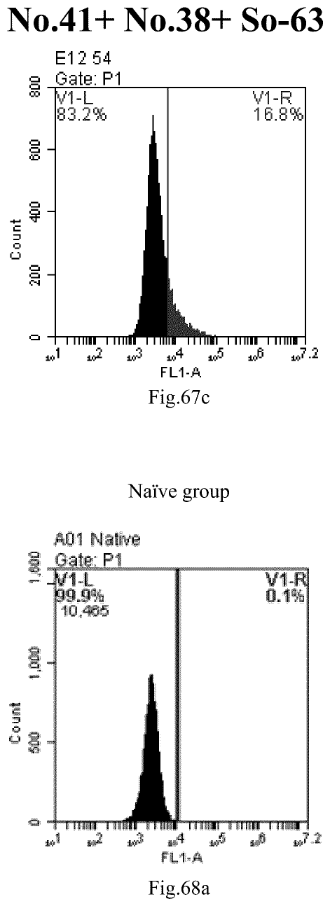

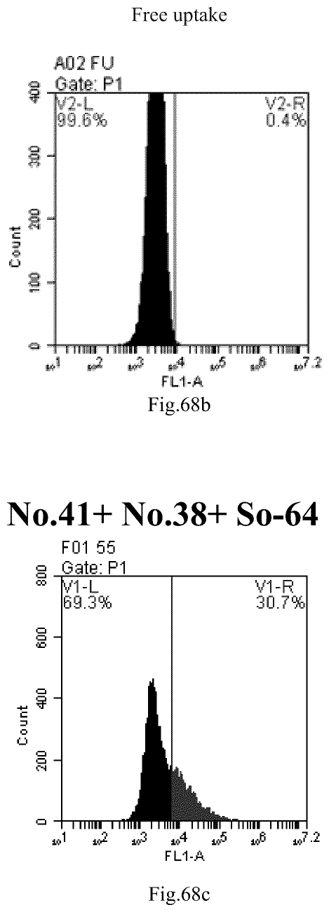

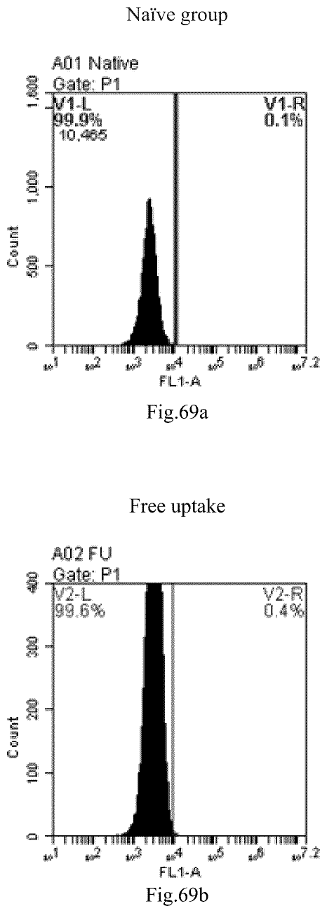

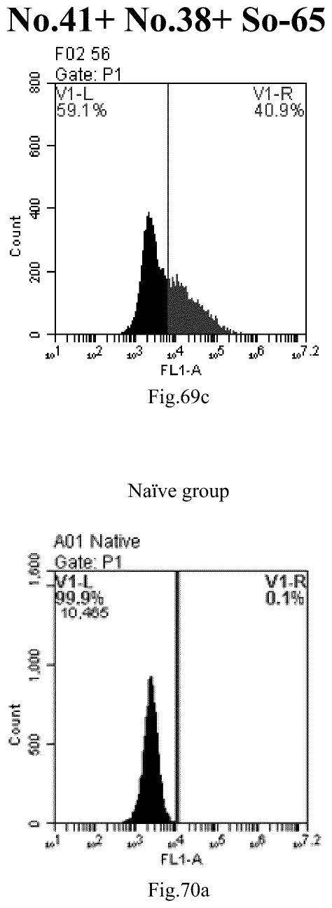

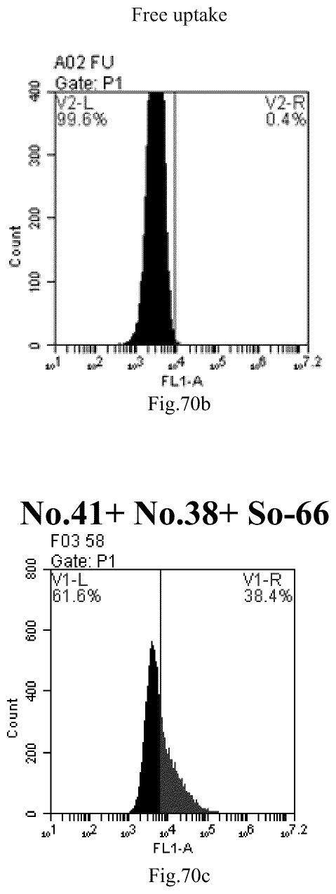

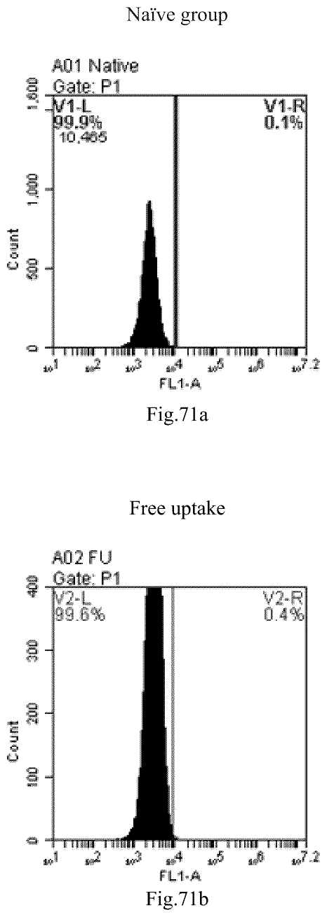

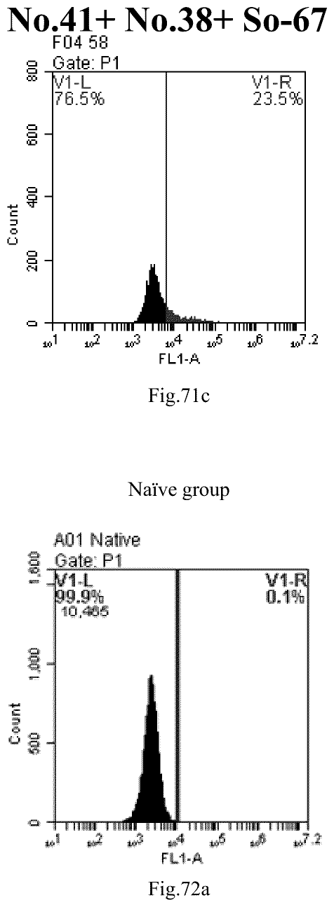

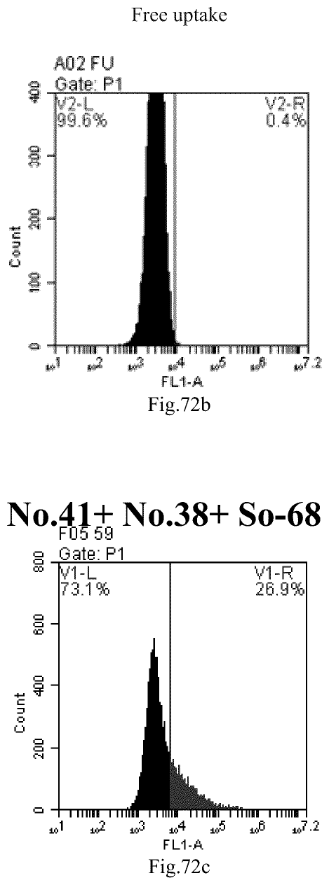

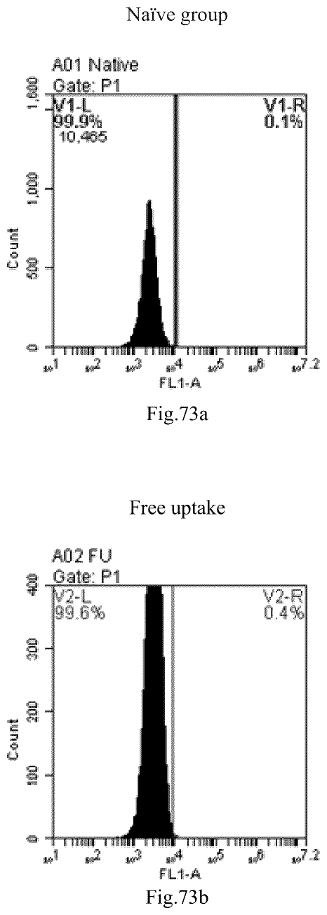

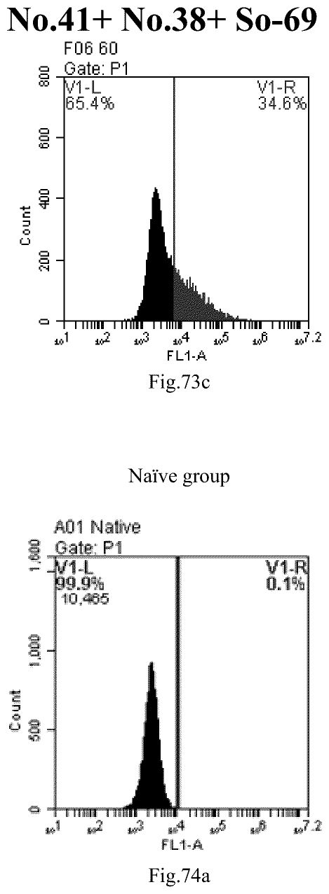

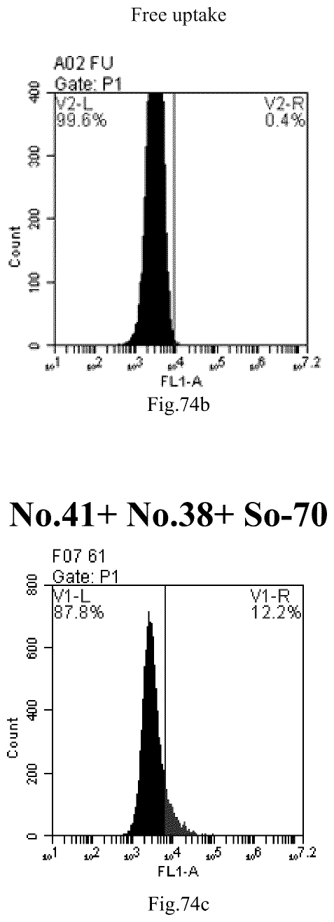

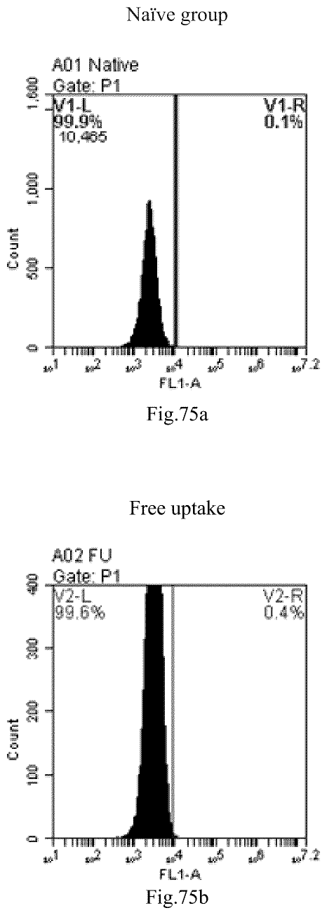

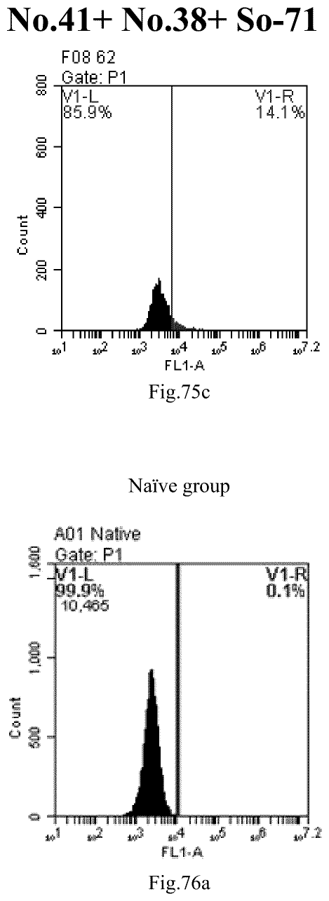

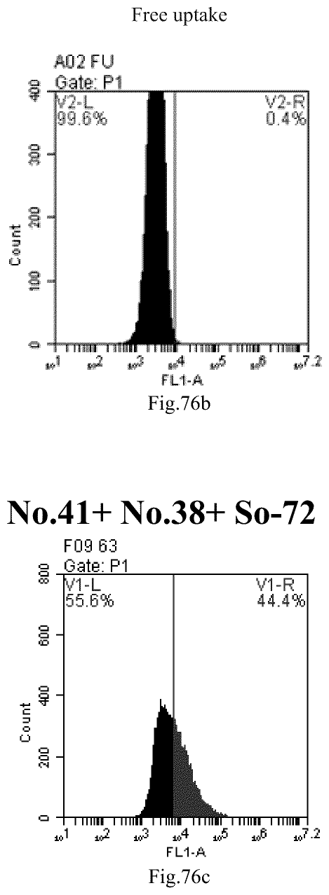

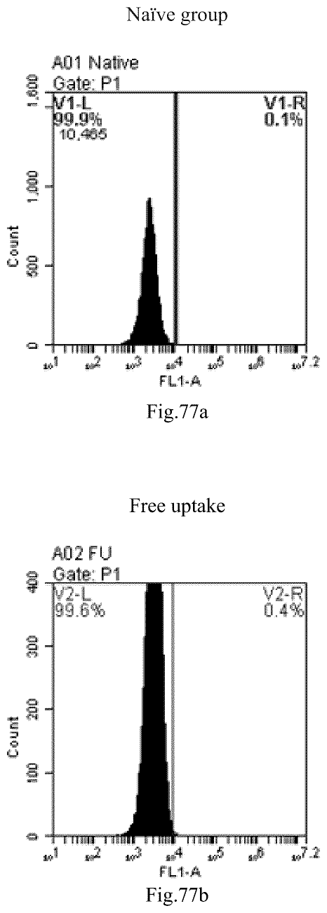

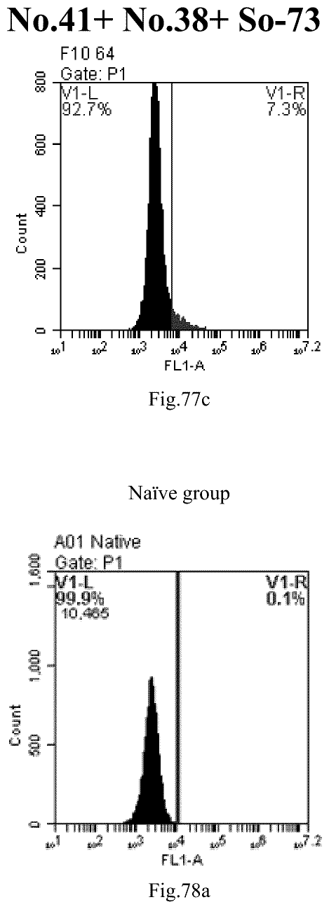

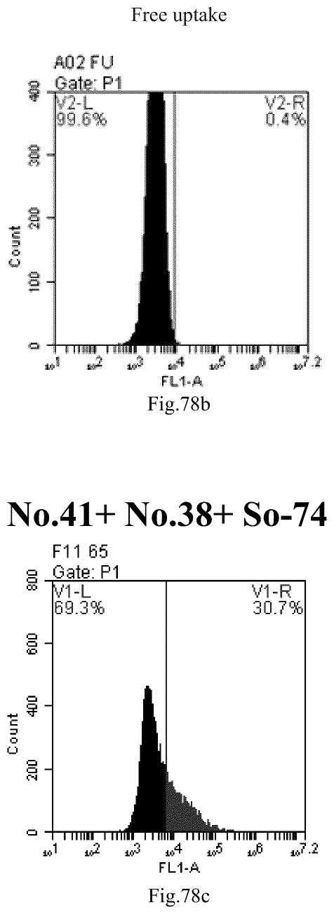

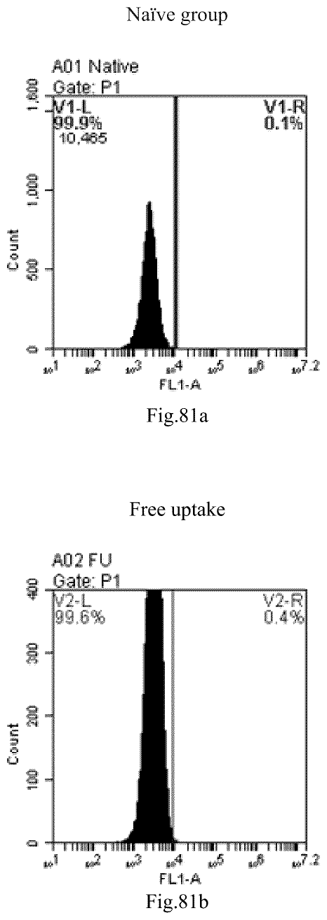

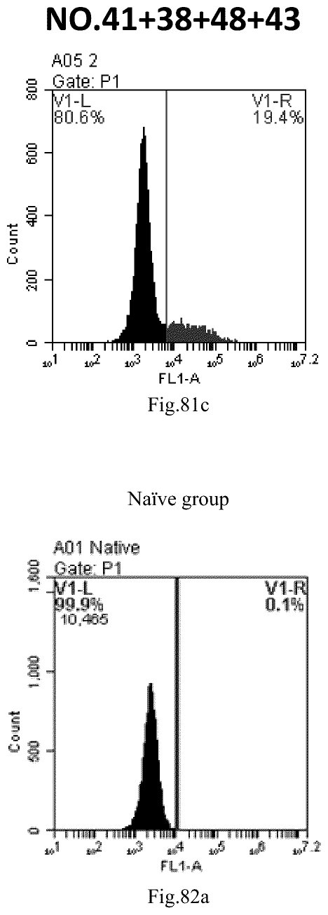

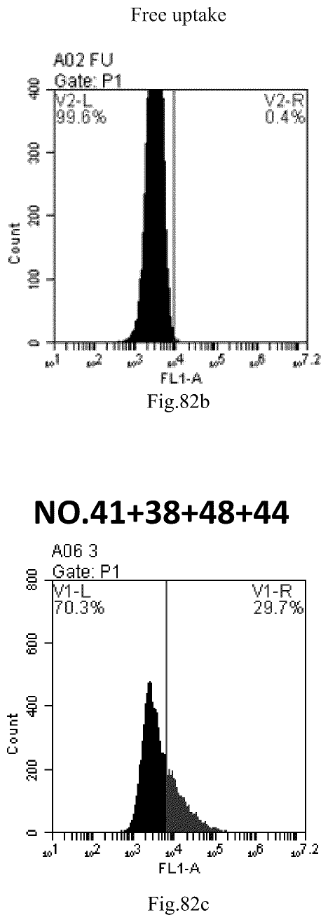

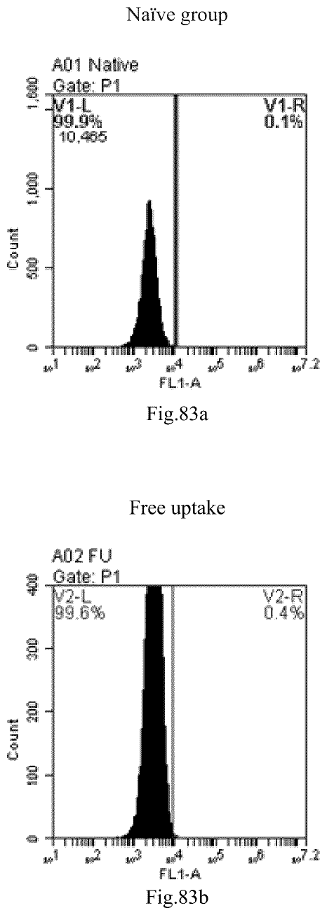

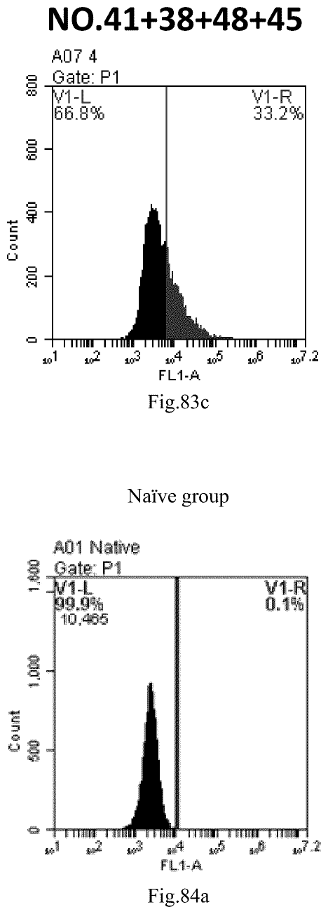

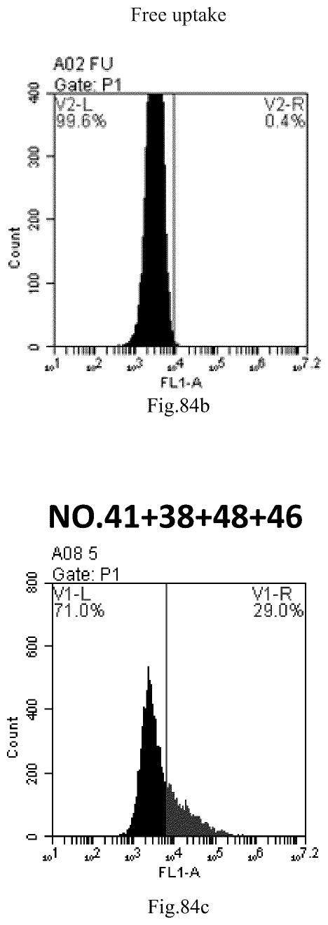

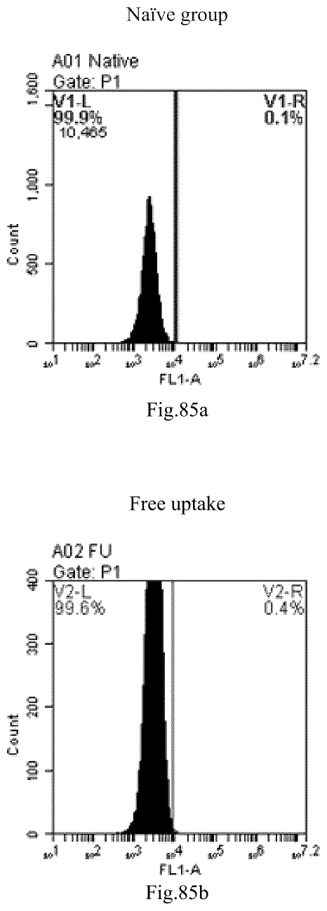

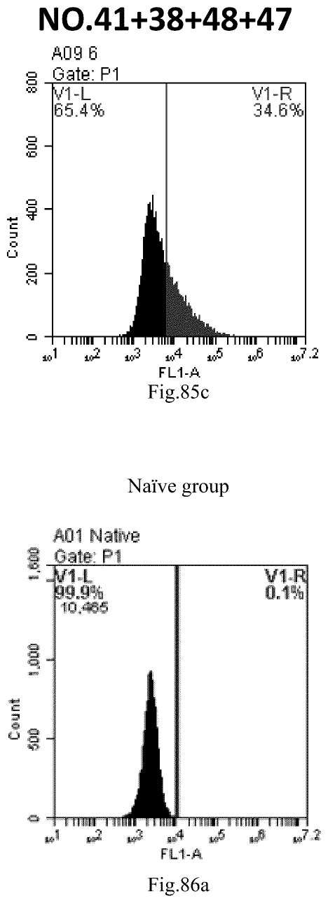

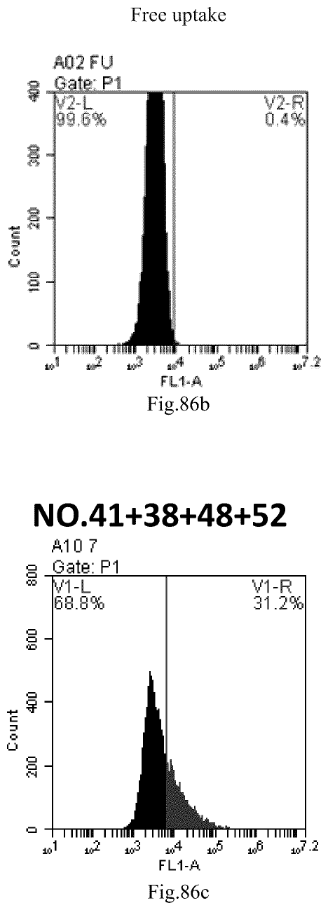

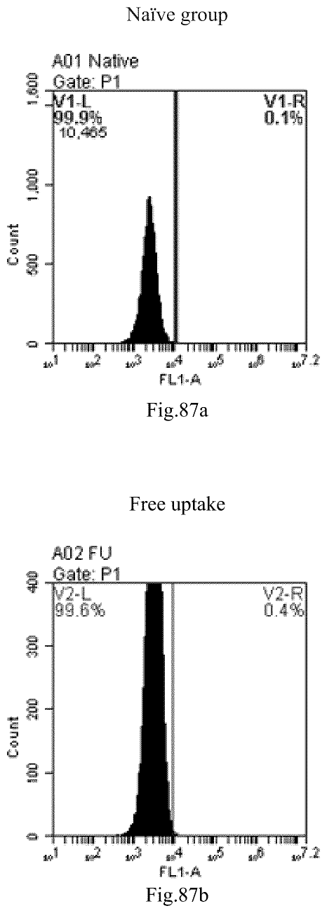

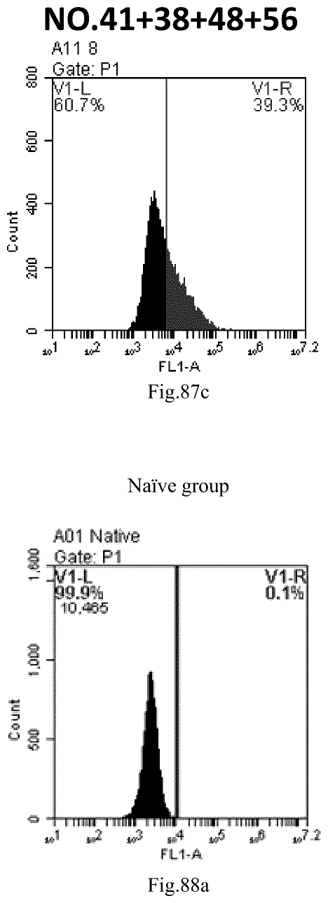

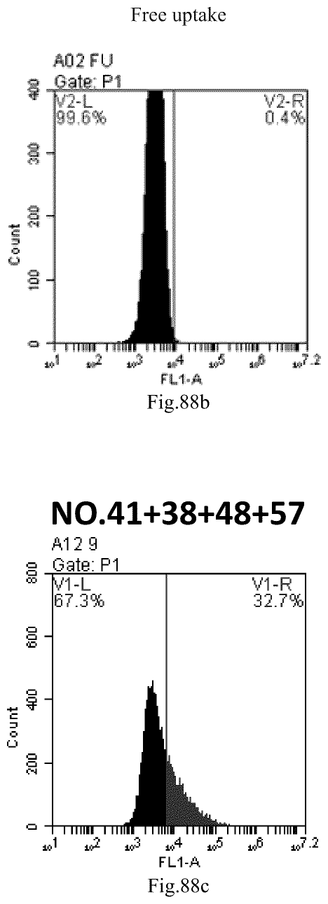

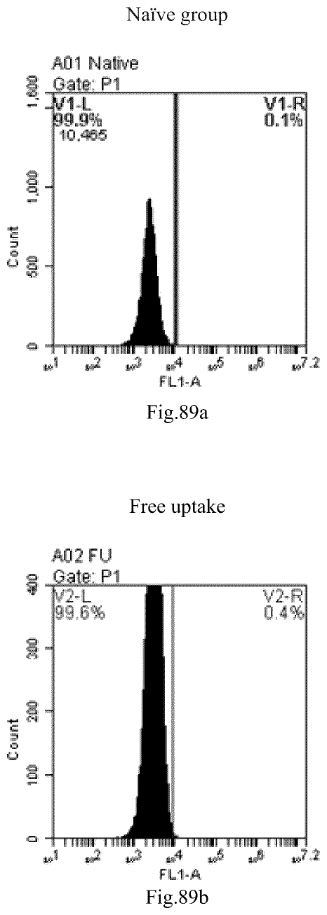

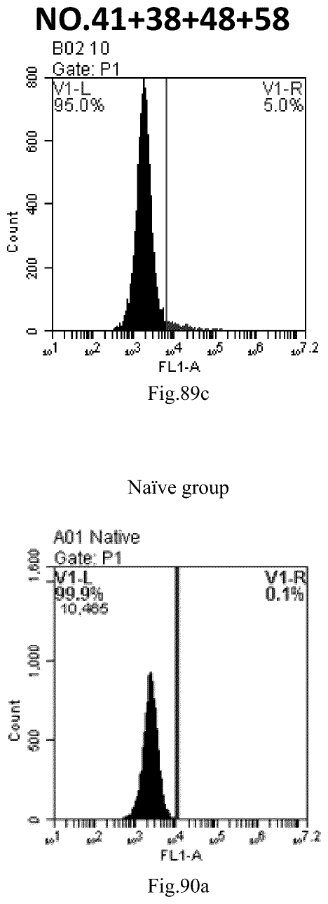

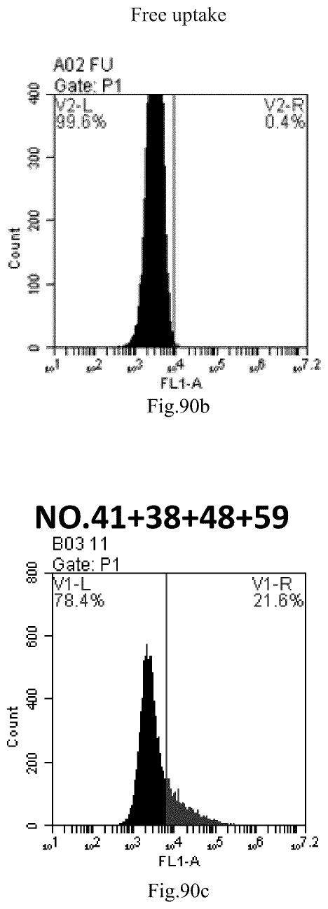

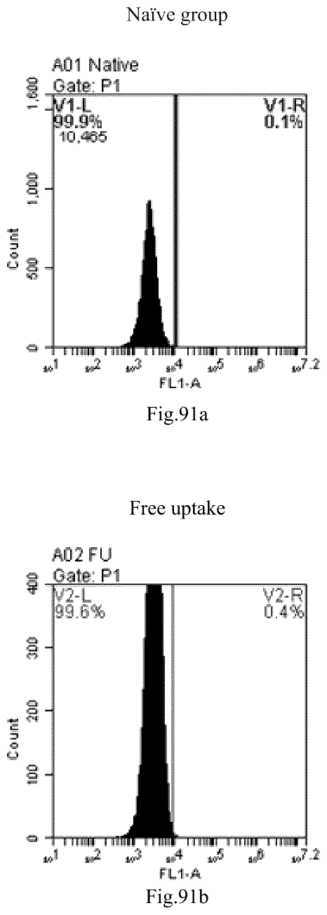

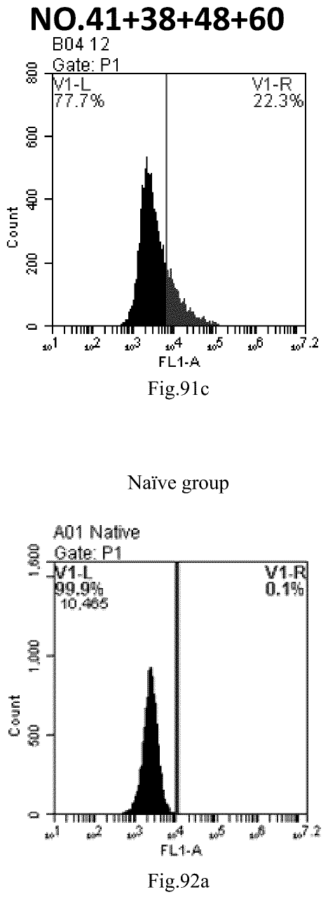

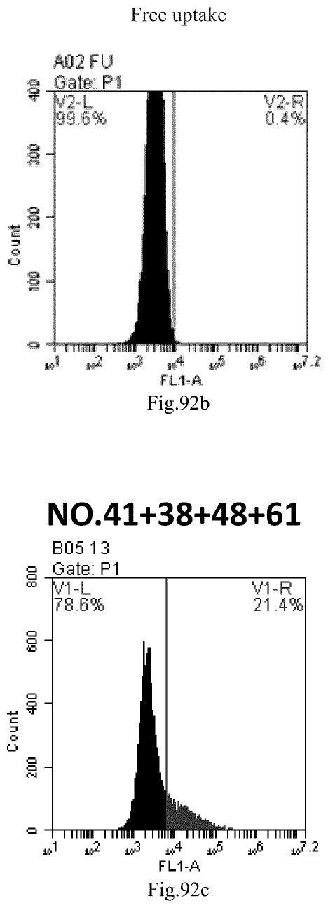

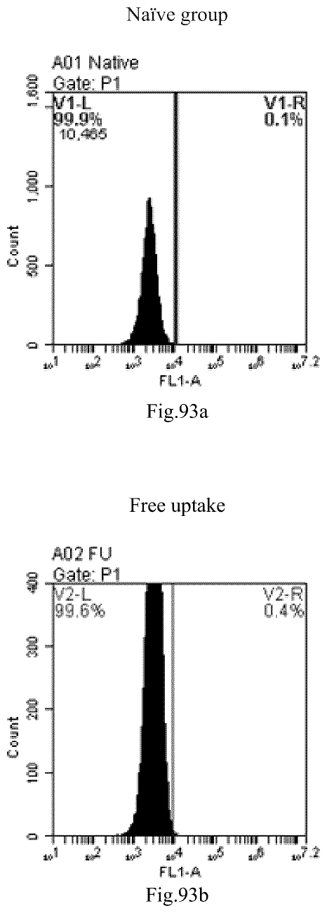

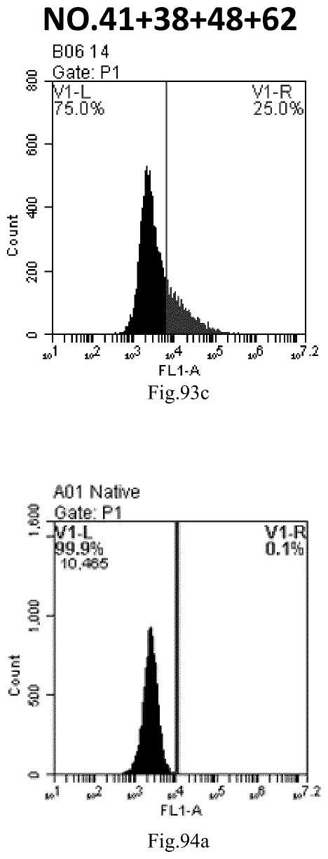

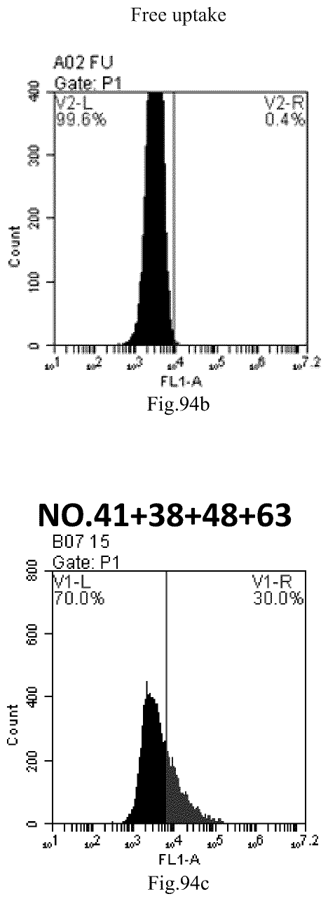

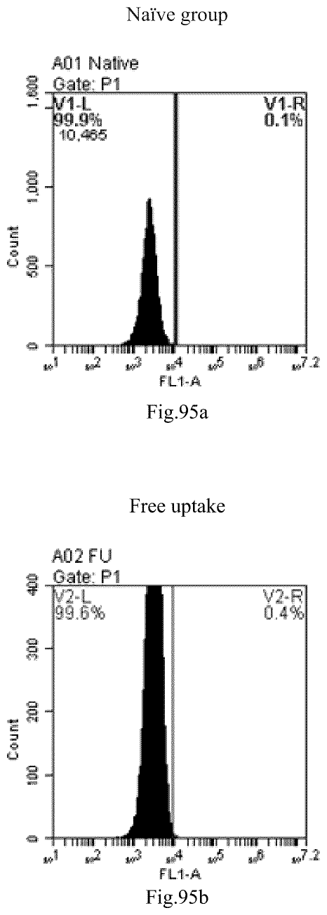

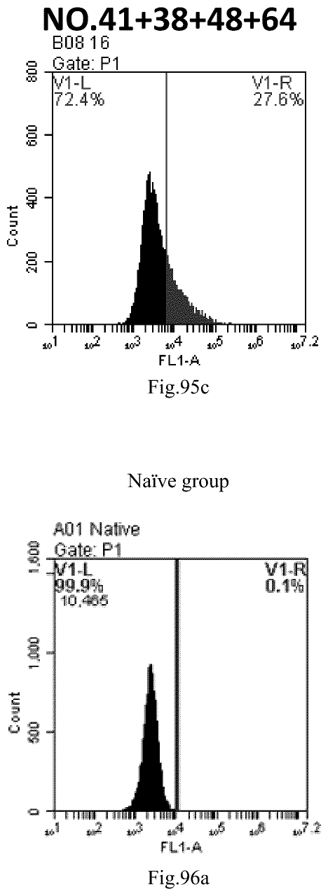

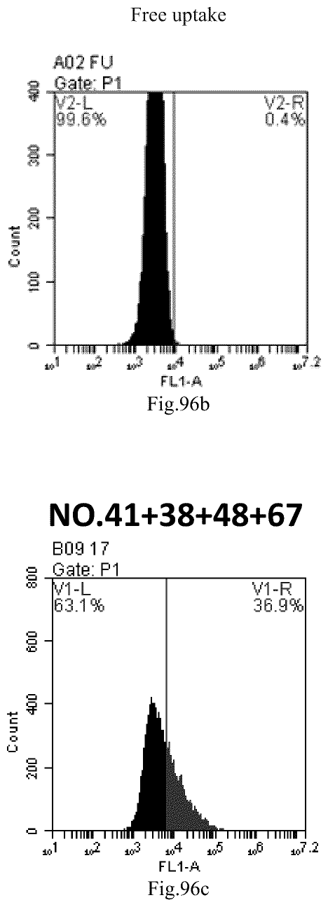

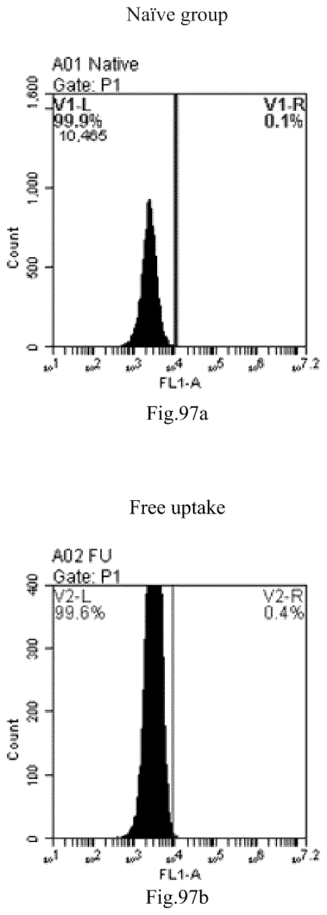

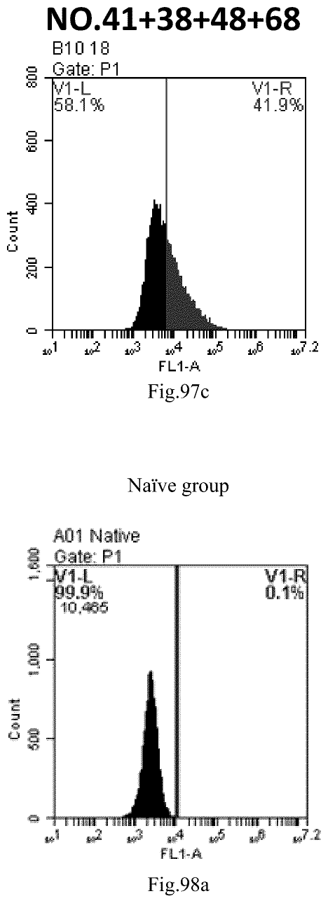

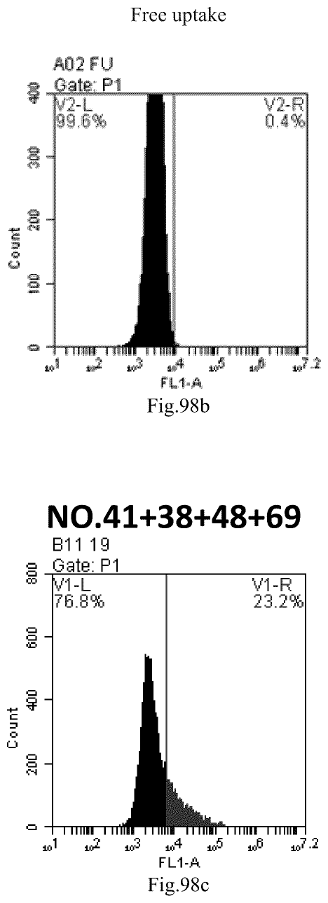

TABLE 4

| | | | | Lipid delivery group (different | | | | |
|---|---|---|---|---|---|---|---|---|
| | | | Free | concentrations of lipids) | | | | |
| | | Naïve | uptake | 3.75 μg/ | 12.5 μg/ | 37.5 μg/ | Delivery | Cell |
| FIGS. | Name of the lipid | group | group | mL | mL | mL | sequence | line |
| FIG. 1a-e | sphingosine | 0.00% | 0.20% | 1.60% | 1.50% | 1.40% | PGY- | 293T |
| | derivative | | | | | | ssRNA-6 | |
| | So-1 | | | | | | | |
| FIG. 2a-e | sphingosine | 0.00% | 0.20% | 1.40% | 2.10% | 3.40% | PGY- | 293T |
| | derivative | | | | | | ssRNA-6 | |
| | So-3 | | | | | | | |
| FIG. 3a-e | sphingosine | 0.00% | 0.20% | 1.70% | 2.20% | 0.00% | PGY- | 293T |
| | derivative | | | | | | ssRNA-6 | |
| | So-4 | | | | | | | |
| FIG. 4a-e | sphingosine | 0.00% | 0.20% | 2.80% | 1.60% | 2.40% | PGY- | 293T |
| | derivative | | | | | | ssRNA-6 | |
| | So-5 | | | | | | | |
| FIG. 5a-e | sphingosine | 0.00% | 0.20% | 1.20% | 0.20% | 4.30% | PGY- | 293T |
| | derivative | | | | | | ssRNA-6 | |
| | So-7 | | | | | | | |
| FIG. 6a-e | sphingosine | 0.00% | 0.20% | 3.60% | 4.50% | 1.90% | PGY- | 293T |
| | derivative | | | | | | ssRNA-6 | |
| | So-8 | | | | | | | |
| FIG. 7a-e | sphingosine | 0.00% | 0.10% | 3.30% | 2.60% | 2.70% | PGY- | 293T |
| | derivative | | | | | | ssRNA-6 | |
| | So-9 | | | | | | | |
| FIG. 8a-e | sphingosine | 0.00% | 0.10% | 2.10% | 3.40% | 2.50% | PGY- | 293T |
| | derivative | | | | | | ssRNA-6 | |
| | So-10 | | | | | | | |
| FIG. 9a-e | sphingosine | 0.00% | 0.10% | 3.60% | 7.40% | 12.70% | PGY- | 293T |
| | derivative | | | | | | ssRNA-6 | |
| | So-11 | | | | | | | |
| FIG. 10a-e | sphingosine | 0.00% | 0.10% | 5.60% | 2.30% | 1.60% | PGY- | 293T |
| | derivative | | | | | | ssRNA-6 | |
| | So-12 | | | | | | | |
| FIG. 11a-e | sphingosine | 0.00% | 0.10% | 3.40% | 3.20% | 5.20% | PGY- | 293T |
| | derivative | | | | | | ssRNA-6 | |
| | So-13 | | | | | | | |
| FIG. 12a-e | sphingosine | 0.00% | 0.10% | 4.40% | 3.30% | 4.30% | PGY- | 293T |
| | derivative | | | | | | ssRNA-6 | |
| | So-14 | | | | | | | |
| FIG. 13a-e | sphingosine | 0.00% | 0.10% | 4.70% | 1.90% | 2.40% | PGY- | 293T |
| | derivative | | | | | | ssRNA-6 | |
| | So-15 | | | | | | | |
| FIG. 14a-e | sphingosine | 0.10% | 0.20% | 0.20% | 3.80% | 0.50% | PGY- | 293T |
| | derivative | | | | | | ssRNA-6 | |
| | So-26 | | | | | | | |
| FIG. 15a-e | sphingosine | 0.10% | 0.20% | 0.20% | 0.30% | 6.90% | PGY- | 293T |
| | derivative | | | | | | ssRNA-6 | |
| | So-46 | | | | | | | |
| FIG. 16a-e | sphingosine | 0.10% | 0.20% | 0.50% | 1.70% | 7.40% | PGY- | 293T |
| | derivative | | | | | | ssRNA-6 | |
| | So-49 | | | | | | | |
| FIG. 17a-e | sphingosine | 0.10% | 0.20% | 0.20% | 0.30% | 6.70% | PGY- | 293T |
| | derivative | | | | | | ssRNA-6 | |
| | So-53 | | | | | | | |
| FIG. 18a-e | sphingosine | 0.00% | 0.60% | 7.10% | 7.10% | 6.20% | PGY- | 293T |
| | derivative | | | | | | ssRNA-6 | |
| | So-60 | | | | | | | |
| FIG. 19a-e | sphingosine | 0.00% | 0.60% | 7.40% | 8.10% | 7.00% | PGY- | 293T |
| | derivative | | | | | | ssRNA-6 | |
| | So-61 | | | | | | | |
| FIG. 20a-e | sphingosine | 0.00% | 0.60% | 6.60% | 6.50% | 7.10% | PGY- | 293T |
| | derivative | | | | | | ssRNA-6 | |
| | So-62 | | | | | | | |
| FIG. 21a-e | sphingosine | 0.00% | 0.60% | 5.60% | 7.70% | 6.50% | PGY- | 293T |
| | derivative | | | | | | ssRNA-6 | |
| | So-63 | | | | | | | |
| FIG. 22a-e | sphingosine | 0.00% | 0.60% | 7.40% | 6.20% | 8.70% | PGY- | 293T |
| | derivative | | | | | | ssRNA-6 | |
| | So-64 | | | | | | | |
| FIG. 23a-e | sphingosine | 0.00% | 0.60% | 7.60% | 7.50% | 6.80% | PGY- | 293T |
| | derivative | | | | | | ssRNA-6 | |
| | So-65 | | | | | | | |
| FIG. 24a-e | sphingosine | 0.00% | 0.60% | 7.10% | 18.10% | 29.50% | PGY- | 293T |
| | derivative | | | | | | ssRNA-6 | |
| | So-66 | | | | | | | |
| FIG. 25a-e | sphingosine | 0.00% | 0.60% | 6.30% | 5.30% | 2.80% | PGY- | 293T |
| | derivative | | | | | | ssRNA-6 | |
| | So-67 | | | | | | | |
| FIG. 26a-e | sphingosine | 0.00% | 0.60% | 4.80% | 3.80% | 3.90% | PGY- | 293T |
| | derivative | | | | | | ssRNA-6 | |
| | So-68 | | | | | | | |
| FIG. 27a-e | sphingosine | 0.00% | 0.60% | 4.60% | 3.80% | 3.30% | PGY- | 293T |
| | derivative | | | | | | ssRNA-6 | |
| | So-69 | | | | | | | |
| FIG. 28a-e | sphingosine | 0.00% | 0.60% | 4.70% | 10.10% | 11.40% | PGY- | 293T |
| | derivative | | | | | | ssRNA-6 | |
| | So-70 | | | | | | | |
| FIG. 29a-e | sphingosine | 0.00% | 0.60% | 3.60% | 6.20% | 8.80% | PGY- | 293T |
| | derivative | | | | | | ssRNA-6 | |
| | So-71 | | | | | | | |
| FIG. 30a-e | sphingosine | 0.00% | 0.60% | 2.40% | 3.40% | 14.10% | PGY- | 293T |
| | derivative | | | | | | ssRNA-6 | |
| | So-72 | | | | | | | |
| FIG. 31a-e | sphingosine | 0.00% | 0.60% | 3.20% | 4.60% | 9.50% | PGY- | 293T |
| | derivative | | | | | | ssRNA-6 | |
| | So-73 | | | | | | | |
| FIG. 32a-e | sphingosine | 0.00% | 0.60% | 4.00% | 3.10% | 6.60% | PGY- | 293T |
| | derivative | | | | | | ssRNA-6 | |
| | So-74 | | | | | | | |
| FIG. 33a-e | sphingosine | 0.00% | 0.60% | 3.00% | 4.20% | 11.40% | PGY- | 293T |
| | derivative | | | | | | ssRNA-6 | |
| | So-75 | | | | | | | |
| FIG. 34a-e | No. 41 + No. 38 + | 0.10% | 0.40% | | 6.30% | | PGY- | THP-1 |
| | sphingosine | | | | | | ssRNA-6 | |
| | derivative | | | | | | | |
| | So-1 | | | | | | | |
| FIG. 35a-c | No. 41 + No. 38 + | 0.10% | 0.40% | | 37.00% | | PGY- | THP-1 |
| | sphingosine | | | | | | ssRNA-6 | |
| | derivative | | | | | | | |
| | So-2 | | | | | | | |
| FIG. 36a-c | No. 41 + No. 38 + | 0.10% | 0.40% | | 29.00% | | PGY- | THP-1 |
| | sphingosine | | | | | | ssRNA-6 | |
| | derivative | | | | | | | |
| | So-3 | | | | | | | |
| FIG. 37a-c | No. 41 + No. 38 + | 0.10% | 0.40% | | 6.70% | | PGY- | THP-1 |
| | sphingosine | | | | | | ssRNA-6 | |
| | derivative | | | | | | | |
| | So-4 | | | | | | | |
| FIG. 38a-c | No. 41 + No. 38 + | 0.10% | 0.40% | | 20.40% | | PGY- | THP-1 |
| | sphingosine | | | | | | ssRNA-6 | |
| | derivative | | | | | | | |
| | So-5 | | | | | | | |
| FIG. 39a-c | No. 41 + No. 38 + | 0.10% | 0.40% | | 26.60% | | PGY- | THP-1 |
| | sphingosine | | | | | | ssRNA-6 | |
| | derivative | | | | | | | |
| | So-6 | | | | | | | |
| FIG. 40a-c | No. 41 + No. 38 + | 0.10% | 0.40% | | 19.90% | | PGY- | THP-1 |
| | sphingosine | | | | | | ssRNA-6 | |
| | derivative | | | | | | | |
| | So-7 | | | | | | | |
| FIG. 41a-c | No. 41 + No. 38 + | 0.10% | 0.40% | | 54.30% | | PGY- | THP-1 |
| | sphingosine | | | | | | ssRNA-6 | |
| | derivative | | | | | | | |
| | So-8 | | | | | | | |
| FIG. 42a-c | No. 41 + No. 38 + | 0.10% | 0.40% | | 21.20% | | PGY- | THP-1 |
| | sphingosine | | | | | | ssRNA-6 | |
| | derivative | | | | | | | |
| | So-9 | | | | | | | |
| FIG. 43a-c | No. 41 + No. 38 + | 0.10% | 0.40% | | 38.90% | | PGY- | THP-1 |
| | sphingosine | | | | | | ssRNA-6 | |
| | derivative | | | | | | | |
| | So-10 | | | | | | | |
| FIG. 44a-c | No. 41 + No. 38 + | 0.10% | 0.40% | | 7.30% | | PGY- | THP-1 |
| | sphingosine | | | | | | ssRNA-6 | |
| | derivative | | | | | | | |
| | So-11 | | | | | | | |
| FIG. 45a-c | No. 41 + No. 38 + | 0.10% | 0.40% | | 0.40% | | PGY- | THP-1 |
| | sphingosine | | | | | | ssRNA-6 | |
| | derivative | | | | | | | |
| | So-12 | | | | | | | |
| FIG. 46a-c | No. 41 + No. 38 + | 0.10% | 0.40% | | 26.20% | | PGY- | THP-1 |
| | sphingosine | | | | | | ssRNA-6 | |
| | derivative | | | | | | | |
| | So-13 | | | | | | | |
| FIG. 47a-c | No. 41 + No. 38 + | 0.10% | 0.40% | | 28.30% | | PGY- | THP-1 |
| | sphingosine | | | | | | ssRNA-6 | |
| | derivative | | | | | | | |
| | So-14 | | | | | | | |
| FIG. 48a-c | No. 41 + No. 38 + | 0.10% | 0.40% | | 31.80% | | PGY- | THP-1 |
| | sphingosine | | | | | | ssRNA-6 | |
| | derivative | | | | | | | |
| | So-15 | | | | | | | |
| FIG. 49a-c | No. 41 + No. 38 + | 0.10% | 0.40% | | 15.50% | | PGY- | THP-1 |
| | sphingosine | | | | | | ssRNA-6 | |
| | derivative | | | | | | | |
| | So-45 | | | | | | | |
| FIG. 50a-c | No. 41 + No. 38 + | 0.10% | 0.40% | | 31.40% | | PGY- | THP-1 |
| | sphingosine | | | | | | ssRNA-6 | |
| | derivative | | | | | | | |
| | So-46 | | | | | | | |
| FIG. 51a-c | No. 41 + No. 38 + | 0.10% | 0.40% | | 32.60% | | PGY- | THP-1 |
| | sphingosine | | | | | | ssRNA-6 | |
| | derivative | | | | | | | |
| | So-47 | | | | | | | |
| FIG. 52a-c | No. 41 + No. 38 + | 0.10% | 0.40% | | 37.40% | | PGY- | THP-1 |
| | sphingosine | | | | | | ssRNA-6 | |
| | derivative | | | | | | | |
| | So-48 | | | | | | | |
| FIG. 53a-c | No. 41 + No. 38 + | 0.10% | 0.40% | | 36.40% | | PGY- | THP-1 |
| | sphingosine | | | | | | ssRNA-6 | |
| | derivative | | | | | | | |
| | So-49 | | | | | | | |
| FIG. 54a-c | No. 41 + No. 38 + | 0.10% | 0.40% | | 34.40% | | PGY- | THP-1 |
| | sphingosine | | | | | | ssRNA-6 | |
| | derivative | | | | | | | |
| | So-50 | | | | | | | |
| FIG. 55a-c | No. 41 + No. 38 + | 0.10% | 0.40% | | 40.80% | | PGY- | THP-1 |
| | sphingosine | | | | | | ssRNA-6 | |
| | derivative | | | | | | | |
| | So-51 | | | | | | | |
| FIG. 56a-c | No. 41 + No. 38 + | 0.10% | 0.40% | | 22.60% | | PGY- | THP-1 |
| | sphingosine | | | | | | ssRNA-6 | |
| | derivative | | | | | | | |
| | So-52 | | | | | | | |
| FIG. 57a-c | No. 41 + No. 38 + | 0.10% | 0.40% | | 20.40% | | PGY- | THP-1 |
| | sphingosine | | | | | | ssRNA-6 | |
| | derivative | | | | | | | |
| | So-53 | | | | | | | |
| FIG. 58a-c | No. 41 + No. 38 + | 0.10% | 0.40% | | 30.70% | | PGY- | THP-1 |
| | sphingosine | | | | | | ssRNA-6 | |
| | derivative | | | | | | | |
| | So-54 | | | | | | | |
| FIG. 59a-c | No. 41 + No. 38 + | 0.10% | 0.40% | | 32.50% | | PGY- | THP-1 |
| | sphingosine | | | | | | ssRNA-6 | |
| | derivative | | | | | | | |
| | So-55 | | | | | | | |
| FIG. 60a-c | No. 41 + No. 38 + | 0.10% | 0.40% | | 41.00% | | PGY- | THP-1 |
| | sphingosine | | | | | | ssRNA-6 | |
| | derivative | | | | | | | |
| | So-56 | | | | | | | |
| FIG. 61a-c | No. 41 + No. 38 + | 0.10% | 0.40% | | 28.00% | | PGY- | THP-1 |
| | sphingosine | | | | | | ssRNA-6 | |
| | derivative | | | | | | | |
| | So-57 | | | | | | | |
| FIG. 62a-c | No. 41 + No. 38 + | 0.10% | 0.40% | | 17.70% | | PGY- | THP-1 |
| | sphingosine | | | | | | ssRNA-6 | |
| | derivative | | | | | | | |
| | So-58 | | | | | | | |
| FIG. 63a-c | No. 41 + No. 38 + | 0.10% | 0.40% | | 26.80% | | PGY- | THP-1 |
| | sphingosine | | | | | | ssRNA-6 | |
| | derivative | | | | | | | |
| | So-59 | | | | | | | |
| FIG. 64a-c | No. 41 + No. 38 + | 0.10% | 0.40% | | 43.80% | | PGY- | THP-1 |
| | sphingosine | | | | | | ssRNA-6 | |
| | derivative | | | | | | | |
| | So-60 | | | | | | | |
| FIG. 65a-c | No. 41 + No. 38 + | 0.10% | 0.40% | | 42.70% | | PGY- | THP-1 |
| | sphingosine | | | | | | ssRNA-6 | |
| | derivative | | | | | | | |
| | So-61 | | | | | | | |
| FIG. 66a-c | No. 41 + No. 38 + | 0.10% | 0.40% | | 39.70% | | PGY- | THP-1 |
| | sphingosine | | | | | | ssRNA-6 | |
| | derivative | | | | | | | |
| | So-62 | | | | | | | |
| FIG. 67a-c | No. 41 + No. 38 + | 0.10% | 0.40% | | 16.80% | | PGY- | THP-1 |
| | sphingosine | | | | | | ssRNA-6 | |
| | derivative | | | | | | | |
| | So-63 | | | | | | | |
| FIG. 68a-c | No. 41 + No. 38 + | 0.10% | 0.40% | | 30.70% | | PGY- | THP-1 |
| | sphingosine | | | | | | ssRNA-6 | |
| | derivative | | | | | | | |
| | So-64 | | | | | | | |
| FIG. 69a-c | No. 41 + No. 38 + | 0.10% | 0.40% | | 40.90% | | PGY- | THP-1 |
| | sphingosine | | | | | | ssRNA-6 | |
| | derivative | | | | | | | |
| | So-65 | | | | | | | |
| FIG. 70a-c | No. 41 + No. 38 + | 0.10% | 0.40% | | 38.40% | | PGY- | THP-1 |
| | sphingosine | | | | | | ssRNA-6 | |
| | derivative | | | | | | | |
| | So-66 | | | | | | | |
| FIG. 71a-c | No. 41 + No. 38 + | 0.10% | 0.40% | | 23.50% | | PGY- | THP-1 |
| | sphingosine | | | | | | ssRNA-6 | |
| | derivative | | | | | | | |
| | So-67 | | | | | | | |
| FIG. 72a-c | No. 41 + No. 38 + | 0.10% | 0.40% | | 26.90% | | PGY- | THP-1 |
| | sphingosine | | | | | | ssRNA-6 | |
| | derivative | | | | | | | |
| | So-68 | | | | | | | |
| FIG. 73a-c | No. 41 + No. 38 + | 0.10% | 0.40% | | 34.60% | | PGY- | THP-1 |
| | sphingosine | | | | | | ssRNA-6 | |
| | derivative | | | | | | | |
| | So-69 | | | | | | | |
| FIG. 74a-c | No. 41 + No. 38 + | 0.10% | 0.40% | | 12.20% | | PGY- | THP-1 |
| | sphingosine | | | | | | ssRNA-6 | |
| | derivative | | | | | | | |
| | So-70 | | | | | | | |
| FIG. 75a-c | No. 41 + No. 38 + | 0.10% | 0.40% | | 14.10% | | PGY- | THP-1 |
| | sphingosine | | | | | | ssRNA-6 | |
| | derivative | | | | | | | |
| | So-71 | | | | | | | |
| FIG. 76a-c | No. 41 + No. 38 + | 0.10% | 0.40% | | 44.40% | | PGY- | THP-1 |
| | sphingosine | | | | | | ssRNA-6 | |
| | derivative | | | | | | | |
| | So-72 | | | | | | | |
| FIG. 77a-c | No. 41 + No. 38 + | 0.10% | 0.40% | | 7.30% | | PGY- | THP-1 |
| | sphingosine | | | | | | ssRNA-6 | |
| | derivative | | | | | | | |
| | So-73 | | | | | | | |
| FIG. 78a-c | No. 41 + No. 38 + | 0.10% | 0.40% | | 30.70% | | PGY- | THP-1 |
| | sphingosine | | | | | | ssRNA-6 | |
| | derivative | | | | | | | |
| | So-74 | | | | | | | |
| FIG. 79a-c | No. 41 + No. 38 + | 0.10% | 0.40% | | 17.70% | | PGY- | THP-1 |
| | sphingosine | | | | | | ssRNA-6 | |
| | derivative | | | | | | | |
| | So-75 | | | | | | | |
| FIG. 80a-c | No. 41 + No. 38 + | 0.10% | 0.40% | | 18.40% | | PGY- | THP-1 |
| | No. 48 + sphingosine | | | | | | ssRNA-6 | |
| | derivative | | | | | | | |
| | So-42 | | | | | | | |
| FIG. 81a-c | No. 41 + No. 38 + | 0.10% | 0.40% | | 19.40% | | PGY- | THP-1 |
| | No. 48 + sphingosine | | | | | | ssRNA-6 | |
| | derivative | | | | | | | |
| | So-43 | | | | | | | |
| FIG. 82a-c | No. 41 + No. 38 + | 0.10% | 0.40% | | 29.70% | | PGY- | THP-1 |
| | No. 48 + sphingosine | | | | | | ssRNA-6 | |
| | derivative | | | | | | | |
| | So-44 | | | | | | | |
| FIG. 83a-c | No. 41 + No. 38 + | 0.10% | 0.40% | | 33.20% | | PGY- | THP-1 |
| | No. 48 + sphingosine | | | | | | ssRNA-6 | |
| | derivative | | | | | | | |
| | So-45 | | | | | | | |
| FIG. 84a-c | No. 41 + No. 38 + | 0.10% | 0.40% | | 29.00% | | PGY- | THP-1 |
| | No. 48 + sphingosine | | | | | | ssRNA-6 | |
| | derivative | | | | | | | |
| | So-46 | | | | | | | |
| FIG. 85a-c | No. 41 + No. 38 + | 0.10% | 0.40% | | 34.60% | | PGY- | THP-1 |
| | No. 48 + sphingosine | | | | | | ssRNA-6 | |
| | derivative | | | | | | | |
| | So-47 | | | | | | | |
| FIG. 86a-c | No. 41 + No. 38 + | 0.10% | 0.40% | | 31.20% | | PGY- | THP-1 |
| | No. 48 + sphingosine | | | | | | ssRNA-6 | |
| | derivative | | | | | | | |
| | So-52 | | | | | | | |
| FIG. 87a-c | No. 41 + No. 38 + | 0.10% | 0.40% | | 39.30% | | PGY- | THP-1 |
| | No. 48 + sphingosine | | | | | | ssRNA-6 | |
| | derivative | | | | | | | |
| | So-56 | | | | | | | |
| FIG. 88a-c | No. 41 + No. 38 + | 0.10% | 0.40% | | 32.70% | | PGY- | THP-1 |
| | No. 48 + sphingosine | | | | | | ssRNA-6 | |
| | derivative | | | | | | | |
| | So-57 | | | | | | | |
| FIG. 89a-c | No. 41 + No. 38 + | 0.10% | 0.40% | | 5.00% | | PGY- | THP-1 |
| | No. 48 + sphingosine | | | | | | ssRNA-6 | |
| | derivative | | | | | | | |
| | So-58 | | | | | | | |
| FIG. 90a-c | No. 41 + No. 38 + | 0.10% | 0.40% | | 21.60% | | PGY- | THP-1 |
| | No. 48 + sphingosine | | | | | | ssRNA-6 | |
| | derivative | | | | | | | |
| | So-59 | | | | | | | |
| FIG. 91a-c | No. 41 + No. 38 + | 0.10% | 0.40% | | 22.30% | | PGY- | THP-1 |
| | No. 48 + sphingosine | | | | | | ssRNA-6 | |
| | derivative | | | | | | | |
| | So-60 | | | | | | | |
| FIG. 92a-c | No. 41 + No. 38 + | 0.10% | 0.40% | | 21.40% | | PGY- | THP-1 |
| | No. 48 + sphingosine | | | | | | ssRNA-6 | |
| | derivative | | | | | | | |
| | So-61 | | | | | | | |
| FIG. 93a-c | No. 41 + No. 38 + | 0.10% | 0.40% | | 25.00% | | PGY- | THP-1 |
| | No. 48 + sphingosine | | | | | | ssRNA-6 | |
| | derivative | | | | | | | |
| | So-62 | | | | | | | |
| FIG. 94a-c | No. 41 + No. 38 + | 0.10% | 0.40% | | 30.00% | | PGY- | THP-1 |
| | No. 48 + sphingosine | | | | | | ssRNA-6 | |
| | derivative | | | | | | | |
| | So-63 | | | | | | | |
| FIG. 95a-c | No. 41 + No. 38 + | 0.10% | 0.40% | | 27.60% | | PGY- | THP-1 |
| | No. 48 + sphingosine | | | | | | ssRNA-6 | |
| | derivative | | | | | | | |
| | So-64 | | | | | | | |
| FIG. 96a-c | No. 41 + No. 38 + | 0.10% | 0.40% | | 36.90% | | PGY- | THP-1 |
| | No. 48 + sphingosine | | | | | | ssRNA-6 | |
| | derivative | | | | | | | |
| | So-67 | | | | | | | |
| FIG. 97a-c | No. 41 + No. 38 + | 0.10% | 0.40% | | 41.90% | | PGY- | THP-1 |
| | No. 48 + sphingosine | | | | | | ssRNA-6 | |
| | derivative | | | | | | | |
| | So-68 | | | | | | | |
| FIG. 98a-c | No. 41 + No. 38 + | 0.10% | 0.40% | | 23.20% | | PGY- | THP-1 |
| | No. 48 + sphingosine | | | | | | ssRNA-6 | |
| | derivative | | | | | | | |
| | So-69 | | | | | | | |
| FIG. 99a-c | No. 41 + No. 38 + | 0.10% | 0.40% | | 12.80% | | PGY- | THP-1 |
| | No. 48 + sphingosine | | | | | | ssRNA-6 | |
| | derivative | | | | | | | |
| | So-70 | | | | | | | |

### EXPLANATION

1. Compared with the negative control group, the fluorescence shift (shifted to the right, marked with red area) was increased in the experimental group, which indicated that more fluorescently labeled RNA had been taken up by cells, that is, larger amount of RNA was delivered into the cell by lipid, reflecting the higher delivery efficiency;
2. The delivery efficiency varied with lipid concentration, which indicated that the delivery of RNA was dependent on the lipid concentration.

1. A method for delivering a nucleic acid to a cell or a subject, comprising:
mixing a lipid composition with the nucleic acid to obtain a lipid-nucleic acid mixture, and
delivering the lipid-nucleic acid mixture so that the nucleic acid is delivered to the cell or the subject,
wherein the lipid composition comprises one or more compounds of Formula (I), a stereoisomer or a pharmaceutical acceptable salt thereof: 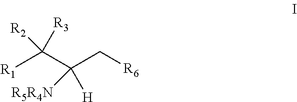
wherein:
R1 is selected from C₁₋₂₀alkyl or C₂₋₂₀alkenyl, which is optionally substituted by one to three hydroxyl groups;
R2 is hydrogen and R3 is hydroxyl; or
R2 and R3 together form oxo (═O);
R4 and R5 are independently selected from the group consisting of hydrogen, C₁₋₂₀alkyl, C₁₋₃₀alkylacyl and C₁₋₃₀alkenylacyl, said C₁₋₃₀alkylacyl and C₁₋₃₀alkenylacyl are optionally substituted by one to three groups selected from biotin acyl or hydroxyl;
or —NR4R5 group is a quaternary ammonium cation;
R6 is selected from the group consisting of hydrogen, hydroxyl, phosphate ester group, —O-glycosyl, Ganglioside, and aminoethoxyphosphonate ester group (NH₂—CH₂—CH₂—O—P(O)OH—).

2. The method of claim 1, wherein:
R1 is selected from C₁₄₋₂₀alkyl or C₁₄₋₂₀alkenyl containing one double bond;
R4 and R5 are independently selected from the group consisting of hydrogen, C₁₋₆alkyl, C₆₋₃₀alkylacyl and C₆₋₃₀alkenylacyl, said C₆₋₃₀alkylacyl and the C₆₋₃₀alkenylacyl are optionally substituted at the terminal by one group selected from biotin acyl or hydroxyl, or optionally substituted at the α-carbon of the acyl by a hydroxyl.

3. The method of claim 1, wherein
(i) R4 and R5 are both methyl;
(ii) at least one of R4 and R5 is hydrogen and the other is straight-chain C₆₋₃₀alkylacyl, straight-chain C₆₋₃₀alkenylacyl or straight-chain C₇₋₁₄alkyl; or
(iii) R4 and R5 are independently selected from C₁₋₆alkyl.

4. The method of claim 1, wherein R1 is selected from a straight-chain C₁₄₋₂₀alkyl or a straight-chain C₁₄₋₂₀alkenyl containing one double bond.

5. The method of claim 1, wherein the glycosyl is glucosyl, lactosyl, or galactosyl.

6. The method of claim 1, wherein the —NR4R5 group is —NH₃⁺ or N(CH₃)₃⁺.

7. The method of claim 1, wherein the glycosyl is 1-β-D-glucosyl;
R2 is hydrogen, and R3 is hydroxyl;
R1 is selected from C₁₄₋₂₀alkenyl containing one double bond, and the alkenyl is immediately adjacent to the carbon atom to which R2 and R3 are attached.

8. The method of claim 1, wherein the compound has the following Formula (Ia): 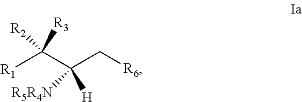
wherein R1, R2, R3, R4, R5 and R6 are as defined in claim 1.

9. The method of claim 1, wherein the lipid composition comprises one or more of the following lipids:

| No. | Structure |
|---|---|
| So-1 | 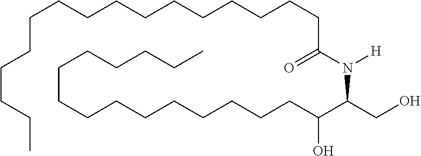 [SMILES: CCCCCCCCCCCCCCCCC(=O)N[C@@H](CO)C(O)CCCCCCCCCCCCCCC] |
| So-2 | 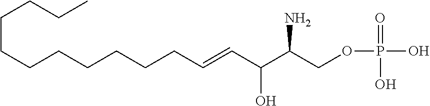 [SMILES: CCCCCCCCCCC/C=C/C(O)[C@@H](N)COP(=O)(O)O] |
| So-3 | 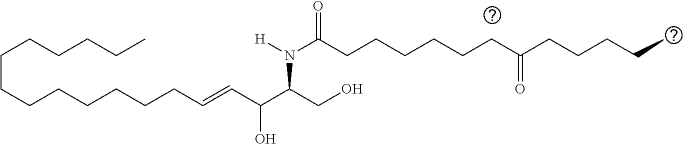 |
| So-4 | 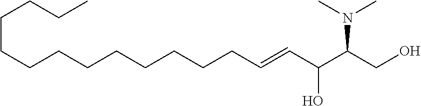 [SMILES: CCCCCCCCCCCCC/C=C/C(O)[C@H](CO)N(C)C] |
| So-5 | 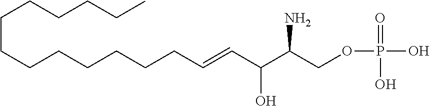 [SMILES: CCCCCCCCCCCCC/C=C/C(O)[C@@H](N)COP(=O)(O)O] |
| So-6 | 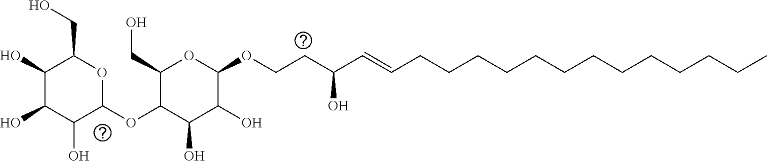 [SMILES: CCCCCCCCCCCCC/C=C/[C@@H](O)CCO[C@@H]1O[C@H](CO)C(OC2O[C@H](CO)[C@H](O)[C@H](O)C2O)[C@H](O)C1O] |
| So-7 | 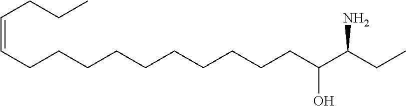 [SMILES: CCC/C=C\CCCCCCCCCCC(O)[C@@H](N)CC] |
| So-8 | 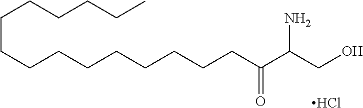 [SMILES: CCCCCCCCCCCCCCCC(=O)C(N)CO ; Cl] |
| So-9 | 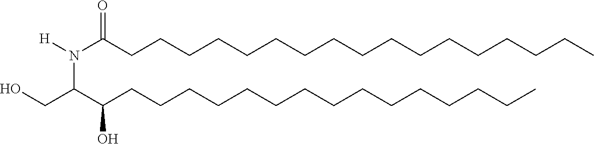 [SMILES: CCCCCCCCCCCCCCCCCC(=O)NC(CO)[C@H](O)CCCCCCCCCCCCCCC] |
| So-10 | 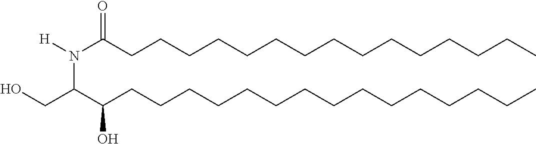 [SMILES: CCCCCCCCCCCCCCCC(=O)NC(CO)[C@H](O)CCCCCCCCCCCCCCC] |
| So-11 | 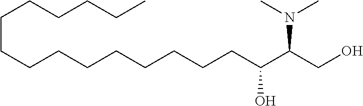 [SMILES: CCCCCCCCCCCCCCC[C@@H](O)[C@H](CO)N(C)C] |
| So-12 | 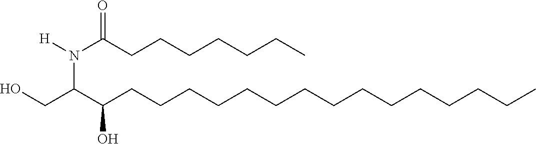 [SMILES: CCCCCCCCCCCCCCC[C@@H](O)C(CO)NC(=O)CCCCCCC] |
| So-13 | 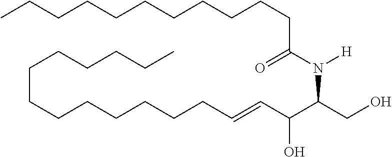 [SMILES: CCCCCCCCCCCCC/C=C/C(O)[C@H](CO)NC(=O)CCCCCCCCCCC] |
| So-14 | 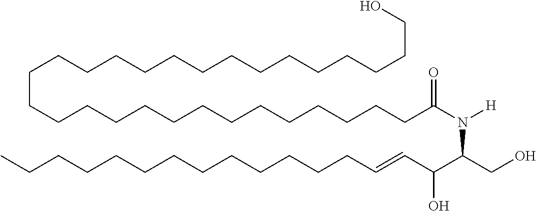 [SMILES: CCCCCCCCCCCCC/C=C/C(O)[C@H](CO)NC(=O)CCCCCCCCCCCCCCCCCCCCCCCCCCCCCO] |
| So-15 | 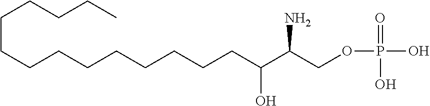 [SMILES: CCCCCCCCCCCCCCC(O)[C@@H](N)COP(=O)(O)O] |
| So-16 | 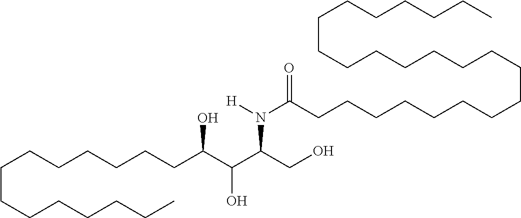 [SMILES: CCCCCCCCCCCCCCCCCCCCCCCC(=O)N[C@@H](CO)C(O)[C@H](O)CCCCCCCCCCCCCC] |
| So-17 | 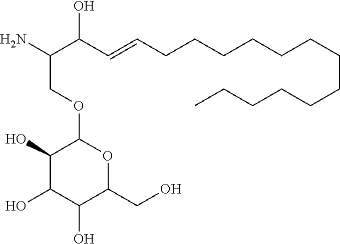 [SMILES: CCCCCCCCCCCCC/C=C/C(O)C(N)COC1OC(CO)C(O)C(O)[C@H]1O] |
| So-18 | 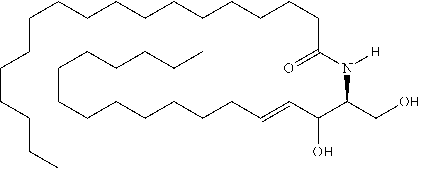 [SMILES: CCCCCCCCCCCCC/C=C/C(O)[C@H](CO)NC(=O)CCCCCCCCCCCCCCCCC] |
| So-19 | 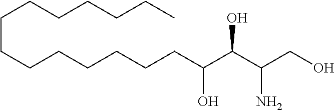 [SMILES: CCCCCCCCCCCCCCC(O)[C@@H](O)C(N)CO] |
| So-20 | 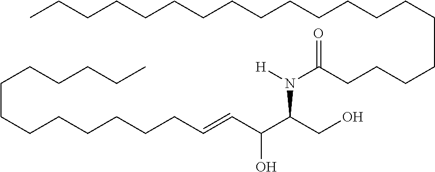 [SMILES: CCCCCCCCCCCCC/C=C/C(O)[C@H](CO)NC(=O)CCCCCCCCCCCCCCCCCCC] |
| So-21 | 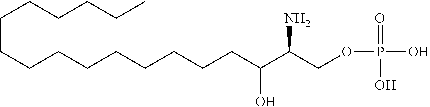 [SMILES: CCCCCCCCCCCCCCCC(O)[C@@H](N)COP(=O)(O)O] |
| So-23 | 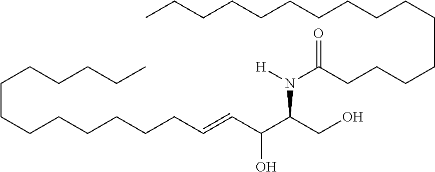 [SMILES: CCCCCCCCCCCCC/C=C/C(O)[C@H](CO)NC(=O)CCCCCCCCCCCCCCC] |
| So-25 | 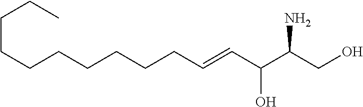 [SMILES: CCCCCCCCCC/C=C/C(O)[C@@H](N)CO] |
| So-26 | 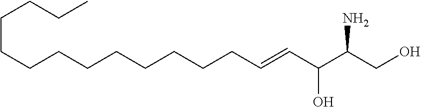 [SMILES: CCCCCCCCCCCCC/C=C/C(O)[C@@H](N)CO] |
| So-27 | 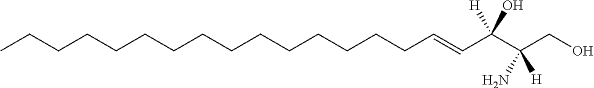 [SMILES: CCCCCCCCCCCCCCC/C=C/[C@@H](O)[C@@H](N)CO] |
| So-29 | 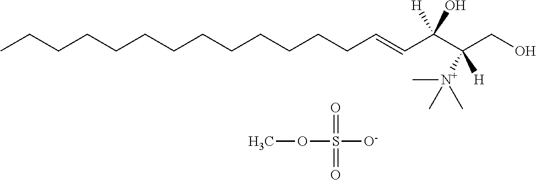 |
| So-30 | 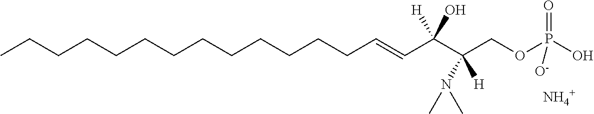 [SMILES: CCCCCCCCCCCCC/C=C/[C@@H](O)[C@H](COP(=O)([O-])O)N(C)C ; [NH4+]] |
| So-31 | 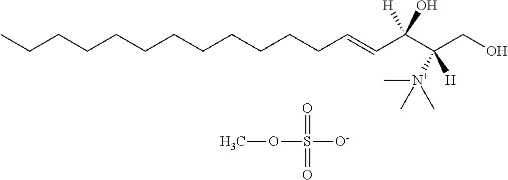 |
| So-32 | 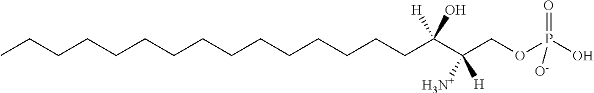 [SMILES: CCCCCCCCCCCCCCC[C@@H](O)[C@@H]([NH3+])COP(=O)([O-])O] |
| So-33 | 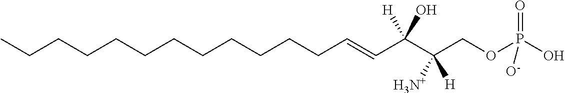 [SMILES: CCCCCCCCCCCC/C=C/[C@@H](O)[C@@H]([NH3+])COP(=O)([O-])O] |
| So-34 | 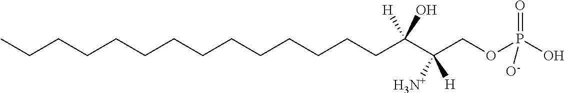 [SMILES: CCCCCCCCCCCCCC[C@@H](O)[C@@H]([NH3+])COP(=O)([O-])O] |
| So-35 | 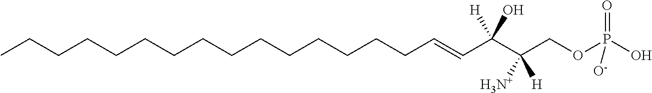 [SMILES: CCCCCCCCCCCCCCC/C=C/[C@@H](O)[C@@H]([NH3+])COP(=O)([O-])O] |
| So-36 | 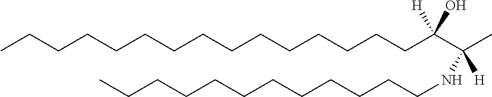 [SMILES: CCCCCCCCCCCCCCC[C@@H](O)[C@H](C)NCCCCCCCCCCCC] |
| So-37 | 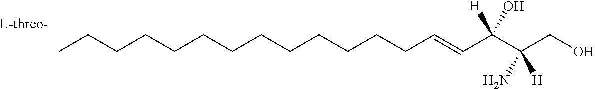 [SMILES: CCCCCCCCCCCCC/C=C/[C@H](O)[C@@H](N)CO] |
| So-38 | 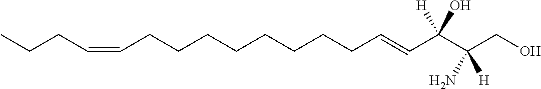 [SMILES: CCC/C=C\CCCCCCCC/C=C/[C@@H](O)[C@@H](N)CO] |
| So-40 | 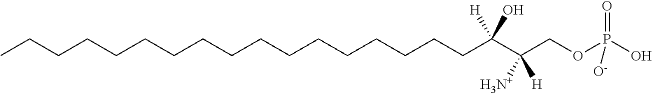 [SMILES: CCCCCCCCCCCCCCCCC[C@@H](O)[C@@H]([NH3+])COP(=O)([O-])O] |
| So-41 | 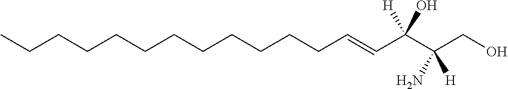 [SMILES: CCCCCCCCCCCC/C=C/[C@@H](O)[C@@H](N)CO] |
| So-42 | 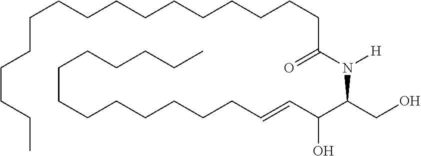 [SMILES: CCCCCCCCCCCCC/C=C/C(O)[C@H](CO)NC(=O)CCCCCCCCCCCCCCCC] |
| So-43 | 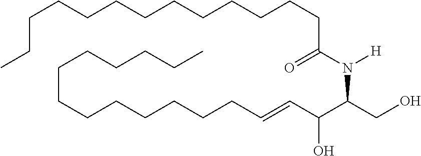 [SMILES: CCCCCCCCCCCCC/C=C/C(O)[C@H](CO)NC(=O)CCCCCCCCCCCCC] |
| So-44 | 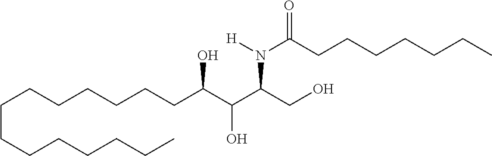 [SMILES: CCCCCCCCCCCCCC[C@@H](O)C(O)[C@H](CO)NC(=O)CCCCCCC] |
| So-45 | 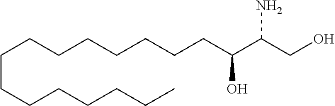 [SMILES: CCCCCCCCCCCCCCC[C@H](O)[C@H](N)CO] |
| So-46 | 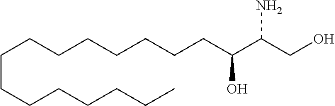 [SMILES: CCCCCCCCCCCCCCC[C@H](O)[C@H](N)CO] |
| So-47 | 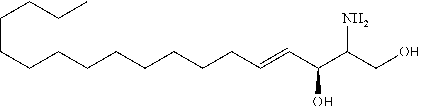 [SMILES: CCCCCCCCCCCCC/C=C/[C@H](O)C(N)CO] |
| So-48 | 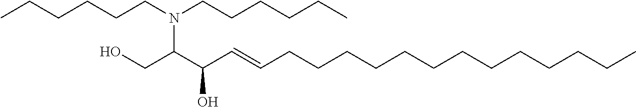 [SMILES: CCCCCCCCCCCCC/C=C/[C@@H](O)C(CO)N(CCCCCC)CCCCCC] |
| So-49 | 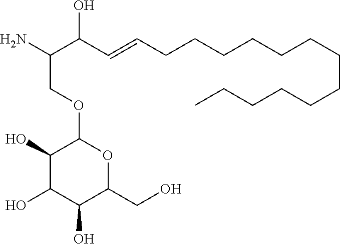 [SMILES: CCCCCCCCCCCCC/C=C/C(O)C(N)COC1OC(CO)[C@@H](O)C(O)[C@H]1O] |
| So-50 | 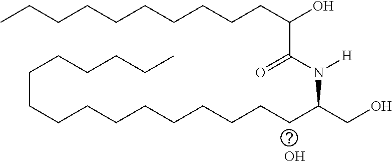 |
| So-51 | 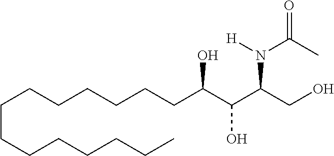 [SMILES: CCCCCCCCCCCCCC[C@@H](O)[C@@H](O)[C@H](CO)NC(C)=O] |
| So-52 | 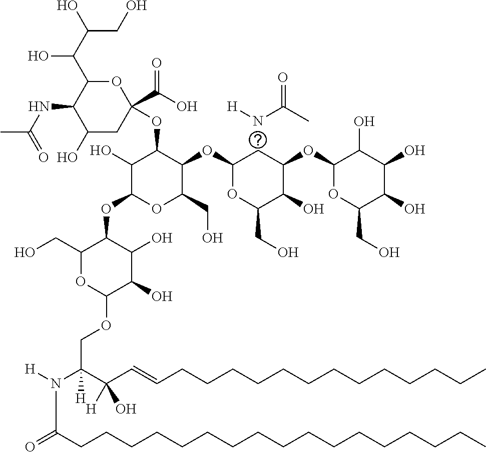 |
| So-53 | 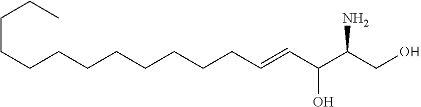 [SMILES: CCCCCCCCCCCC/C=C/C(O)[C@@H](N)CO] |
| So-54 | 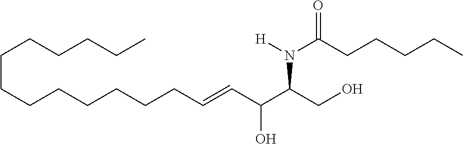 [SMILES: CCCCCCCCCCCCC/C=C/C(O)[C@H](CO)NC(=O)CCCCC] |
| So-55 | 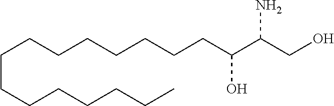 [SMILES: CCCCCCCCCCCCCCC[C@@H](O)[C@H](N)CO] |
| So-56 | 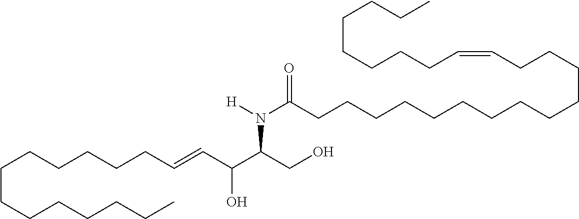 [SMILES: CCCCCCCC/C=C\CCCCCCCCCCCCCC(=O)N[C@@H](CO)C(O)/C=C/CCCCCCCCCCCCC] |
| So-57 | 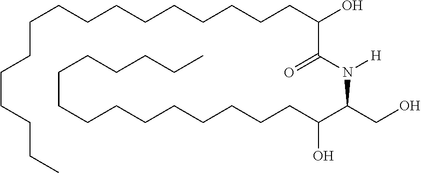 [SMILES: CCCCCCCCCCCCCCCCC(O)C(=O)N[C@@H](CO)C(O)CCCCCCCCCCCCCCC] |
| So-58 | 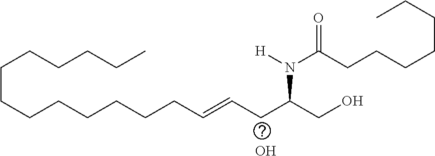 |
| So-59 | 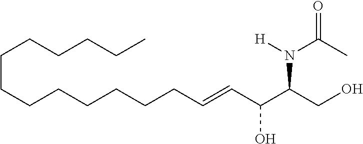 [SMILES: CCCCCCCCCCCCC/C=C/[C@@H](O)[C@H](CO)NC(C)=O] |
| So-60 | 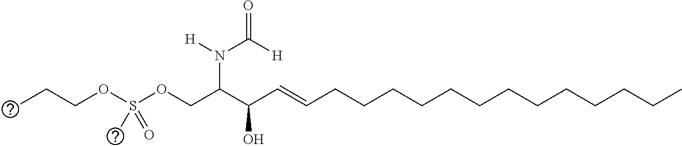 |
| So-61 | 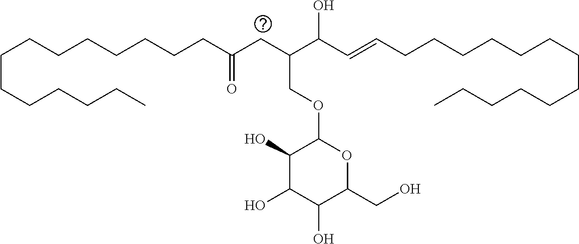 [SMILES: CCCCCCCCCCCCC/C=C/C(O)C(COC1OC(CO)C(O)C(O)[C@H]1O)CC(=O)CCCCCCCCCCCCCC] |
| So-62 | 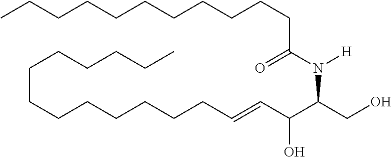 [SMILES: CCCCCCCCCCCCC/C=C/C(O)[C@H](CO)NC(=O)CCCCCCCCCCC] |
| So-63 | 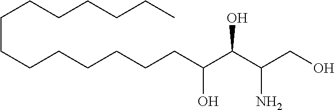 [SMILES: CCCCCCCCCCCCCCC(O)[C@@H](O)C(N)CO] |
| So-64 | 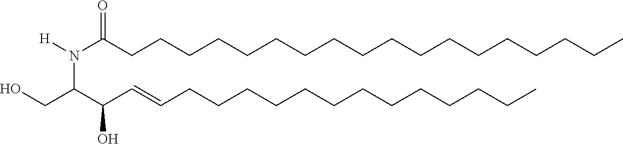 [SMILES: CCCCCCCCCCCCC/C=C/[C@@H](O)C(CO)NC(=O)CCCCCCCCCCCCCCCCCC] |
| So-65 | 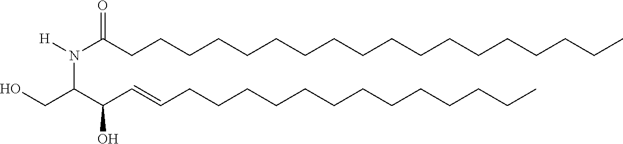 [SMILES: CCCCCCCCCCCCC/C=C/[C@@H](O)C(CO)NC(=O)CCCCCCCCCCCCCCCCCC] |
| So-66 | 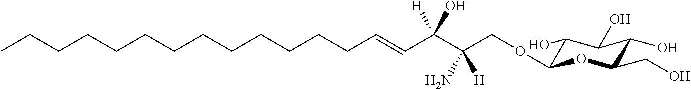 |
| So-67 | 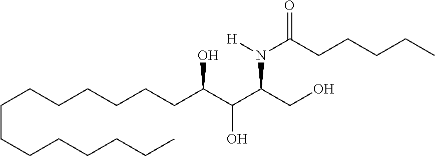 [SMILES: CCCCCCCCCCCCCC[C@@H](O)C(O)[C@H](CO)NC(=O)CCCCC] |
| So-68 | 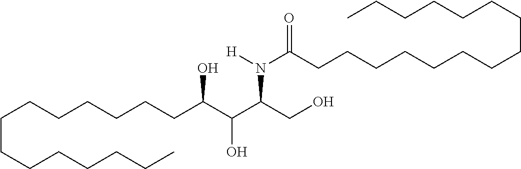 [SMILES: CCCCCCCCCCCCCCCC(=O)N[C@@H](CO)C(O)[C@H](O)CCCCCCCCCCCCCC] |
| So-69 | 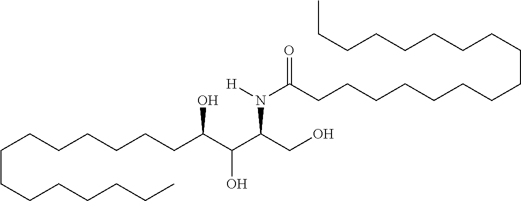 [SMILES: CCCCCCCCCCCCCCCCCC(=O)N[C@@H](CO)C(O)[C@H](O)CCCCCCCCCCCCCC] |
| So-70 | 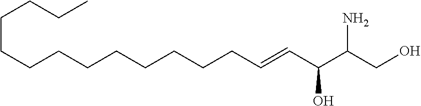 [SMILES: CCCCCCCCCCCCC/C=C/[C@H](O)C(N)CO] |
| So-71 | 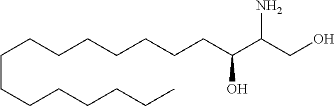 [SMILES: CCCCCCCCCCCCCCC[C@H](O)C(N)CO] |
| So-72 | 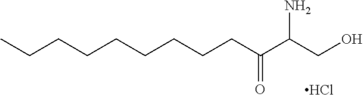 |
| So-73 | 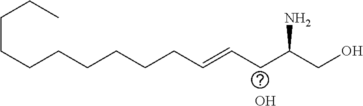 |
| So-74 | 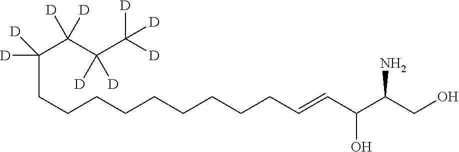 [SMILES: [2H]C([2H])([2H])C([2H])([2H])C([2H])([2H])C([2H])([2H])CCCCCCCCC/C=C/C(O)[C@@H](N)CO] |
| So-75 | 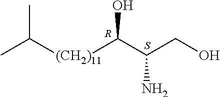 |
| No.41 | 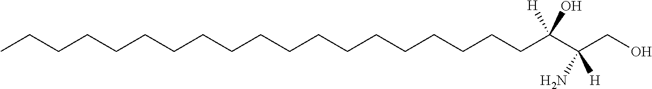 [SMILES: CCCCCCCCCCCCCCCCCCC[C@@H](O)[C@@H](N)CO] |
| No.38 | 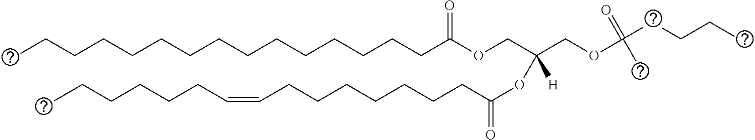 |
| No.48 | 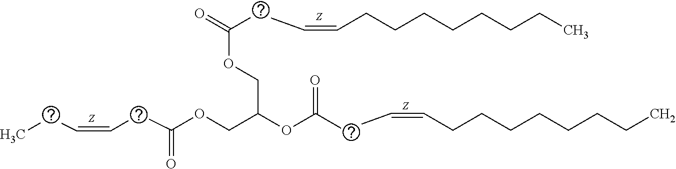 [SMILES: CC/C=C\CC(=O)OCC(COC(=O)C/C=C\CCCCCCCC)OC(=O)C/C=C\CCCCCCCC] |

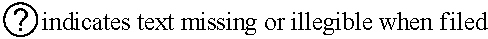

10. The method of claim 9, wherein the combination is a combination comprising any one or more of No. 41, No. 38, and No. 48, a combination comprising any one or more of No. 41, No. 38, No. 48 and any one or more of other compounds selected from claim 9, or a combination comprising the following: No. 41+No. 38+sphingosine derivative So-1; No. 41+No. 38+sphingosine derivative So-2; No. 41+No. 38+sphingosine derivative So-3; No. 41+No. 38+sphingosine derivative So-4; No. 41+No. 38+sphingosine derivative So-5; No. 41+No. 38+sphingosine derivative So-6; No. 41+No. 38+sphingosine derivative So-7; No. 41+No. 38+sphingosine derivative So-8; No. 41+No. 38+sphingosine derivative So-9; No. 41+No. 38+sphingosine derivative So-10; No. 41+No. 38+sphingosine derivative So-11; No. 41+No. 38+sphingosine derivative So-12; No. 41+No. 38+sphingosine derivative So-13; No. 41+No. 38+sphingosine derivative So-14; No. 41+No. 38+sphingosine derivative So-15; No. 41+No. 38+sphingosine derivative So-45; No. 41+No. 38+sphingosine derivative So-46; No. 41+No. 38+sphingosine derivative So-47; No. 41+No. 38+sphingosine derivative So-48; No. 41+No. 38+sphingosine derivative So-49; No. 41+No. 38+sphingosine derivative So-50; No. 41+No. 38+sphingosine derivative So-51; No. 41+No. 38+sphingosine derivative So-52; No. 41+No. 38+sphingosine derivative So-53; No. 41+No. 38+sphingosine derivative So-54; No. 41+No. 38+sphingosine derivative So-55; No. 41+No. 38+sphingosine derivative So-56; No. 41+No. 38+sphingosine derivative So-57; No. 41+No. 38+sphingosine derivative So-58; No. 41+No. 38+sphingosine derivative So-59; No. 41+No. 38+sphingosine derivative So-60; No. 41+No. 38+sphingosine derivative So-61; No. 41+No. 38+sphingosine derivative So-62; No. 41+No. 38+sphingosine derivative So-63; No. 41+No. 38+sphingosine derivative So-64; No. 41+No. 38+sphingosine derivative So-65; No. 41+No. 38+sphingosine derivative So-66; No. 41+No. 38+sphingosine derivative So-67; No. 41+No. 38+sphingosine derivative So-68; No. 41+No. 38+sphingosine derivative So-69; No. 41+No. 38+sphingosine derivative So-70; No. 41+No. 38+sphingosine derivative So-71; No. 41+No. 38+sphingosine derivative So-72; No. 41+No. 38+sphingosine derivative So-73; No. 41+No. 38+sphingosine derivative So-74; No. 41+No. 38+sphingosine derivative So-75; No. 41+No. 38+No. 48+sphingosine derivative So-42; No. 41+No. 38+No. 48+sphingosine derivative So-43; No. 41+No. 38+No. 48+sphingosine derivative So-44; No. 41+No. 38+No. 48+sphingosine derivative So-45; No. 41+No. 38+No. 48+sphingosine derivative So-46; No. 41+No. 38+No. 48+sphingosine derivative So-47; No. 41+No. 38+No. 48+sphingosine derivative So-52; No. 41+No. 38+No. 48+sphingosine derivative So-56; No. 41+No. 38+No. 48+sphingosine derivative So-57; No. 41+No. 38+No. 48+sphingosine derivative So-58; No. 41+No. 38+No. 48+sphingosine derivative So-59; No. 41+No. 38+No. 48+sphingosine derivative So-60; No. 41+No. 38+No. 48+sphingosine derivative So-61; No. 41+No. 38+No. 48+sphingosine derivative So-62; No. 41+No. 38+No. 48+sphingosine derivative So-63; No. 41+No. 38+No. 48+sphingosine derivative So-64; No. 41+No. 38+No. 48+sphingosine derivative So-67; No. 41+No. 38+No. 48+sphingosine derivative So-68; No. 41+No. 38+No. 48+sphingosine derivative So-69; No. 41+No. 38+No. 48+sphingosine derivative So-70; No. 41+No. 38+any one or more of sphingosine derivative So-23, 25, 26, 27, 29, 30, 31, 32, 33, 34, 35, 36, 37, 38, 40, 41, 42, 43, 44, 70, 71 or 73; wherein the compounds above are used at concentrations as shown in Table 1.

11. The method of claim 1, wherein the nucleic acid is synthetic or purified, therapeutic or non-Therapeutic, and/or diagnostic or non-diagnostic, single-stranded or double-stranded or partially double-stranded RNA or DNA;
when the nucleic acid is therapeutic or diagnostic, the nucleic acid is used to treat or diagnose a disease selected from the group consisting of: inflammatory diseases, pneumonia, myocarditis, acute and chronic gastritis, acute and chronic enteritis, acute and chronic hepatitis, acute and chronic nephritis, dermatitis, encephalitis, lymphitis, conjunctivitis, keratitis, iridocyclitis, otitis media, allergic rhinitis, asthma, pulmonary fibrosis, chronic obstructive pulmonary disease, allergic dermatitis, sickle cell disease, multiple sclerosis, systemic lupus erythematosus, lupus nephritis, lung cancer, gastric cancer, colorectal cancer, liver cancer, pancreatic cancer, cervical cancer, breast cancer, leukemia, multiple myeloma, diabetes, and gout.

12. The method of claim 11, wherein the RNA is selected from the group consisting of messenger RNA (mRNA), rRNA (ribosomal RNA), tRNA (transfer RNA), heterogeneous nuclear RNA (hnRNA), small nuclear RNA (snRNA), small nucleolar RNA (snoRNA), small cytoplasmic RNA, small RNA, transfer-messenger RNA (tmRNA), telomerase RNA and antisense RNA,

13. The method of claim 12, wherein the length of the small RNA is 14-32 bp, 16-28 bp, or 18-24 bp.

14. The method of claim 13, herein the small RNA comprises the sequence of
GTTCAGAGTTCTACAGTCCGA.

15. The method of claim 1, wherein the lipid composition is mixed with the nucleic acid by heating method, reverse evaporation method, direct mixing, repeated freeze-thaw and/or thin film dispersion, to obtain a lipid-nucleic acid mixture.

16. The method of claim 15, wherein:
the heating method comprises adding an organic solvent solution comprising lipid composition to an aqueous solution comprising nucleic acid, then heating at a temperature selected from 25° C. to 100° C., 50° C. to 100° C., 80° C. to 100° C., or 95° C. to 100° C. to obtain the lipid-nucleic acid mixture:
the reverse evaporation comprises adding an aqueous solution of nucleic acid to an organic solvent solution of the lipid composition, ultrasonicating, evaporating to remove the organic solvent, and then hydrating to obtain the lipid-nucleic acid mixture, wherein the reverse evaporation method is conducted at a temperature of 25° C. to 70° C. for a heating time of 0 minutes to 24 hours, 5 minutes to 20 hours, 5 minutes to 16 hours, 5 minutes to 10 hours, 5 minutes to 4 hours, or 10 minutes to 1 hour.

17. The method of claim 1, wherein the nucleic acid is delivered in vitro to the cell, or delivered in vivo to the subject.

18. The method of claim 17, wherein delivering in vivo to the subject comprises oral administration, intravenous administration, subcutaneous administration, intramuscular administration, intraarterial administration, intraperitoneal administration, intrapulmonary administration, intracerebral and intraspinal administration, intra-articular administration, intrasynovial administration, intrathecal administration, intra-trauma administration, and/or administration via inhalation paths.

19. A pharmaceutical composition, comprising a lipid composition and a nucleic acid, wherein the lipid composition comprises one or more compounds of Formula (I), a stereoisomer or a pharmaceutical acceptable salt thereof: 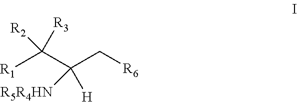
wherein:
R1 is selected from C₁₋₂₀alkyl or C₂₋₂₀alkenyl, which is optionally substituted by one to three hydroxyl groups;
R2 is hydrogen and R3 is hydroxyl; or
R2 and R3 together form oxo (═O);
R4 and R5 are independently selected from the group consisting of hydrogen, C₁₋₂₀alkyl, C₁₋₃₀alkylacyl and C₁₋₃₀alkenylacyl, said C₁₋₃₀alkylacyl and C₁₋₃₀alkenylacyl are optionally substituted by one to three groups selected from biotin acyl or hydroxyl;
or —NR4R5 group is a quaternary ammonium cation;
R6 is selected from the group consisting of hydrogen, hydroxyl, phosphate ester group, —O-glycosyl, Ganglioside, and aminoethoxyphosphonate ester group (NH₂—CH₂—CH₂—O—P(O)OH—).

20. A kit comprising a lipid composition,
the lipid composition comprising one or more compounds of Formula (I), a stereoisomer or a pharmaceutical acceptable salt thereof: 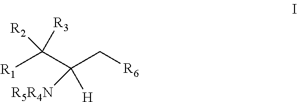
wherein:
R1 is selected from C₁₋₂₀alkyl or C₂₋₂₀alkenyl, which is optionally substituted by one to three hydroxyl groups;
R2 is hydrogen and R3 is hydroxyl; or
R2 and R3 together form oxo (═O);
R4 and R5 are independently selected from the group consisting of hydrogen C₁₋₂₀alkyl, C₁₋₃₀alkylacyl and C₁₋₃₀alkenylacyl, said C₁₋₃₀alkylacyl and C₁₋₃₀alkenylacyl are optionally substituted by one to three groups selected from biotin acyl or hydroxyl;
or —NR4R5 group is a quaternary ammonium cation;
R6 is selected from the group consisting of hydrogen, hydroxyl, phosphate ester group, —O-glycosyl, Ganglioside, and aminoethoxyphosphonate ester group (NH₂—CH₂—CH₂—O—P(O)OH—).


SEQUENCE LISTING (7 sequences)

SEQ ID NO: 1 - DNA, 21 bp, synthetic construct
  misc_feature 1..21: note = Synthetic
  source 1..21: mol_type = other DNA
gttcagagttctacagtccga

SEQ ID NO: 2 - DNA, 53 bp, synthetic construct
  misc_feature 1..53: note = Synthetic
  source 1..53: mol_type = other DNA
gtcgtatccagtgcagggtccgaggtattcgcactggatacgacaaaaatatg

SEQ ID NO: 3 - DNA, 50 bp, synthetic construct
  misc_feature 1..50: note = Synthetic
  source 1..50: mol_type = other DNA
gtcgtatccagtgcacgctccgaggtattcgcactggatacgactcggac

SEQ ID NO: 4 - DNA, 18 bp, synthetic construct
  misc_feature 1..18: note = Synthetic
  source 1..18: mol_type = other DNA
gcgcgtcgtgaagcgttc

SEQ ID NO: 5 - DNA, 16 bp, synthetic construct
  misc_feature 1..16: note = Synthetic
  source 1..16: mol_type = other DNA
gtgcagggtccgaggt

SEQ ID NO: 6 - DNA, 17 bp, synthetic construct
  misc_feature 1..17: note = Synthetic
  source 1..17: mol_type = other DNA
tcgcgcgttcagagttc

SEQ ID NO: 7 - DNA, 16 bp, synthetic construct
  misc_feature 1..16: note = Synthetic
  source 1..16: mol_type = other DNA
gtgcacgctccgaggt